(19) United States
(12) Patent Application Publication
(10) Pub. No.: US 2023/0355804 A1
(43) Pub. Date: Nov. 9, 2023

(54) VIRUSES ENGINEERED TO PROMOTE THANOTRANSMISSION AND THEIR USE IN TREATING CANCER

(71) Applicant: Flagship Pioneering Innovations V, Inc., Cambridge, MA (US)

(72) Inventors: Darby Rye Schmidt, Arlington, MA (US); Niranjana Aditi Nagarajan, Oakland, CA (US); William Joseph Kaiser, Tenafly, NJ (US); Peter Joseph Gough, Wayne, PA (US); Sabin Dhakal, Brookline, MA (US)

(21) Appl. No.: 18/013,409

(22) PCT Filed: Jun. 29, 2021
(86) PCT No.: PCT/US2021/039717

§ 371 Date: Dec. 28, 2022

Related U.S. Application Data
(60) Provisional application No. 63/169,166, filed on Mar. 31, 2021.
(60) Provisional application No. 63/045,610, filed on Jun. 29, 2020.

(51) Int. Cl. A61K 48/00; C12N 7/00; A61K 39/09; A61K 39/07; A61K 39/00; A61K 39/395; A61K 31/167; A61K 31/18; A61K 31/4045; A61K 31/165; A61K 31/4406; A61K 31/506; A61P 35/00; C07K 14/705; C07K 14/535; C07K 14/545; C07K 14/55; C07K 14/54; C07K 14/525; C07K 14/57; C12N 9/12

(52) U.S. Cl. CPC A61K 48/0041; C12N 7/00; A61K 39/092; A61K 39/07; A61K 39/4631; A61K 39/3955; A61K 31/167; A61K 31/18; A61K 31/4045; A61K 31/165; A61K 31/4406; A61K 31/506; A61P 35/00; C07K 14/70578; C07K 14/705; C07K 14/535; C07K 14/545; C07K 14/55; C07K 14/5406; C07K 14/5434; C07K 14/5443; C07K 14/525; C07K 14/57; C12N 9/12; C12Y 207/11001; C12N 2710/16622; C12N 2710/16671; C12N 2710/24122; C12N 2710/16632; A61K 2039/585

(57) ABSTRACT

In certain aspects, the disclosure relates to a virus engineered to comprise one or more polynucleotides that promote thanotransmission by a target cell. Thanotransmission is communication between cells that is a result of activation of a cell turnover pathway in a target cell, which signals a responding cell to undergo a biological response. Methods of promoting thanotransmission by a target cell, methods of promoting an immune response in a subject, and methods of treating cancer in a subject are also disclosed.

### RELATED APPLICATIONS

This application claims priority to U.S. Provisional Application No. 63/045,610, filed on Jun. 29, 2020, and U.S. Provisional Application No. 63/169,166, filed on Mar. 31, 2021, the entire contents of each of which are expressly incorporated herein by reference.

### BACKGROUND

In metazoans, programmed cell death is an essential genetically programmed process that maintains tissue homeostasis and eliminates potentially harmful cells.

### SUMMARY OF THE INVENTION

Thanotransmission is a process of communication between cells, e.g., between a target signaling cell and a responding cell, that is a result of activation of a cell turnover pathway in the target cell, which signals the responding cell to undergo a biological response. Thanotransmission may be induced in a target cell by modulation of cell turnover pathway genes through, for example, contacting the target cell with the engineered viruses described herein. The target cell in which a cell turnover pathway has been activated may signal a responding cell through factors actively released by the target cell, or through intracellular factors of the target cell that become exposed to the responding cell during the turnover (e.g., cell death) of the target cell.

In certain aspects, the disclosure relates to a virus engineered to comprise one or more polynucleotides that promote thanotransmission by a target cell. In one embodiment, at least one of the polynucleotides is heterologous to the virus. In one embodiment, at least one of the polynucleotides is heterologous to the target cell. In one embodiment, at least one of the polynucleotides promotes thanotransmission by the target cell by increasing expression or activity in the target cell of a thanotransmission polypeptide. In one embodiment, at least one of the polynucleotides encodes a thanotransmission polypeptide. In one embodiment, at least one of the polynucleotides promotes thanotransmission by the target cell by reducing expression or activity in the target cell of a polypeptide that suppresses thanotransmission. In one embodiment, at least one of the polynucleotides encodes an RNA molecule that reduces expression or activity in the target cell of a polypeptide that suppresses thanotransmission. In one embodiment, expression of at least one of the polynucleotides in the target cell alters a cell turnover pathway in the target cell. In one embodiment, at least one of the polynucleotides encodes a wild type protein.

In one embodiment, at least one of the polynucleotides encodes a death fold domain. In one embodiment, the death fold domain is selected from the group consisting of a death domain, a pyrin domain, a Death Effector Domain (DED), a C-terminal caspase recruitment domain (CARD), and variants thereof. In one embodiment, the death domain is from a protein selected from the group consisting of Fas-associated protein with death domain (FADD), Fas, Tumor necrosis factor receptor type 1 associated death domain (TRADD), Tumor necrosis factor receptor type 1 (TNFR1), and variants thereof. In one embodiment, the pyrin domain is from a protein selected from the group consisting of NLR Family Pyrin Domain Containing 3 (NLRP3) and apoptosis-associated speck-like protein (ASC). In one embodiment, the Death Effector Domain (DED) is from a protein selected from the group consisting of Fas-associated protein with death domain (FADD), caspase-8 and caspase-10.

In one embodiment, the CARD is from a protein selected from the group consisting of RIP-associated ICH1/CED3-homologous protein (RAIDD), apoptosis-associated speck-like protein (ASC), mitochondrial antiviral-signaling protein (MAVS), caspase-1, and variants thereof. In one embodiment, at least one of the polynucleotides encodes a Toll/interleukin-1 receptor (TIR) domain. In one embodiment, the TIR domain is from a protein selected from the group consisting of Myeloid Differentiation Primary Response Protein 88 (MyD88), Toll/interleukin-1 receptor (TIR)-domain-containing adapter-inducing interferon-β (TRIF), Toll Like Receptor 3 (TLR3), Toll Like Receptor 4 (TLR4), TIR Domain Containing Adaptor Protein (TIRAP), and Translocating chain-associated membrane protein (TRAM)

In one embodiment, at least one of the polynucleotides encodes a protein comprising a TIR domain. In one embodiment, the protein comprising a TIR domain is selected from the group consisting of Myeloid Differentiation Primary Response Protein 88 (MyD88), Toll/interleukin-1 receptor (TIR)-domain-containing adapter-inducing interferon-β (TRIF), Toll Like Receptor 3 (TLR3), Toll Like Receptor 4 (TLR4), TIR Domain Containing Adaptor Protein (TIRAP) and Translocating chain-associated membrane protein (TRAM).

In one embodiment, the one or more polynucleotides encode any one or more of receptor-interacting serine/threonine-protein kinase 3 (RIPK3), Z-DNA-binding protein 1 (ZBP1), mixed lineage kinase domain like pseudokinase (MLKL), Toll/interleukin-1 receptor (TIR)-domain-containing adapter-inducing interferon-β (TRIF), an N-terminal truncation of TRIF that comprises only a TIR domain and a RHIM domain, Interferon Regulatory Factor 3 (IRF3), Fas-associated protein with death domain (FADD), a truncated FADD, Tumor necrosis factor receptor type 1 associated death domain (TRADD), and Cellular FLICE (FADD-like IL-1(3-converting enzyme)-inhibitory protein (c-FLIP).

In one embodiment, the polynucleotide encoding ZBP1 comprises a deletion of receptor-interacting protein homotypic interaction motif (RHIM) C, a deletion of RHIM D, and a deletion at the N-terminus of a Zα1 domain. In one embodiment, at least one of the polynucleotides inhibits expression or activity of receptor-interacting serine/threonine-protein kinase 1 (RIPK1).

In one embodiment, at least one of the polynucleotides encodes a fusogenic protein. In one embodiment, the fusogenic protein is glycoprotein from gibbon ape leukemia virus (GALV) and has the R transmembrane peptide mutated or removed (GALV-R-). In one embodiment, at least one of the polynucleotides encodes an immune stimulatory protein. In one embodiment, the immune stimulatory protein is an antagonist of transforming growth factor beta (TGF-β), a colony-stimulating factor, a cytokine, or an immune checkpoint modulator. In one embodiment, the colony-stimulating factor is granulocyte-macrophage colony-stimulating factor (GM-CSF). In one embodiment, the polynucleotide encoding GM-CSF is inserted into the ICP34.5 gene locus of the virus. In one embodiment, the cytokine is an interleukin. In one embodiment, the interleukin is selected from the group consisting of IL-1α, IL-1β, IL-2, IL-4, IL-12, IL-15, IL-18, IL-21, IL-24, IL-33, IL-36a, IL-36β and IL-36γ. In one embodiment, the cytokine is selected from the group consisting of a type I interferon, interferon gamma, a type III interferon and TNF alpha.

In one embodiment, the immune checkpoint modulator is an antagonist of an inhibitory immune checkpoint protein. In one embodiment, the inhibitory immune checkpoint protein is selected from the group consisting of ADORA2A, B7-H3, B7-H4, IDO, KIR, VISTA, PD-1, PD-L1, PD-L2, LAG3, Tim3, BTLA and CTLA4. In one embodiment, the immune checkpoint modulator is an agonist of a stimulatory immune checkpoint protein. In one embodiment, the stimulatory immune checkpoint protein is selected from the group consisting of CD27, CD28, CD40, CD122, OX40, GITR, ICOS and 4-1BB. In one embodiment, the agonist of the stimulatory immune checkpoint protein is selected from CD40 ligand (CD40L), ICOS ligand, GITR ligand, 4-1-BB ligand, 0X40 Ligand and a modified version of any thereof. In one embodiment, the agonist of the stimulatory immune checkpoint protein is an antibody agonist of a protein selected from CD40, ICOS, GITR, 4-1-BB and 0X40. In one embodiment, the immune stimulatory protein is an flt3 ligand or an antibody agonist of flt3.

In one embodiment, at least one of the polynucleotides is a suicide gene. In one embodiment, the suicide gene encodes a polypeptide selected from the group consisting of FK506 binding protein (FKBP)-FAS, FKBP-caspase-8, FKBP-caspase-9, a polypeptide having cytosine deaminase (CDase) activity, a polypeptide having thymidine kinase activity, a polypeptide having uracil phosphoribosyl transferase (UPRTase) activity, and a polypeptide having purine nucleoside phosphorylase activity. In one embodiment, the polypeptide having CDase activity is FCY1, FCA1 or CodA. In one embodiment, the polypeptide having UPRTase activity is FUR1 or a variant thereof. In one embodiment, the variant of FUR1 is FUR1Δ105. In one embodiment, the suicide gene encodes a chimeric protein having CDase and UPRTase activity. In one embodiment, the chimeric protein is selected from the group consisting of codA::upp, FCY1::FUR1, FCY1::FUR1Δ105 (FCU1) and FCU1-8 polypeptides.

In one embodiment, at least one of the polynucleotides encodes a polypeptide selected from the group consisting of gasdermin-A (GSDM-A), gasdermin-B (GSDM-B), gasdermin-C (GSDM-C), gasdermin-D (GSDM-D), gasdermin-E (GSDM-E), apoptosis-associated speck-like protein containing C-terminal caspase recruitment domain (ASC-CARD) with a dimerization domain, and mutants thereof.

In some embodiments, the one or more polynucleotides that promote thanotransmision encode two or more different thanotransmission polypeptides, wherein the two or more thanotransmission polypeptides are selected from the group consisting of TRADD, TRAF2, TRAF6, cIAP1, cIAP2, XIAP, NOD2, MyD88, TRAM, HOIL, HOIP, Sharpin, IKKg, IKKa, IKKb, RelA, MAVS, RIGI, MDA5, Tak1, TBK1, IKKe, IRF3, IRF7, IRF1, TRAF3, a Caspase, FADD, TNFR1, TRAILR1, TRAILR2, FAS, Bax, Bak, Bim, Bid, Noxa, Puma, TRIF, ZBP1, RIPK1, RIPK3, MLKL, Gasdermin A, Gasdermin B, Gasdermin C, Gasdermin D, Gasdermin E, a tumor necrosis factor receptor superfamily (TNFSF) protein, variants thereof, and functional fragments thereof. In some embodiments, at least one of the polynucleotides encodes a chimeric protein comprising at least two of the thanotransmission polypeptides. In some embodiments, at least one of the polynucleotides is transcribed as a single transcript that encodes the two or more different thanotransmission polypeptides.

In some embodiments, at least two of the thanotransmission polypeptides encoded by the one or more polynucleotides activate NF-kB. In some embodiments, at least two of the thanotransmission polypeptides encoded by the one or more polynucleotides activate IRF3 and/or IRF7. In some embodiments, at least two of the thanotransmission polypeptides encoded by the one or more polynucleotides promote extrinsic apoptosis. In some embodiments, at least two of the thanotransmission polypeptides encoded by the one or more polynucleotides promote programmed necrosis. In some embodiments, at least one of the thanotransmission polypeptides encoded by the one or more thanotransmission polynucleotides activates NF-kB, and at least one of the thanotransmission polypeptides encoded by the one or more polynucleotides activates IRF3 and/or IRF7. In some embodiments, at least one of the thanotransmission polypeptides encoded by the one or more polynucleotides activates NF-kB, and at least one of the thanotransmission polypeptides encoded by the one or more polynucleotides promotes extrinsic apoptosis. In some embodiments, at least one of the thanotransmission polypeptides encoded by the one or more polynucleotides activates NF-kB, and at least one of the thanotransmission polypeptides encoded by the one or more polynucleotides promotes programmed necrosis. In some embodiments, at least one of the thanotransmission polypeptides encoded by the one or more polynucleotides activates IRF3 and/or IRF7, and at least one of the thanotransmission polypeptides encoded by the one or more polynucleotides promotes extrinsic apoptosis. In some embodiments, at least one of the thanotransmission polypeptides encoded by the one or more thanotransmission polynucleotides activates IRF3 and/or IRF7, and at least one of the thanotransmission polypeptides encoded by the one or more polynucleotides promotes programmed necrosis. In some embodiments, at least one of the thanotransmission polypeptides encoded by the one or more polynucleotides promotes extrinsic apoptosis, and at least one of the thanotransmission polypeptides encoded by the one or more thanotransmission polynucleotides promotes programmed necrosis. In some embodiments, the programmed necrosis comprises necroptosis. In some embodiments, the programmed necrosis comprises pyroptosis.

In some embodiments, the thanotransmission polypeptide that activates NF-kB is selected from the group consisting of TRIF, TRADD, TRAF2, TRAF6, cIAP1, cIAP2, XIAP, NOD2, MyD88, TRAM, HOIL, HOIP, Sharpin, IKKg, IKKa, IKKb, RelA, MAVS, RIGI, MDA5, Tak1, a TNFSF protein, and functional fragments thereof. In some embodiments, the thanotransmission polypeptide that activates IRF3 and/or IRF7 is selected from the group consisting of TRIF, MyD88, MAVS, TBK1, IKKe, IRF3, IRF7, IRF1, TRAF3 and functional fragments thereof. In some embodiments, the thanotransmission polypeptide that promotes extrinsic apoptosis is selected from the group consisting of TRIF, RIPK1, Caspase, FADD, TRADD, TNFR1, TRAILR1, TRAILR2, FAS, Bax, Bak, Bim, Bid, Noxa, Puma, and functional fragments thereof. In some embodiments, the thanotransmission polypeptide that promotes programmed necrosis is selected from the group consisting of TRIF, ZBP1, RIPK1, RIPK3, MLKL, a Gasdermin, and functional fragments thereof.

In some embodiments, at least one of the thanotransmission polypeptides comprises TRIF or a functional fragment thereof. In some embodiments, at least one of the thanotransmission polypeptides comprises RIPK3 or a functional fragment thereof. In some embodiments, at least one of the thanotransmission polypeptides encoded by the one or more thanotransmission polynucleotides comprises TRIF or a functional fragment thereof, and at least one of the thanotransmission polypeptides encoded by the one or more polynucleotides comprises RIPK3 or a functional fragment thereof. In some embodiments, at least one of the thanotransmission polypeptides comprises MAVS or a functional fragment thereof, and at least one of the thanotransmission polypeptides comprises RIPK3 or a functional fragment thereof.

In some embodiments, the one or more polynucleotides further encode a polypeptide that inhibits caspase activity. In some embodiments, the polypeptide that inhibits caspase activity is selected from the group consisting of a FADD dominant negative mutant (FADD-DN), cFLIP, vICA, a caspase 8 dominant negative mutant (Casp8-DN), cIAP1, cIAP2, Tak1, an IKK, and functional fragments thereof. In some embodiments, the polypeptide that inhibits caspase activity is FADD-DN. In some embodiments, the polypeptide that inhibits caspase activity is cFLIP. In some embodiments, the polypeptide that inhibits caspase activity is vICA.

In some embodiments, the virus encodes at least one Gasdermin or a functional fragment thereof. In some embodiments, at least one of the thanotransmission polypeptides comprises TRIF or a functional fragment thereof, and at least one of the thanotransmission polypeptides comprises RIPK3 or a functional fragment thereof, and at least one of the thanotransmission polypeptides comprises a Gasdermin or a functional fragment thereof. In some embodiments, at least one of the thanotransmission polypeptides comprises MAVS or a functional fragment thereof, and at least one of the thanotransmission polypeptides comprises RIPK3 or a functional fragment thereof, and at least one of the thanotransmission polypeptides comprises a Gasdermin or a functional fragment thereof. In some embodiments, the Gasdermin is Gasdermin E or a functional fragment thereof.

In some embodiments, the virus further comprises at least one polynucleotide encoding a dimerization domain. In some embodiments, at least one of the thanotransmission polypeptides is comprised within a fusion protein that further comprises a dimerization domain. In some embodiments, the dimerization domain is heterologous to the thanotransmission polypeptide.

In certain aspects, the disclosure relates to a pharmaceutical composition comprising one or more of the viruses disclosed herein, and a pharmaceutically acceptable carrier. In certain aspects, the disclosure relates to a method of delivering one or more thanotransmission polynucleotides to a subject, the method comprising administering the pharmaceutical composition to the subject. In certain aspects, the disclosure relates to a method of promoting thanotransmission in a subject, the method comprising administering the pharmaceutical composition to the subject in an amount and for a time sufficient to promote thanotransmission. In certain aspects, the disclosure relates to a method of increasing immune response in a subject in need thereof, the method comprising administering the pharmaceutical composition to the subject in an amount and for a time sufficient to increase immune response in the subject. In certain aspects, the disclosure relates to a method of treating a cancer in a subject in need thereof, the method comprising administering the pharmaceutical composition to the subject in an amount and for a time sufficient to treat the cancer.

In one embodiment, administering the pharmaceutical composition to the subject reduces proliferation of cancer cells in the subject. In one embodiment, the proliferation of the cancer cells is a hyperproliferation of the cancer cells resulting from a cancer therapy administered to the subject. In one embodiment, administering the pharmaceutical composition to the subject reduces metastasis of cancer cells in the subject. In one embodiment, administering the pharmaceutical composition to the subject reduces neovascularization of a tumor in the subject. In one embodiment, treating a cancer comprises any one or more of reduction in tumor burden, reduction in tumor size, inhibition of tumor growth, achievement of stable cancer in a subject with a progressive cancer prior to treatment, increased time to progression of the cancer, and increased time of survival.

In one embodiment, the pharmaceutical composition is administered intravenously to the subject. In one embodiment, the pharmaceutical composition is administered intratumorally to the subject. In one embodiment, the subject was previously treated with an immunotherapy. In one embodiment, the cancer is not responsive to an immunotherapy. In one embodiment, the cancer is a cancer responsive to an immunotherapy. In one embodiment, administration of the pharmaceutical composition to the subject improves response of the cancer to an immunotherapy relative to a subject that is administered the immunotherapy but is not administered the virus. In one embodiment, the immunotherapy is an immune checkpoint therapy. In one embodiment, the immune checkpoint therapy is an immune checkpoint inhibitor therapy.

In one embodiment, the cancer is selected from a carcinoma, sarcoma, lymphoma, melanoma, and leukemia. In one embodiment, the cancer is a solid tumor. In one embodiment, the cancer is selected from the group consisting of melanoma, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplasia syndrome, multiple myeloma, transitional cell carcinoma, neuroblastoma, plasma cell neoplasms, Wilm's tumor, and hepatocellular carcinoma. In one embodiment, the cancer is colon cancer.

In one embodiment, the method further comprises administering an anti-neoplastic agent to the subject. In one embodiment, the anti-neoplastic agent is a chemotherapeutic agent. In one embodiment, the anti-neoplastic agent is a biologic agent. In one embodiment, the biologic agent is an antigen binding protein. In one embodiment, the anti-neoplastic agent is an immunotherapeutic. In one embodiment, the immunotherapeutic is selected from the group consisting of a Toll-like receptor (TLR) agonist, a cell-based therapy, a cytokine, a cancer vaccine, and an immune checkpoint modulator of an immune checkpoint molecule. In one embodiment, the TLR agonist is selected from Coley's toxin and Bacille Calmette-Guérin (BCG). In one embodiment, the cell-based therapy is a chimeric antigen receptor T cell (CAR-T cell) therapy. In one embodiment, the immune checkpoint molecule is selected from CD27, CD28, CD40, CD122, OX40, GITR, ICOS, 4-1BB, ADORA2A, B7-H3, B7-H4, BTLA, CTLA-4, IDO, KIR, LAG-3, PD-1, PD-L1, PD-L2, TIM-3, and VISTA. In one embodiment, the immune checkpoint molecule is a stimulatory immune checkpoint molecule and the immune checkpoint modulator is an agonist of the stimulatory immune checkpoint molecule. In one embodiment, the immune checkpoint molecule is an inhibitory immune checkpoint molecule and the immune checkpoint modulator is an antagonist of the inhibitory immune checkpoint molecule. In one embodiment, the immune checkpoint modulator is selected from a small molecule, an inhibitory RNA, an antisense molecule, and an immune checkpoint molecule binding protein. In one embodiment, the immune checkpoint molecule is PD-1 and the immune checkpoint modulator is a PD-1 inhibitor. In one embodiment, the PD-1 inhibitor is selected from pembrolizumab, nivolumab, pidilizumab, SHR-1210, MEDI0680R01, BBg-A317, TSR-042, REGN2810 and PF-06801591. In one embodiment, the immune checkpoint molecule is PD-L1 and the immune checkpoint modulator is a PD-L1 inhibitor. In one embodiment, the PD-L1 inhibitor is selected from durvalumab, atezolizumab, avelumab, MDX-1105, AMP-224 and LY3300054. In one embodiment, the immune checkpoint molecule is CTLA-4 and the immune checkpoint modulator is a CTLA-4 inhibitor. In one embodiment, the CTLA-4 inhibitor is selected from ipilimumab, tremelimumab, JMW-3B3 and AGEN1884.

In one embodiment, the anti-neoplastic agent is a histone deacetylase inhibitor. In one embodiment, the histone deacetylase inhibitor is a hydroxamic acid, a benzamide, a cyclic tetrapeptide, a depsipeptide, an electrophilic ketone, or an aliphatic compound. In one embodiment, the hydroxamic acid is vorinostat (SAHA), belinostat (PXD101), LAQ824, trichostatin A, or panobin ostat (LBH589). In one embodiment, the benzamide is entinostat (MS-275), 01994, or mocetinostat (MGCD0103). In one embodiment, the cyclic tetrapeptide is trapoxin B. In one embodiment, the aliphatic acid is phenyl butyrate or valproic acid.

In some embodiments, the virus is not an adenovirus or an adeno-associated virus (AAV). In some embodiments, the virus the virus is cytolytic. In some embodiments, the virus preferentially infects dividing cells. In some embodiments, the virus is capable of reinfecting a host that was previously infected. In some embodiments, the virus does not comprise a polynucleotide encoding a synthetic multimerization domain. In some embodiments, the virus the virus is not a Vaccinia virus. In some embodiments, the virus does not comprise a polynucleotide encoding TRIF.

In one embodiment, an immuno-stimulatory cell turnover pathway is induced in the target cell. In one embodiment, the immuno-stimulatory cell turnover pathway is selected from the group consisting of programmed necrosis (e.g., necroptosis or pyroptosis), extrinsic apoptosis, ferroptosis and combinations thereof. In one embodiment, the target cell is deficient in the immuno-stimulatory cell turnover pathway. In one embodiment, the target cell has an inactivating mutation in one or more of a gene encoding receptor-interacting serine/threonine-protein kinase 3 (RIPK1), a gene encoding receptor-interacting serine/threonine-protein kinase 3 (RIPK3), a gene encoding Z-DNA-binding protein 1 (ZBP1), a gene encoding mixed lineage kinase domain like pseudokinase (MLKL), and a gene encoding Toll/interleukin-1 receptor (TIR)-domain-containing adapter-inducing interferon-β (TRIF). In one embodiment, the target cell has reduced expression or activity of one or more of RIPK1, RIPK3, ZBP1, TRIF, and MLKL. In one embodiment, the target cell has copy number loss of one or more of a gene encoding RIPK1, a gene encoding RIPK3, a gene encoding ZBP1, a gene encoding TRIF, and a gene encoding MLKL. In one embodiment, the target cell is selected from the group consisting of a cancer cell, an immune cell, an endothelial cell and a fibroblast. In one embodiment, the target cell is a cancer cell. In one embodiment, the cancer is a metastatic cancer.

In one embodiment, the virus is an oncolytic virus. In one embodiment, the virus is a DNA replicative virus. In one embodiment, the virus is a DNA replicative oncolytic virus. In one embodiment, the virus preferentially infects the target cell. In one embodiment, the virus comprises inactivating mutations in one or more endogenous viral genes that inhibit thanotransmission by the cancer cell. In one embodiment, the virus is capable of transporting a heterologous polynucleotide of at least 4 kb into a target cell.

In one embodiment, the virus is herpes simplex virus (HSV). In one embodiment, the HSV is HSV1. In one embodiment, the HSV1 is selected from the group consisting of Kos, F1, MacIntyre, McKrae and related strains. In one embodiment, the HSV is defective in one or more genes selected from the group consisting of ICP34.5, ICP47, UL24, UL55, UL56. In one embodiment, each ICP34.5 encoding gene is replaced by a polynucleotide cassette comprising a US 11 encoding gene operably linked to an immediate early (IE) promoter. In one embodiment, the HSV comprises a ΔZα mutant form of a Vaccinia virus E3L gene. In one embodiment, the HSV is defective in one or more functions of ICP6. In one embodiment, the ICP6 has a mutation of the receptor-interacting protein homotypic interaction motif (RHIM) domain. In one embodiment, the ICP6 has one or more mutations at the C-terminus that inhibit caspase-8 binding. In one embodiment, the HSV expresses the US11 gene as an immediate early gene. In one embodiment, the ICP47 gene is deleted such that the US11 gene is under the control of an ICP47 immediate early promoter.

In one embodiment, the virus belongs to the Poxviridae family. In one embodiment, the virus that belongs to the Poxviridae family is selected from the group consisting of myxoma virus, Yaba-like disease virus, raccoonpox virus, orf virus and cowpox virus. In one embodiment, the virus belongs to the Chordopoxvirinae subfamily of the Poxviridae family. In one embodiment, the virus belongs to the Orthopoxvirus genus of the Chordopoxvirinae subfamily. In one embodiment, the virus belongs to the Vaccinia virus species of the Orthopoxvirus genus. In one embodiment, the Vaccinia virus is a strain selected from the group consisting of Dairenl, IHD-J, L-IPV, LC16M8, LC16MO, Lister, LIVP, Tashkent, WR 65-16, Wyeth, Ankara, Copenhagen, Tian Tan and WR. In one embodiment, the Vaccinia virus is engineered to lack thymidine kinase (TK) activity. In one embodiment, the Vaccinia virus has an inactivating mutation or deletion in the J2R gene that reduces or eliminates TK activity. In one embodiment, the Vaccinia virus is engineered to lack ribonucleotide reductase (RR) activity. In one embodiment, the Vaccinia virus has an inactivating mutation or deletion in a gene selected from I4L and F4L gene that reduces or eliminates RR activity. In one embodiment, the Vaccinia virus is defective in the E3L gene. In one embodiment, the E3L gene has a mutation that results in induction of necroptosis in the cancer cell. In one embodiment, the virus is an adenovirus. In one embodiment, the adenovirus is Ad5/F35. In one embodiment, the adnovirus comprises a deletion in the Adenovirus Early Region 1A (E1A). In one embodiment, the adenovirus comprises a deletion in the Adenovirus Early Region 1B (E1B). In one embodiment, the adenovirus has an Arg-Gly-Asp (RGD)-motif engineered into a fiber-H loop.

### BRIEF DESCRIPTION OF THE FIGURES

FIGS. 1A shows a schematic of recombinant HSV1. FIG. 1B shows an exemplary thanotransmission cassette (TC) comprising genes encoding RIPK3, ZBP1, MLKL and TRIF.

FIG. 2 shows a schematic of recombinant HSV1 comprising insertion of a gene encoding an siRNA or gRNA/Cas9 into the ICP34.5 gene of HSV1.

FIG. 3 shows a schematic of recombinant HSV1 comprising insertion of a thanotransmission cassette (TC) into the ICP34.5 gene of HSV1 and insertion of a gene encoding a mutated RHIM domain into the ICP6 gene of HSV1.

FIG. 4A shows relative viability of CT-26 mouse colon carcinoma cells following induction of thanotransmission. FIG. 4B shows relative viability of CT-26 mouse colon carcinoma cells expressing TRIF alone or in combination with RIPK3 and or Gasdermin E.

FIG. 5A shows the effects of cell turnover factors (CTFs) generated from CT-26 mouse colon carcinoma cells following induction of thanotransmission polypeptide expression on stimulation of IFN-related gene activation in macrophages.. FIG. 5B shows the effects of cell turnover factors (CTFs) generated from CT-26 mouse colon carcinoma cells following induction of TRIF alone or in combination with RIPK3 (cR3) and/or Gasdermin E (cGE)) on stimulation of IFN-related gene activation in macrophages. In FIG. 5A, the Tet-inducible RIPK3 is designated as “RIPK3”, and the RIPK3 construct containing a constitutive PGK promoter is designated as “PGK_RIPK3”.

FIG. 6 shows the effects of cell turnover factors (CTFs) generated from CT-26 mouse colon carcinoma cells following induction of TRIF, RIPK3 or TRIF and RIPK3 expression on stimulation of expression of activation markers in bone marrow derived dendritic cells (BMDCs). MFI is mean-fluorescent intensity.

FIGS. 7A, 7B and 7C show the effects of thanotransmission polypeptide expression on survival of mice implanted with CT-26 mouse colon carcinoma cells. FIG. 7B shows percent survival of mice implanted with CT-26 mouse colon carcinoma cells and treated with an anti-PD1 antibody. “CT26-TF” represents CT-26 cells expressing TRIF alone, and “CT26-P_R3” represents cells expressing RIPK3 alone.

FIG. 8A shows relative NF-kB activity in THP-1 Dual cells treated with cell culture from U937 leukemia cells expressing various thanotransmission payloads and treated with caspase inhibitor (Q-VD-Oph) alone or in combination with RIPK3 inhibitor (GSK872). FIGS. 8B and 8C show relative IRF activity in THP-1 Dual cells treated with cell culture from U937 leukemia cells expressing various thanotransmission payloads and treated with caspase inhibitor (Q-VD-Oph) alone or in combination with RIPK3 inhibitor (GSK872). The U937 cells were also treated with doxycycline to induce thanotransmission polypeptide expression, alone or in combination with B/B homodimerizer to induce dimerization. In FIGS. 8A-8C, + indicates U937 cells treated with doxycycline, and ++ indicates U937 cells treated with doxycycline and B/B homodimerizer.

FIG. 9A shows relative viability of CT-26 mouse colon carcinoma cells expressing thanotransmission polypeptides alone or in combination with caspase inhibitors. FIG. 9B shows the effects of cell turnover factors (CTFs) generated from CT-26 mouse colon carcinoma cells following induction of thanotransmission polypeptide expression alone or in combination with caspase inhibitors on stimulation of IFN-related gene activation in macrophages. FIG. 9C shows the effect of TRIF+RIPK3 expression alone or in combination with caspase inhibitors on survival of mice implanted with CT-26 mouse colon carcinoma cells.

### DETAILED DESCRIPTION

The present disclosure relates to a virus engineered to comprise one or more polynucleotides that promote thanotransmission by a target cell. Thanotransmission is a process of communication between cells, e.g., between a target signaling cell and a responding cell, that is a result of activation of a cell turnover pathway in the target cell, which signals the responding cell to undergo a biological response. Thanotransmission may be induced in a target cell by modulation of cell turnover pathway genes through, for example, contacting the target cell with the engineered viruses described herein. The target cell in which a cell turnover pathway has been activated may signal a responding cell through factors actively released by the target cell, or through intracellular factors of the target cell that become exposed to the responding cell during the turnover (e.g., cell death) of the target cell. In various embodiments of the present disclosure, one or more polynucleotides comprised by the virus promote thanotransmission by the target cell by increasing expression or activity of one or more polypeptides that promote thanotransmission, and/or by reducing expression or activity of one or more polypeptides that suppress thanotransmission in the target cell.

In some embodiments, the virus is engineered to comprise a polynucleotide encoding only one polypeptide that promotes thanotransmission. In other embodiments, the virus is engineered to comprise one or more polynucleotides encoding two or more different polypeptides that promote thanotransmission. In some embodiments, the polypeptide(s) that promote thanotransmission (e.g., the only one polypeptide or the two or more different polypeptides) are selected from the group consisting of TRADD, TRAF2, TRAF6, cIAP1, cIAP2, XIAP, NOD2, MyD88, TRAM, HOIL, HOIP, Sharpin, IKKg, IKKa, IKKb, RelA, MAVS, RIGI, MDA5, Tak1, TBK1, IKKe, IRF3, IRF7, IRF1, TRAF3, a Caspase, FADD, TRADD, TNFR1, TRAILR1, TRAILR2, FAS, Bax, Bak, Bim, Bid, Noxa, Puma, TRIF, ZBP1, RIPK1, RIPK3, MLKL, Gasdermin A, Gasdermin B, Gasdermin C, Gasdermin D, Gasdermin E, a tumor necrosis factor receptor superfamily (TNFSF) protein, variants thereof, and functional fragments thereof.

Applicant has surprisingly discovered that modulation of thanotransmission can modulate (e.g., reduce activity, growth or viability of) a cancer cell. For example, expression of one or more polypeptides that promote thanotransmission (e.g., TRIF and RIPK3, either alone or in combination) in a cancer cell reduces viability of the cancer cell in vitro. In addition, Applicant has surprisingly shown that subjects harboring cancer cells engineered to express one or more polypeptides that promote thanotransmission (e.g. TRIF alone, or TRIF in combination with RIPK3) exhibit increased survival rates compared to subjects harboring cancer cells that have not been engineered to express a polypeptide that promotes thanotransmission. In particular, the combined expression of two polypeptides that promote thanotransmission (TRIF and RIPK3) was found to be more effective in increasing survival than either polypeptide alone. Combination of TRIF+RIPK3 with a caspase inhibitor (e.g, FADD-DN or vICA) or Gasdermin E was demonstrated to further increase survival. These results suggest that cancer cell growth may be reduced in a subject through administration of a virus engineered to comprise one or more polynucleotides that promote thanotransmission. For example, the engineered virus may transduce a cancer cell, resulting in expression of one or more polypeptide that promote thanotransmission, thereby reducing viability of the cancer cell and/or promoting host immune response against the cancer cell though the release of immune-stimulatory cell turnover factors.

Accordingly, the present disclosure also relates to methods of promoting thanotransmission by a target cell (e.g. a cancer cell) comprising contacting a target cell with a virus engineered to comprise one or more polynucleotides that promote thanotransmission by the target cell, wherein the target cell is contacted with the virus in an amount and for a time sufficient to promote thanotransmission by the target cell. Pharmaceutical compositions comprising the engineered viruses are also disclosed. The present disclosure further relates to methods of promoting thanotransmission in a subject, e.g., a subject diagnosed with cancer, the methods comprising administering the pharmaceutical composition to the subject in an amount and for a time sufficient to promote thanotransmission. Methods of increasing immune response in a subject in need thereof, and methods of treating a cancer in a subject in need thereof, are also disclosed.

### I. Definitions

The terms “administer”, “administering” or “administration” include any method of delivery of a pharmaceutical composition or agent into a subject's system or to a particular region in or on a subject.

As used herein, “administering in combination”, “co-administration” or “combination therapy” is understood as administration of two or more active agents using separate formulations or a single pharmaceutical formulation, or consecutive administration in any order such that, there is a time period while both (or all) active agents overlap in exerting their biological activities. It is contemplated herein that one active agent (e.g., a virus engineered to comprise one or more polynucleotides that promote thanotransmission by a target cell) can improve the activity of a second therapeutic agent (e.g. an immunotherapeutic), for example, can sensitize target cells, e.g., cancer cells, to the activities of the second therapeutic agent or can have a synergistic effect with the second therapeutic agent. “Administering in combination” does not require that the agents are administered at the same time, at the same frequency, or by the same route of administration. As used herein, “administering in combination”, “co-administration” or “combination therapy” includes administration of a virus engineered to comprise one or more polynucleotides that promote thanotransmission by a target cell with one or more additional therapeutic agents, e.g., an immunotherapeutic (e.g. an immune checkpoint modulator). Examples of immunotherapeutics are provided herein.

As used herein, the terms “increasing” and “decreasing” refer to modulating resulting in, respectively, greater or lesser amounts, function or activity of a parameter relative to a reference. For example, subsequent to administration of a composition described herein, a parameter (e.g., activation of IRF, activation of NFkB, activation of macrophages, size or growth of a tumor) may be increased or decreased in a subject by at least 5%, 10%, 15%, 20%, 25%, 30%, 35%, 40%, 45%, 50%, 55%, 60%, 65%, 70%, 75%, 80%, 85%, 90%, 95% or 98% or more relative to the amount of the parameter prior to administration. Generally, the metric is measured subsequent to administration at a time that the administration has had the recited effect, e.g., at least one day, one week, one month, 3 months, 6 months, after a treatment regimen has begun. Similarly, pre-clinical parameters (such as activation of NFkB or IRF of cells in vitro, and/or reduction in tumor burden of a test mammal, by a composition described herein) may be increased or decreased by at least 5%, 10%, 15%, 20%, 25%, 30%, 35%, 40%, 45%, 50%, 55%, 60%, 65%, 70%, 75%, 80%, 85%, 90%, 95% or 98% or more relative to the amount of the parameter prior to administration.

As used herein, “an anti-neoplastic agent” refers to a drug used for the treatment of cancer. Anti-neoplastic agents include chemotherapeutic agents (e.g., alkylating agents, antimetabolites, anti-tumor antibiotics, topoisomerase inhibitors, mitotic inhibitors corticosteroids, and enzymes), biologic anti-cancer agents, and immune checkpoint modulators.

A “cancer treatment regimen” or “anti-neoplastic regimen” is a clinically accepted dosing protocol for the treatment of cancer that includes administration of one or more anti-neoplastic agents to a subject in specific amounts on a specific schedule.

The term “functional fragment” as used herein with reference to a polypeptide refers to a portion of a polypeptide that retains at least one biological activity of the polypeptide, e.g. the ability to promote thanotransmission. In some embodiments, the functional fragment is a domain of the polypeptide, e.g. a death fold domain, a death domain, a pyrin domain, a Death Effector Domain (DED), or a C-terminal caspase recruitment domain (CARD) of the polypeptide. In some embodiments, a functional fragment of a polypeptide is a portion of a domain that retains at least one biological activity of the domain.

The terms “fusion protein” and “chimeric protein” are used herein interchangeably to refer to a protein comprising at least two polypeptides that do not occur within the same protein in nature.

A “fusogenic protein” as used herein refers to any heterologous protein capable of promoting fusion of a cell infected with a virus to another cell. Examples of fusogenic proteins include VSV-G, syncitin-1 (from human endogenous retrovirus-W (HERV-W)) or syncitin-2 (from HERVFRDE1), paramyxovirus SV5-F, measles virus-H, measles virus-F, RSV-F, the glycoprotein from a retrovirus or lentivirus, such as gibbon ape leukemia virus (GALV), murine leukemia virus (MLV), Mason-Pfizer monkey virus (MPMV) and equine infectious anemia virus (EIAV) with the R transmembrane peptide removed (R-versions).

The term “heterologous” as used herein refers to a combination of elements that do not naturally occur in combination. For example, a polynucleotide that is heterologous to a virus or target cell refers to a polynucleotide that does not naturally occur in the virus or target cell, or that occurs in a position in the virus or target cell that is different from the position at which it occurs in nature. A polypeptide that is heterologous to a target cell refers to a polypeptide that does not naturally occur in the target cell, or that is expressed from a polynucleotide that is heterologous to the target cell.

As used herein, an “immune checkpoint” or “immune checkpoint molecule” is a molecule in the immune system that modulates a signal. An immune checkpoint molecule can be a stimulatory checkpoint molecule, i.e., increase a signal, or inhibitory checkpoint molecule, i.e., decrease a signal. A “stimulatory checkpoint molecule” as used herein is a molecule in the immune system that increases a signal or is co-stimulatory. An “inhibitory checkpoint molecule”, as used herein is a molecule in the immune system that decreases a signal or is co-inhibitory.

As used herein, an “immune checkpoint modulator” is an agent capable of altering the activity of an immune checkpoint in a subject. In certain embodiments, an immune checkpoint modulator alters the function of one or more immune checkpoint molecules including, but not limited to, CD27, CD28, CD40, CD122, OX40, GITR, ICOS, 4-1BB, ADORA2A, B7-H3, B7-H4, BTLA, CTLA-4, IDO, KIR, LAG-3, PD-1, PD-L1, PD-L2, TIM-3, and VISTA. The immune checkpoint modulator may be an agonist or an antagonist of the immune checkpoint. In some embodiments, the immune checkpoint modulator is an immune checkpoint binding protein (e.g., an antibody, antibody Fab fragment, divalent antibody, antibody drug conjugate, scFv, fusion protein, bivalent antibody, or tetravalent antibody). In other embodiments, the immune checkpoint modulator is a small molecule. In a particular embodiment, the immune checkpoint modulator is an anti-PD1, anti-PD-L1, or anti-CTLA-4 binding protein, e.g., antibody or antibody fragment, e.g., antigen-binding fragment.

An “immunotherapeutic” as used herein refers to a pharmaceutically acceptable compound, composition or therapy that induces or enhances an immune response. Immunotherapeutics include, but are not limited to, immune checkpoint modulators, Toll-like receptor (TLR) agonists, cell-based therapies, cytokines and cancer vaccines.

As used herein, “oncological disorder” or “cancer” or “neoplasm” refer to all types of cancer or neoplasm found in humans, including, but not limited to: leukemias, lymphomas, melanomas, carcinomas and sarcomas. As used herein, the terms “oncological disorder”, “cancer,” and “neoplasm,” used interchangeably and in either the singular or plural form, refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but also cancer stem cells, as well as cancer progenitor cells or any cell derived from a cancer cell ancestor. This includes metastasized cancer cells, and in vitro cultures and cell lines derived from cancer cells.

Specific criteria for the staging of cancer are dependent on the specific cancer type based on tumor size, histological characteristics, tumor markers, and other criteria known by those of skill in the art. Generally, cancer stages can be described as follows: (i) Stage 0, Carcinoma in situ; (ii) Stage I, Stage II, and Stage III, wherein higher numbers indicate more extensive disease, including larger tumor size and/or spread of the cancer beyond the organ in which it first developed to nearby lymph nodes and/or tissues or organs adjacent to the location of the primary tumor; and (iii) Stage IV, wherein the cancer has spread to distant tissues or organs.

A “solid tumor” is a tumor that is detectable on the basis of tumor mass; e.g., by procedures such as CAT scan, MR imaging, X-ray, ultrasound or palpation, and/or which is detectable because of the expression of one or more cancer-specific antigens in a sample obtainable from a patient. The tumor does not need to have measurable dimensions.

A “subject” to be treated by the methods of the invention can mean either a human or non-human animal, preferably a mammal, more preferably a human. In certain embodiments, a subject has a detectable or diagnosed cancer prior to initiation of treatments using the methods of the invention. In certain embodiments, a subject has a detectable or diagnosed infection, e.g., chronic infection, prior to initiation of treatments using the methods of the invention. A “suicide gene” as used herein refers to a gene encoding a protein (e.g., an enzyme) that converts a nontoxic precursor of a drug into a cytotoxic compound.

“Cell turnover”, as used herein, refers to a dynamic process that reorders and disseminates the material within a cell and may ultimately result in cell death. Cell turnover includes the production and release from the cell of cell turnover factors.

“Cell turnover factors”, as used herein, are molecules and cell fragments produced by a cell undergoing cell turnover that are ultimately released from the cell and influence the biological activity of other cells. Cell turnover factors can include proteins, peptides, carbohydrates, lipids, nucleic acids, small molecules, and cell fragments (e.g. vesicles and cell membrane fragments).

A “cell turnover pathway gene”, as used herein, refers to a gene encoding a polypeptide that promotes, induces, or otherwise contributes to a cell turnover pathway.

“Thanotransmission”, as used herein, is communication between cells that is a result of activation of a cell turnover pathway in a target signaling cell, which signals a responding cell to undergo a biological response. Thanotransmission may be induced in a target signaling cell by modulation of cell turnover pathway genes in said cell through, for example, viral or other gene therapy delivery to the target signaling cell of genes that promote such pathways. Tables 2, 3, 4, and 6 describe exemplary genes or proteins capable of promoting various cell turnover pathways. The target signaling cell in which a cell turnover pathway has been thus activated may signal a responding cell through factors actively released by the signaling cell, or through intracellular factors of the signaling cell that become exposed to the responding cell during the cell turnover (e.g., cell death) of the signaling cell. In certain embodiments, the activated signaling cell promotes an immuno-stimulatory response (e.g., a pro-inflammatory response) in a responding cell (e.g., an immune cell).

The terms “polynucleotide that promotes thanotransmision” and “thanotransmission polynucleotide” are used interchangeably herein to refer to a polynucleotide whose expression in a target cell results in an increase in thanotransmission by the target cell. In some embodiments, the polynucleotide that promotes thanotransmission encodes a polypeptide that promotes thanotransmission; the terms “polypeptide that promotes thanotransmission” and “thanotransmission polypeptide” are used interchangeably herein, and refer to a polypeptide whose expression in a target cell increases thanotransmission by the target cell. In some embodiments, the polynucleotide that promotes thanotransmission reduces expression and/or activity in a target cell of a polypeptide that suppresses thanotransmission. For example, the polynucleotide that promotes thanotransmission may encode an RNA molecule that reduces expression and/or activity in a target cell of a polypeptide that suppresses thanotransmission.

“Therapeutically effective amount” means the amount of a compound that, when administered to a patient for treating a disease, is sufficient to effect such treatment for the disease. When administered for preventing a disease, the amount is sufficient to avoid or delay onset of the disease. The “therapeutically effective amount” will vary depending on the compound, the disease and its severity and the age, weight, etc., of the patient to be treated. A therapeutically effective amount need not be curative. A therapeutically effective amount need not prevent a disease or condition from ever occurring. Instead a therapeutically effective amount is an amount that will at least delay or reduce the onset, severity, or progression of a disease or condition.

As used herein, “treatment”, “treating” and cognates thereof refer to the medical management of a subject with the intent to improve, ameliorate, stabilize, prevent or cure a disease, pathological condition, or disorder. This term includes active treatment (treatment directed to improve the disease, pathological condition, or disorder), causal treatment (treatment directed to the cause of the associated disease, pathological condition, or disorder), palliative treatment (treatment designed for the relief of symptoms), preventative treatment (treatment directed to minimizing or partially or completely inhibiting the development of the associated disease, pathological condition, or disorder); and supportive treatment (treatment employed to supplement another therapy).

The term “variant” as used herein with reference to a polypeptide refers to a polypeptide that differs by at least one amino acid residue from a corresponding wild type polypeptide. In some embodiments, the variant polypeptide has at least one activity that differs from the corresponding naturally occurring polypeptide. The term “variant” as used herein with reference to a polynucleotide refers to a polynucleotide that differs by at least one nucleotide from a corresponding wild type polynucleotide. In some embodiments, a variant polypeptide or variant polynucleotide has at least 50%, 55%, 60%, 65%, 70%, 75%, 80%, 85%, 90%, 95%, 96%, 97%, 98%, or 99% sequence identity to the corresponding wild type polypeptide or polynucleotide and the polypeptide or encoded polypeptide differs by at least one amino acid residue.

### II. Cell Turnover Pathways

The viruses engineered to comprise one or more polynucleotides that promote thanotransmission, as provided herein, may be used to modulate cell turnover pathways in a target cell. For example, in some embodiments, infection of the target cell with the engineered virus induces an immuno-stimulatory cell turnover pathway in the target cell. Immuno-stimulatory cell turnover pathways are cell turnover pathways that, when activated in a cell, promote an immune-stimulatory response in a responding cell, such as an immune cell. Immuno-stimulatory cell turnover pathways include, but are not limited to, programmed necrosis (e.g., pyroptosis, necroptosis), apoptosis, e.g., extrinsic and/or intrinsic apoptosis, autophagy, ferroptosis, and combinations thereof.

### Programmed Necrosis

“Programmed necrosis” as used herein refers to a genetically controlled cell death with morphological features such as cellular swelling (oncosis), membrane rupture, and release of cellular contents, in contrast to the retention of membrane integrity that occurs during apoptosis. In some embodiments, the programmed necrosis is pyropotosis. In some embodiments, the programmed necrosis is necroptosis.

### Pyroptosis

“Pyroptosis” as used herein refers to the inherently inflammatory process of caspase 1-, caspase 4-, or caspase 5-dependent programmed cell death. The most distinctive biochemical feature of pyroptosis is the early, induced proximity-mediated activation of caspase-1. The pyroptotic activation of caspase-1, 4 or 5 can occur in the context of a multiprotein platform known as the inflammasome, which involves NOD-like receptors (NLRs) or other sensors such as the cytosolic DNA sensor absent in melanoma 2 (AIM2) that recruit the adaptor protein ASC that promotes caspase-1 activation. Caspases-4/5 may be directly activated by LPS. In both cases, active caspase-1 catalyzes the proteolytic maturation and release of pyrogenic interleukin-1β (IL-1β) and IL-18. Moreover, in some (but not all) instances, caspase activation induces cleavage and activation of the pore forming protein GSDM-D to drive membrane rupture and cell death. (See Galluzzi et al., 2018, Cell Death Differ. March; 25(3): 486-541.) In the methods of the present disclosure, pyroptosis may be induced in a target cell through contact or infection with a virus engineered to comprise one or more polynucleotides encoding a polypeptides that induces pyroptosis in the target cell. Polypeptides that may induce pyroptosis in a target cell include, but are not limited to, NLRs, ASC, GSDM-D, AIM2, and BIRC1.

Several methods are known in the art and may be employed for identifying cells undergoing pyroptosis and distinguishing from other types of cellular disassembly and/or cell death through detection of particular markers. Pyroptosis requires caspase-1, caspase-4, or caspase-5 activity and is usually accompanied by the processing of the pro-IL-1b and/or pro-IL-18, release of these mature cytokines, and membrane permeabilization by a caspase-1/4/5 cleavage fragment of GSDM-D.

Necroptosis

The term “necroptosis” as used herein refers to Receptor interacting protein kinase 1 and/or 3 (RIPK1- and/or RIPK3)/Mixed lineage kinase-like (MLKL)-dependent necrosis. Several triggers can induce necroptosis, including alkylating DNA damage, excitotoxins and the ligation of death receptors. For example, when caspases (and in particular caspase-8 or caspase-10) are inhibited by genetic manipulations (e.g., by gene knockout or RNA interference, RNAi) or blocked by pharmacological agents (e.g., chemical caspase inhibitors), RIPK3 phosphorylates MLKL leading to MLKL assembly into a membrane pore that ultimately activates the execution of necrotic cell death. See Galluzzi et al., 2018, Cell Death Differ. March; 25(3): 486-541, incorporated by reference herein in its entirety.

The same pathways that drive immunogenic apoptosis can activate RIPK3 but normally caspase 8 (and potentially caspase 10) suppresses RIPK3 activation. RIPK3 is typically only activated in situations of caspase 8 compromise. Viral proteins such as vICA or cellular mutants such as FADD dominant negative (DN) target caspase 8 pathways and unleash RIPK3 activity if RIPK3 is present. If RIPK3 is not present, then vICA or FADD-DN simply block apoptosis. Necroptosis is immunogenic because (a) membrane ruptures and (b) an inflammatory transcriptional program (e.g., NF-kB and IRF3) are concomitantly activated.

In the methods of the present disclosure, necroptosis may be induced in a target cell through contact or infection with a virus engineered to comprise one or more polynucleotides encoding a polypeptide that induces necroptosis in the target cell. Polypeptides that may induce necroptosis in a target cell include, but are not limited to, Toll-like receptor 3 (TLR3), TLR4, TIR Domain Containing Adaptor Protein (TIRAP), Toll/interleukin-1 receptor (TIR)-domain-containing adapter-inducing interferon-β (TRIF), Z-DNA-binding protein 1 (ZBP1), receptor-interacting serine/threonine-protein kinase 1 (RIPK1), receptor-interacting serine/threonine-protein kinase 3 (RIPK3), mixed lineage kinase domain like pseudokinase (MLKL), tumor necrosis factor receptor (TNFR), FS-7-associated surface antigen (FAS), TNF-related apoptosis inducing ligand receptor (TRAILR) and Tumor Necrosis Factor Receptor Type 1-Associated Death Domain Protein (TRADD).

Several methods are known in the art and may be employed for identifying cells undergoing necroptosis and distinguishing from other types of cellular disassembly and/or cell death through detection of particular markers. These markers include phosphorylation of RIPK1, RIPK3, and MLKL, which can be detected by antibodies that detect these post-translational modifications, typically by immunoblot or immunostaining of cells. Necroptosis can be distinguished from apoptosis and pyroptosis by the absence of caspase activation, rapid membrane permeabilization, MLKL relocalization to membranes, accumulation of RIPK3 and MLKL into detergent insoluble fractions, RIPK3/MLKL complex formation, and MLKL oligomerization. Necroptosis can be genetically and pharmacologically defined by requirement of both RIPK3 and MLKL as well as their activation.

### Apoptosis

Apoptosis, as used herein, refers to a type of programmed cell death characterized by specific morphological and biochemical changes of dying cells, including cell shrinkage, nuclear condensation and fragmentation, dynamic membrane blebbing and loss of adhesion to neighbors or to extracellular matrix (Nishida K, et al., (2008) Circ. Res. 103, 343-351). There are two basic apoptotic signaling pathways: the extrinsic and the intrinsic pathways (Verbrugge I, et al., (2010) Cell. 143:1192-2). The intrinsic apoptotic pathway is activated by various intracellular stimuli, including DNA damage, growth factor deprivation, and oxidative stress. The extrinsic pathway of apoptosis is initiated by the binding of death ligands to death receptors, followed by the assembly of the death-inducing signaling complex, which either activates downstream effector caspases to directly induce cell death or activate the mitochondria-mediated intrinsic apoptotic pathway (Verbrugge I, et al., (2010) Cell. 143:1192-2).

Extrinsic Apoptosis

The term ‘extrinsic apoptosis’ as used herein refers to instances of apoptotic cell death that are induced by extracellular stress signals which are sensed and propagated by specific transmembrane receptors. Extrinsic apoptosis can be initiated by the binding of ligands, such as FAS/CD95 ligand (FASL/CD95L), tumor necrosis factor α (TNFα), and TNF (ligand) superfamily, member 10 (TNFSF10, best known as TNF-related apoptosis inducing ligand, TRAIL), to various death receptors (i.e., FAS/CD95, TNFα receptor 1 (TNFR1), and TRAIL receptor (TRAILR)1-2, respectively). Alternatively, an extrinsic pro-apoptotic signal can be dispatched by the so-called ‘dependence receptors’, including netrin receptors (e.g., UNCSA-D and deleted in colorectal carcinoma, DCC), which only exert lethal functions when the concentration of their specific ligands falls below a critical threshold level. (See Galluzzi et al., 2018, Cell Death Differ. March; 25(3): 486-541, incorporated by reference herein in its entirety.)

In the methods of the present disclosure, extrinsic apoptosis may be induced in a target cell through contact or infection with a virus engineered to comprise one or more polynucleotides encoding a polypeptide that induces extrinsic apoptosis in the target cell. Polypeptides that may induce extrinsic apoptosis in a target cell include, but are not limited to, TNF, Fas ligand (FasL), TRAIL (and its cognate receptors), TRADD, Fas-associated protein with death domain (FADD), Transforming growth factor beta-activated kinase 1 (Tak1), Caspase-8, XIAP, BID, Caspase-9, APAF-1, CytoC, Caspase-3 and Caspase-7. Polypeptides that may inhibit extrinsic apoptosis in a target cell include Cellular Inhibitor of Apoptosis Protein 1 (cIAP1), cIAP2, Ikka and Ikkb Several methods are known in the art and may be employed for identifying cells undergoing apoptosis and distinguishing from other types of cellular disassembly and/or cell death through detection of particular markers. Apoptosis requires caspase activation and can be suppressed by inhibitors of caspase activation and/or prevention of death by the absence of caspases such as caspase-8 or caspase-9. Caspase activation systematically dismantles the cell by cleavage of specific substrates such as PARP and DFF45 as well as over 600 additional proteins. Apoptotic cell membranes initially remain intact with externalization of phosphotidyl-serine and concomitant membrane blebbing. Mitochondrial outer membranes are typically disrupted releasing into the cytosol proteins such as CytoC and HTRA2. Nuclear DNA is cleaved into discrete fragments that can be detected by assays known in the art.

### Autophagy

The term “autophagy”, as used herein, refers to an evolutionarily conserved catabolic process beginning with formation of autophagosomes, double membrane-bound structures surrounding cytoplasmic macromolecules and organelles, destined for recycling (Liu J J, et al., (2011) Cancer Lett. 300, 105-114). Autophagy is physiologically a cellular strategy and mechanism for survival under stress conditions. When over-activated under certain circumstances, excess autophagy results in cell death (Boya P, et al., (2013) Nat Cell Biol. (7):713-20).

In the methods of the present disclosure, autophagy may be induced in an immune cell through expression of one or more heterologous polynucleotides encoding a polypeptide that induces autophagy in the immune cell.

### Ferroptosis

The term “Ferroptosis”, as used herein, refers to a process of regulated cell death that is iron dependent and involves the production of reactive oxygen species. In some embodiments, ferroptosis involves the iron-dependent accumulation of lipid hydroperoxides to lethal levels. The sensitivity to ferroptosis is tightly linked to numerous biological processes, including amino acid, iron, and polyunsaturated fatty acid metabolism, and the biosynthesis of glutathione, phospholipids, NADPH, and Coenzyme Q10. Ferroptosis involves metabolic dysfunction that results in the production of both cytosolic and lipid ROS, independent of mitochondria but dependent on NADPH oxidases in some cell contexts (See, e.g., Dixon et al., 2012, Cell 149(5):1060-72, incorporated by reference herein in its entirety).

In the methods of the present disclosure, ferroptosis may be induced in a target cell through contact or infection with a virus engineered to comprise one or more polynucleotides that when expressed in a target cell reduce the expression or activity of a protein endogenous to the target cell that inhibits ferroptosis. Proteins that inhibit ferroptosis include, but are not limited to, FSP1, GPX4, and System XC.

Several methods are known in the art and may be employed for identifying cells undergoing ferroptosis and distinguishing from other types of cellular disassembly and/or cell death through detection of particular markers. (See, for example, Stockwell et al., 2017, Cell 171: 273-285, incorporated by reference herein in its entirety). For example, because ferroptosis may result from lethal lipid peroxidation, measuring lipid peroxidation provides one method of identifying cells undergoing iron-dependent cellular disassembly. C11-BODIPY and Liperfluo are lipophilic ROS sensors that provide a rapid, indirect means to detect lipid ROS (Dixon et al., 2012, Cell 149: 1060-1072). Liquid chromatography (LC)/tandem mass spectrometry (MS) analysis can also be used to detect specific oxidized lipids directly (Friedmann Angeli et al., 2014, Nat. Cell Biol. 16: 1180-1191; Kagan et al., 2017, Nat. Chem. Biol. 13: 81-90). Isoprostanes and malondialdehyde (MDA) may also be used to measure lipid peroxidation (Milne et al., 2007, Nat. Protoc. 2: 221-226; Wang et al., 2017, Hepatology 66(2): 449-465). Kits for measuring MDA are commercially available (Beyotime, Haimen, China).

Other useful assays for studying ferroptosis include measuring iron abundance and GPX4 activity. Iron abundance can be measured using inductively coupled plasma-MS or calcein AM quenching, as well as other specific iron probes (Hirayama and Nagasawa, 2017, J. Clin. Biochem. Nutr. 60: 39-48; Spangler et al., 2016, Nat. Chem. Biol. 12: 680-685), while GPX4 activity can be detected using phosphatidylcholine hydroperoxide reduction in cell lysates using LC-MS (Yang et al., 2014, Cell 156: 317-331). In addition, ferroptosis may be evaluated by measuring glutathione (GSH) content. GSH may be measured, for example, by using the commercially available GSH-Glo Glutathione Assay (Promega, Madison, WI).

Ferroptosis may also be evaluated by measuring the expression of one or more marker proteins. Suitable marker proteins include, but are not limited to, glutathione peroxidase 4 (GPX4), prostaglandin-endoperoxide synthase 2 (PTGS2), and cyclooxygenase-2 (COX-2). The level of expression of the marker protein or a nucleic acid encoding the marker protein may be determined using suitable techniques known in the art including, but not limited to polymerase chain reaction (PCR) amplification reaction, reverse-transcriptase PCR analysis, quantitative real-time PCR, single-strand conformation polymorphism analysis (SSCP), mismatch cleavage detection, heteroduplex analysis, Northern blot analysis, Western blot analysis, in situ hybridization, array analysis, deoxyribonucleic acid sequencing, restriction fragment length polymorphism analysis, and combinations or sub-combinations thereof.

### III. Viruses

In certain aspects the disclosure relates to a virus engineered to comprise one or more polynucleotides that promote thanotransmission by a target cell. Any virus that has the capacity to transfer a polynucleotide that promotes thanotransmission into a target cell may be used. For example, in some embodiments, the virus is capable of transporting a heterologous polynucleotide of at least 4, 5, 6, 7, 8, 9 or 10 kb into a target cell. In some embodiments, the virus is capable of transporting a heterologous polynucleotide of between 4-12 kb into a target cell. In some embodiments, the virus is cytolytic, i.e., capable of lysing the target cell. In some embodiments, the virus is oncolytic, i.e., a virus that preferentially infects and/or lyses cancer cells. In some embodiments, the virus preferentially infects the target cell. In some embodiments, the virus preferentially infects rapidly dividing cells (e.g. cancer cells). In some embodiments, the virus preferentially infects cancer cells.

The virus may be a DNA virus or an RNA virus (e.g. a retrovirus). In some embodiments, the virus is an RNA virus. In some embodiments, the virus is a DNA virus. In some embodiments, the DNA virus is a DNA replicative virus, e.g. a DNA replicative oncolytic virus.

In some embodiments, the virus is capable of reinfecting a host that was previously infected with the virus. This characteristic allows for multiple administrations of the virus to a subject. In some embodiments, the virus innately triggers Z-NA recognition.

In some embodiments, it is advantageous for the virus to comprise an inactivating mutation in one or more endogenous viral genes. In some embodiments, the inactivating mutation is in an endogenous viral gene that contributes to virulence of the virus (e.g. ICP34.5), such that the inactivating mutation decreases virulence. In some embodiments, the inactivating mutation is in an endogenous viral gene that restricts turnover of the infected cell (e.g. ICP6 in HSV; E3L in Vaccinia virus), such that the inactivating mutation facilitates or increases turnover of the cell upon infection. In some embodiments, inactivating mutations in viral genes may be combined with expression of additional polynucleotides or polypeptides that modulate virulence or cell turnover. For example, expression of a delta-Zα1 mutant form of Vaccinia virus E3L may be combined with full deletion of ICP34.5 to restore replicative capacity.

Examples of suitable viruses and endogenous viral genes that may be targeted for deactivation are provided in the table below.

TABLE 1
Exemplary viruses and viral genes targeted for mutation.

| Virus | Mutations |
|---|---|
| Adenovirus | Adenovirus Early Region 1A (E1A) |
| | Adenovirus Early Region 1B (E1B) |
| HSV-1 | ICP34.5 is mutated to limit neurovirulence |
| | ICP47 is mutated to augment antigen presentation in HSV-1 infected |
| | cells |
| | ICP6 mutation of the RHIM domain (e.g. a four amino acid change) |
| | mutations at the C-terminus of ICP6 that inhibit Caspase-8 binding |
| Vaccinia | Mutate the Za domain of E3L to prevent Zα-nucleic acid recognition by |
| virus | the innate immune system |

In some embodiments, the virus engineered to comprise one or more polynucleotides that promote thanotransmission is an adenovirus. In some embodiments, the adenovirus is adenovirus serotype 5 (Ad5). In some embodiments, the adenovirus is adenovirus serotype 19A (Ad19A). In some embodiments, the adenovirus is adenovirus serotype 26 (Ad26). An adenovirus of one serotype may be engineered to comprise a fiber protein from a different adenovirus serotype. For example, in some embodiments, Ad5 is engineered to substitute the fiber protein from adenovirus serotype 35 (Ad35). This chimeric virus is referred to as Ad5/F35. (See Yotnda et al., 2001, Gene Therapy 8: 930-937, which is incorporated by reference herein in its entirety.) In some embodiments, Ad5 is engineered to substitute the fiber protein from adenovirus serotype 3 (Ad3). This chimeric virus is referred to as Ad5/F3.

In some embodiments, the adenovirus comprises one or more mutations (e.g., one or more substitutions, additions or deletions) relative to a corresponding wildtype adenovirus. For example, in some embodiments, the adenovirus (e.g., Ad5 or Ad5/F35) comprises a deletion in the Adenovirus Early Region 1A (E1A). In some embodiments, the adenovirus (e.g., Ad5 or Ad5/F35) comprises a 24 bp deletion in E1A. This deletion makes viral replication specific to cells with an altered Rb pathway. In some embodiments, the adenovirus (e.g., Ad5 or Ad5/F35) comprises a deletion in the Adenovirus Early Region 1B (E1B). In some embodiments, the adenovirus (e.g., Ad5 or Ad5/F35) comprises a 827 bp deletion in E1B. This deletion allows the virus to replicate in cells with P53 alterations. In a particular embodiment, the adenovirus (e.g., Ad5 or Ad5/F35) comprises a 24 bp deletion in E1A and a 827 bp deletion in E1B. In some embodiments, the adenovirus (e.g., Ad5 or Ad5/F35) has an Arg-Gly-Asp (RGD)-motif engineered into the fiber-H loop. This modification makes the adenovirus use αvβ3 and αvβ5 integrins (which are expressed in cancer cells) to enter the cell. (See Reynolds et al., 1999, Gene Therapy 6: 1336-1339, which is incorporated by reference herein in its entirety.)

In some embodiments a polynucleotide as described herein (e.g., a polynucleotide encoding a thanotransmission polypeptide) may be inserted into the E1 region of the adenovirus, e.g. in E1A or E1B. For example, in some embodiments the E1 region is removed and replaced with the polynucleotide. The polynucleotde may be operably linked to a promoter as described herein, e.g., a promoter that is heterologous to the virus. In some embodiments, a polynucleotide as described herein (e.g., a polynucleotide encoding a thanotransmission polypeptide) may be inserted downstream of an endogenous viral promoter to drive expression of the polynucleotide. For example, in some embodiments, the polynucleotide is inserted into an adenovirus downstream of the strong viral L5 promotor. The L5 promoter confers expression concomitant with late viral gene expression.

In some particular embodiments, the virus is not an adenovirus. In some embodiments, the virus is not an adeno-associated virus (AAV). In some embodiments, the virus is not an adenovirus or an AAV. In a further particular embodiment, the virus does not comprise a polynucleotide encoding a synthetic multimerization domain, i.e. a non-naturally occurring domain that physically associates with other such domains with sufficient affinity such that the domains are held in proximity to one another. In some embodiments, the virus is not an adenovirus or AAV comprising a polynucleotide encoding a synthetic multimerization domain, i.e. a non-naturally occurring domain that physically associates with other such domains with sufficient affinity such that the domains are held in proximity to one another.

In some embodiments, the virus engineered to comprise one or more polynucleotides that promote thanotransmission is a herpes simplex virus (HSV), e.g. HSV1. In some embodiments, the HSV1 is selected from Kos, F1, MacIntyre, McKrae and related strains. The HSV may be defective in one or more genes selected from ICP6, ICP34.5, ICP47, UL24, UL55, and UL56. In a particular embodiment, the ICP34.5 encoding gene is replaced by a polynucleotide cassette comprising a US11 encoding gene operably linked to an immediate early (IE) promoter. In a further particular embodiment, the HSV comprises a ΔZα mutant form of a Vaccinia virus E3L gene.

In one embodiment, the HSV is defective in one or more functions of ICP6. For example, mutation of the ICP6 gene may result in different losses of function depending on the mutation. In some embodiments, the ICP6 comprises one or more mutations of the receptor-interacting protein homotypic interaction motif (RHIM) domain. In some embodiments, the ICP6 comprises one or more mutations at the C-terminus that inhibit caspase-8 binding. In some embodiments, the ICP6 comprises one or more mutations that reduces or eliminates ribonucleotide reductase (RR) activity.

In some embodiments, the HSV expresses the US11 gene as an immediate early gene. The US11 protein is required for protein translation regulation late in the viral life cycle. Immediate-early expression of US11 is able to compensate for a loss-of-function mutation in ICP34.5 and so to counteract the shutoff of protein synthesis in a mutant virus with a deletion of ICP34.5, resulting in a less attenuated virus.

In other embodiments, the virus belongs to the Poxviridae family, e.g. a virus selected from myxoma virus, Yaba-like disease virus, raccoonpox virus, orf virus and cowpox virus. In some embodiments, the virus belongs to the Chordopoxvirinae subfamily of the Poxviridae family. In some embodiments, the virus belongs to the Orthopoxvirus genus of the Chordopoxvirinae subfamily. In some embodiments, the virus belongs to the Vaccinia virus species of the Orthopoxvirus genus. In some embodiments, the Vaccinia virus is a strain selected from the group consisting of Dairenl, IHD-J, L-IPV, LC16M8, LC16MO, Lister, LIVP, Tashkent, WR 65-16, Wyeth, Ankara, Copenhagen, Tian Tan and WR.

In one embodiment, the Vaccinia virus is engineered to lack thymidine kinase (TK) activity. In one embodiment, the Vaccinia virus has an inactivating mutation or deletion in the J2R gene that reduces or eliminates TK activity. The J2R gene encodes a TK that forms part of the salvage pathway for pyrimidine deoxyribonucleotide synthesis. In some embodiments, the Vaccinia virus is engineered to lack ribonucleotide reductase (RR) activity. In some embodiments, the Vaccinia virus has an inactivating mutation or deletion in a gene selected from I4L and F4L gene that reduces or eliminates RR activity. Reductions in TK activity or RR activity increases replication of the virus in transformed cells (e.g. cancer cells).

Vaccinia virus encodes multiple proteins that interfere with apoptotic, necroptotic and pyroptotic signalling. For example, E3, which is encoded by the E3L gene, is an important interferon antagonist that also affects Vaccinia host range and contributes to virulence. E3 was characterized first as a 25-kDa dsRNA binding protein that antagonizes the anti-viral activity of the interferon-induced dsRNA binding protein PKR and possesses a C-terminal dsRNA binding domain. The N-terminal region of E3 forms a distinct domain that has similarity with Z-DNA binding proteins and both N- and C-terminal domains contribute to virus virulence. E3 was also described as an apoptosis inhibitor when HeLa cells infected with a mutant Vaccinia lacking the E3L gene resulted in rapid cell death. (See Veyer et al., 2017, Immunology Letters 186: 68-80.) Accordingly, in some embodiments, the Vaccinia virus is defective in the E3L gene. In some embodiments, the E3L gene has a mutation that results in induction of necroptosis upon infection of a cancer cell.

In some embodiments, the virus engineered to comprise one or more polynucleotides that promote thanotransmission is not a Vaccinia virus. In some particular embodiments, the virus engineered to comprise one or more polynucleotides that promote thanotransmission is not an adenovirus. In some embodiments, the virus engineered to comprise one or more polynucleotides that promote thanotransmission is not an adeno-associated virus (AAV). In some embodiments, the virus engineered to comprise one or more polynucleotides that promote thanotransmission is not an adenovirus or an AAV. In a further particular embodiment, the virus does not comprise a polynucleotide encoding a synthetic multimerization domain, i.e. a non-naturally occurring domain that physically associates with other such domains with sufficient affinity such that the domains are held in proximity to one another. In some embodiments, the virus is not an adenovirus or AAV comprising a polynucleotide encoding a synthetic multimerization domain, i.e. a non-naturally occurring domain that physically associates with other such domains with sufficient affinity such that the domains are held in proximity to one another.

In some embodiments, the virus (e.g. HSV) comprises a microRNA (miR) target sequence. The miR target sequence prevents viral pathogenesis in normal cells without impeding virus replication in tumor cells. The miR target sequence may be inserted into one or more viral gene loci, e.g. one or more viral genes required for replication of the virus in normal (e.g. non-cancerous) cells. An exemplary microRNA target sequence for inclusion in the virus is miR-124, which has particular application for neural applications. Other microRNA target sequences can alternatively be employed for protecting other types of tissues, and it is within the ordinary skill in the art to select a suitable microRNA target sequence to protect a desired tissue or cell type. For example, miR-122 and miR-199 are expressed in normal liver cells but not primary liver cancer; thus one or a combination of miR-122 and/or miR-199 microRNA target sequences can be employed in embodiments of the viruses for treatment of liver cancers. Similarly, target sequences for miR-128 and/or miR-137 microRNA can be employed in the virus for protection of normal brain. An exemplary microRNA target sequence can be the reverse complement of the microRNA.

In some embodiments, the microRNA target sequences are included in the 3′ untranslated region (“UTR) of an HSV gene, to silence that gene in the presence of the microRNA. Multiple copies (e.g. two copies, three copies, four copies, five copies, six copies, or more) of the microRNA target sequence may be inserted in tandem. The multiple copies of the micro-RNA target sequence may be separated by spacers of four or more nucleotides (e.g. eight or more nucleotides). Without wishing to be bound by theory, it is believed that greater spacing (e.g., larger than about 8 nucleotides) provides increased stability.

To assist in protecting non-cancerous cells from the lytic effect of HSV infection, the multiple copies of the microRNA target sequence are inserted in the 3′ UTR of an HSV gene that is essential for replication in non-cancerous cells, which are known to persons of ordinary skill. The site may be the 3′ UTR of the microRNA-targeted gene in its normal (or native) locus within the HSV genome. In a particular embodiment, the virus is an HSV that includes multiple copies of the microRNA target sequence inserted into the 3′UTR of the ICP4 gene, e.g. one or both copies of the ICP4 gene, in viruses that have both native copies of the ICP4 gene.

In certain embodiments, the genome of the virus contains a deletion of the internal repeat (joint) region comprising one copy each of the diploid genes ICP0, ICP34.5, LAT and ICP4 along with the promoter for the ICP47 gene. In other embodiments, instead of deleting the joint, the expression of genes in the joint region, particularly ICP0 and/or ICP47, can be silenced by deleting these genes or otherwise limited mutagenesis of them.

In some embodiments, the virus comprises a ligand specific for a molecule (e.g. a protein, lipid or carbohydrate) present on the surface of a target cell, e.g. a cancer cell. The ligand may be incorporated into a glycoprotein exposed on the viral surface (e.g. gD or gC of HSV) to facilitate targeting the desired cell with the ligand. For example, the ligand can be incorporated between residues 1 and 25 of gD. Exemplary ligands for targeting GBM and other cancer cells include those targeting EGFR and EGFRVIII, CD133, CXCR4, carcinoembryonic antigen (CEA), ClC-3/annexin-2/MMP-2, human transferrin receptor and EpCAM. The ligand may target such a receptor or cell-surface molecule, i.e., the ligand can be capable of specifically binding such receptor or cell-surface molecule. EGFR- and EGFRVIII-specific ligands, such as antibodies (e.g. single chain antibodies) and VHHs (single domain antibodies), have been described in the literature (Kuan et al. Int. J. Cancer, 88, 962-69 (2000); Wickstrand et al., Cancer Res., 55(14):3140-8 (1995); Omid far et al., Tumor Biology, 25:296-305 (2004); see also Uchidaetal. Molecular Therapy, 21:561-9 (2013); see also Braidwood et al., Gene Then, 15, 1579-92 (2008)).

The virus also or alternatively may be targeted by incorporating ligands into other cell-surface molecules or receptors that are not necessarily cancer-associated. For example, ligands can include binding domains from natural ligands (e.g., growth factors (such as EGF, which can target EGFR, NGF, which can target trkA and the like)), peptide or non-peptide hormones, peptides selecting for binding a target molecule (e.g., designed ankyrin repeat proteins (DARPins)), etc. The virus also can include a mutant form of gB and/or gD that facilitates vector entry though non-canonical receptors (and may also have such mutations in one or both of these genes within the HSV genome).

### IV. Virus Payloads

A virus of the present disclosure may be engineered to comprise one or more polynucleotides that promote thanotransmission of a target cell upon infection. For example, in some embodiments, the engineered virus comprises at least one polynucleotide encoding a polypeptide that promotes thanotransmission in the target cell. In some embodiments, the engineered virus comprises at least 2, 3, 4 or 5 polynucleotide sequences each encoding a polypeptide that promotes thanotransmission in a target cell. Exemplary polypeptides and polynucleotides that promote thanotransmission in a target cell are provided in Table 2A, Table 2B, Table 3, Table 4, Table 5 and Table 6 below.

In some embodiments, the polynucleotide comprised by the virus encodes a wild type protein. In some embodiments, the polynucleotide comprised by the virus encodes a biologically active fragment of a wild type protein, e.g. an N-terminal or C-terminal truncation of a wild type protein or another functional fragment or domain of a wild type protein. In some embodiments, the polynucleotide comprised by the virus encodes a protein or a functional fragment or domain thereof comprising one or more mutations. In some embodiments, the polynucleotide comprised by the virus encodes a human protein or functional fragment thereof, e.g. a human wild type protein or functional fragment thereof, or a variant of a human protein or functional fragment thereof.

TABLE 2A
Exemplary polypeptides that promote thanotransmission by a target cell. (Exemplary
Accession numbers for Pfam entries of death fold domains (e.g., death domain (PF00531),
and CARD (PF00619)) and TIR domains (PF01582) are provided. The remaining accession
numbers refer to polynucleotide sequences encoding the polypeptide.)

| Polypeptide | Accession No. |
|---|---|
| A death fold domain (e.g. a death domain, a pyrin domain, a Death Effector | PF00531 |
| Domain (DED), a C-terminal caspase recruitment domain (CARD), and | PF00619 |
| variants thereof) | |
| A death domain from Fas-associated protein with death domain protein | NP_003815 |
| (FADD-DD) | |
| A dominant negative mutant of FADD-DD | NP_003815 |
| myristolated FADD-DD (myr-FADD-DD) | NP_003815 |
| A death domain from Fas protein | NM_000043.6 |
| A death domain from Tumor necrosis factor receptor type 1-associated | NM_003789.4 |
| death domain protein (TRADD) | |
| A death domain of Tumor necrosis factor receptor type 1 protein (TNFR1) | NM_001065.4 |
| A pyrin domain from NLR Family Pyrin Domain Containing 3 (NLRP3) | NM_001127461 |
| A pyrin domain from apoptosis-associated speck-like protein (ASC) | NM_013258.5 |
| A DED from Fas-associated protein with death domain (FADD) | NP_003815 |
| A DED from caspase-8 | NM_001372051.1 |
| A DED from caspase-10 | NM_032977.4 |
| A CARD from RIP-associated ICH1/CED3-homologous protein (RAIDD), | NM_003805.5 |
| A CARD from apoptosis-associated speck-like protein (ASC) | NM_013258.5 |
| A CARD from mitochondrial antiviral-signaling protein (MAVS) | NM_020746.5 |
| A CARD from caspase-1 | NM_033292.4 |
| A Toll/interleukin-1 receptor (TIR) domain (e.g. a TIR domain from | PF01582 |
| Myeloid Differentiation Primary Response Protein 88 (MyD88), | |
| Toll/interleukin-1 receptor (TIR)-domain-containing adapter-inducing | |
| interferon-β (TRIF), Toll Like Receptor 3 (TLR3), Toll Like Receptor 4 | |
| (TLR4), TIR Domain Containing Adaptor Protein (TIRAP), or | |
| Translocating chain-associated membrane protein (TRAM)) | |
| Myeloid Differentiation Primary Response Protein 88 (MyD88) | NM_001172567.2 |
| Toll Like Receptor 3 (TLR3) | NM_003265.3 |
| Toll Like Receptor 4 (TLR4) | NM_138554.5 |
| TIR Domain Containing Adaptor Protein (TIRAP) | NM_001318777.2 |
| Translocating chain-associated membrane protein (TRAM) | NM_021649.7 |
| Fas-associated protein with death domain (FADD) | NP_003815 |
| Tumor necrosis factor receptor type 1-associated death domain (TRADD) | NM_003789.4 |
| inhibitor kBα (IkBα) | NM_020529.3 |
| Interleukin-1 receptor-associated kinase 1 (IRAK1) | NM_001569.4 |
| Granzyme A | NM_006144.4 |
| Receptor-interacting serine/threonine-protein kinase 1/3 (RIPK1 and | NM_003804.6 |
| RIPK3) | NM_006871.4 |
| Z-DNA-binding protein 1 (ZBP1) | NM_030776.3 |
| Mixed lineage kinase domain like pseudokinase (MLKL) | NM_152649.4 |
| Toll/interleukin-1 receptor (TIR)-domain-containing adapter-inducing | NM_174989.5 |
| interferon-β (TRIF) | |
| N-terminal truncation of TRIF that comprises only a TIR domain and a | NM_174989.5 |
| RHIM domain | |
| Interferon Regulatory Factor 3 (IRF3) | NM_001571.6 |
| Cellular FLICE (FADD-like IL-1β-converting enzyme)-inhibitory protein | NM_003879.7 |
| (c-FLIP) | |
| Gasdermin-A (GSDM-A), gasdermin-B (GSDM-B), gasdermin-C (GSDM- | NM_178171.5 |
| C), gasdermin-D (GSDM-D) and gasdermin-E (GSDM-E) | NM_001042471.2 |
| | NM_031415.3 |
| | NM_004403.3 |
| | NM_024736.7 |
| Apoptosis-associated speck-like protein (ASC) | NM_013258.5 |
| Apoptosis-associated speck-like protein containing C-terminal caspase | NM_013258.5 |
| recruitment domain (ASC-CARD) | |
| Tumor necrosis factor (TNF) | NM_000594.4 |
| FS-7-associated surface antigen (FAS) | NM_000043.6 |
| TNF-related apoptosis inducing ligand (TRAIL) | NM_003810.4 |
| TNF-related apoptosis inducing ligand receptor (TRAILR) | NM_003844.4 |
| Caspase-3, Caspase-7, Caspase-8 and Caspase-9 | NM_001372051.1 |
| | NM_004346.4 |
| | NM_001227.5 |
| | NM_001229.5 |
| Caspase-8 death domain (DD) | NM_001372051.1 |
| XIAP | NM_001167.4 |
| BID | NM_197966.3 |
| APAF-1 | NM_013229.3 |
| TRAF2, TRAF3, TRAF5, TRAF6 | HM991672.1 |
| | NG_027973 |
| | NM_004619.4 |
| | NM_145803.3 |
| CytoC | NM_018947 |
| Cellular Inhibitor of Apoptosis Protein 1 (cIAP1) | NM_001166.5 |
| Cellular Inhibitor of Apoptosis Protein 2 (cIAP2) | NM_001165.5 |
| Transforming growth factor beta-activated kinase 1 (Tak1) | NP_003179.1 |
| IKKα | NM_001278.5 |
| IKKβ | NM_001556.3 |
| Nemo | NM_001321396.3 |
| NLRs (e.g. NOD2) | NM_022162.3 |
| Absent in melanoma 2 (AIM2) | NM_004833.3 |
| vFLIP (ORF71/K13) from Kaposi sarcoma-associated herpesvirus (KSHV) | YP_001129429.1 |
| MC159L from Molluscum Contagiousum virus | QHW18671.1 |
| E8 from Equine Herpes Virus 2 | NP_042671.1 |
| vICA from Human cytomegalovirus (HCMV) or Murine cytomegalovirus | APA45801.1 |
| (MCMV) | |
| CrmA from Cow Pox virus | CAB5514194.1 |
| P35 from Autographa californica multicapsid nucleopolyhedrovirus | P08160.1 |
| (AcMNPV) | |

In some embodiments, the one or more polynucleotides that promote thanotransmission encode any one or more of the polypeptides listed in Table 2A or 2B (or polypeptides at least 85%, 87%, 90%, 95%, 97%, 98%, or 99% identical thereto), or encode any one of the polypeptide domains listed in Table 3 (or domains at least 85%, 87%, 90%, 95%, 97%, 98%, or 99% identical thereto). In some embodiments, the one or more polynucleotides that promote thanotransmission encode any one or more of receptor-interacting serine/threonine-protein kinase 3 (RIPK3), Z-DNA-binding protein 1 (ZBP1), mixed lineage kinase domain like pseudokinase (MLKL), Toll/interleukin-1 receptor (TIR)-domain-containing adapter-inducing interferon-β (TRIF), an N-terminal truncation of TRIF that comprises only a TIR domain and a RHIM domain, Interferon Regulatory Factor 3 (IRF3), a truncated Fas-associated protein with death domain (FADD), and Cellular FLICE (FADD-like IL-1β-converting enzyme)-inhibitory protein (c-FLIP). In some embodiments, the cFLIP is a human cFLIP. In some embodiments, the cFLIP is Caspase-8 and FADD Like Apoptosis Regulator (cFLAR). In some embodiment, the one or more polynucleotides that promote thanotransmission encode a polypeptide selected from the group consisting of gasdermin-A (GSDM-A), gasdermin-B (GSDM-B), gasdermin-C (GSDM-C), gasdermin-D (GSDM-D), gasdermin-E (GSDM-E), apoptosis-associated speck-like protein containing C-terminal caspase recruitment domain (ASC-CARD) with a dimerization domain, and mutants thereof.

In some embodiments, the one or more polynucleotides that promote thanotransmission encode a polypeptide selected from cIAP1, cIAP2, IKKa, IKKb, XIAP and Nemo. Although these polypeptides may suppress cell death, they may promote thanotransmission, for example, by promoting NF-kB activation. Accordingly in some embodiments, increasing expression of cIAP1, cIAP2, IKKa, IKKb, XIAP and/or Nemo in a target cell promotes thanotransmission by the target cell. In other embodiments, reducing expression of cIAP1, cIAP2, IKKa, IKKb, XIAP and/or Nemo in a target cell promotes thanotransmission by the target cell, for example, by attenuating their suppression of cell death, thereby promoting cell turnover.

In some embodiment, the polynucleotide that promotes thanotransmission encodes a death fold domain. Examples of death fold domains include, but are not limited to, a death domain, a pyrin domain, a Death Effector Domain (DED), a C-terminal caspase recruitment domain (CARD), and variants thereof.

In some embodiments, the death domain is selected from a death domain of Fas-associated protein with death domain (FADD), a death domain of the Fas receptor, a death domain of Tumor necrosis factor receptor type 1-associated death domain protein (TRADD), a death domain of Tumor necrosis factor receptor type 1 (TNFR1), and variants thereof. FADD is a 23 kDa protein, made up of 208 amino acids. It contains two main domains: a C terminal death domain (DD) and an N terminal death effector domain (DED). The domains are structurally similar to one another, with each consisting of 6 α-helices. The DD of FADD binds to receptors such as the Fas receptor at the plasma membrane via their DD. The DED of FADD binds to the DED of intracellular molecules such as procaspase 8. In some embodiments, the FADD-DD is a dominant negative mutant of FADD-DD, or a myristolated FADD-DD (myr-FADD-DD).

In some embodiments, the pyrin domain is from a protein selected from NLR Family Pyrin Domain Containing 3 (NLRP3) and apoptosis-associated speck-like protein (ASC).

In some embodiments, the Death Effector Domain (DED) is from a protein selected from Fas-associated protein with death domain (FADD), caspase-8 and caspase-10.

In some embodiments, the CARD is from a protein selected from RIP-associated ICH1/CED3-homologous protein (RAIDD), apoptosis-associated speck-like protein (ASC), mitochondrial antiviral-signaling protein (MAVS), caspase-1, and variants thereof.

In some embodiments, the polynucleotide that promotes thanotransmission encodes a TIR domain. In some embodiments, the polynucleotide that promotes thanotransmission encodes a protein comprising a TIR domain. The TIR domain may be from proteins including, but not limited to, Myeloid Differentiation Primary Response Protein 88 (MyD88), Toll/interleukin-1 receptor (TIR)-domain-containing adapter-inducing interferon-β (TRIF), Toll Like Receptor 3 (TLR3), Toll Like Receptor 4 (TLR4), TIR Domain Containing Adaptor Protein (TIRAP) and Translocating chain-associated membrane protein (TRAM).

In some embodiments, the polynucleotide that promotes thanotransmission is a viral gene. In some embodiments, the viral gene encodes a polypeptide selected from vFLIP (ORF71/K13) from Kaposi sarcoma-associated herpesvirus (KSHV), MC159L from Molluscum Contagiousum virus, E8 from Equine Herpes Virus 2, vICA from Human cytomegalovirus (HCMV) or Murine cytomegalovirus (MCMV), CrmA from Cow Pox virus, and P35 from Autographa californica multicapsid nucleopolyhedrovirus (AcMNPV).

Any of the polypeptides that promote thanotransmission by a target cell as described herein may be mutated to further enhance their ability to promote thanotransmission. For example, in some embodiments, the polynucleotide encoding ZBP1 comprises a deletion of receptor-interacting protein homotypic interaction motif (RHIM) C, a deletion of RHIM D, a deletion of RHIM B, and a deletion in the region encoding the N-terminus of the Zα1 domain.

In some embodiments, the one or more polynucleotides comprised by the virus that promote thanotransmission inhibits expression or activity of receptor-interacting serine/threonine-protein kinase 1 (RIPK1).

### Fusogenic Proteins

In one embodiment, the one or more polynucleotides comprised by the virus that promote thanotransmission encodes a fusogenic protein. The fusogenic protein may be any heterologous protein capable of promoting fusion of a cell infected with the virus to another cell. Fusogenic proteins are known in the art and are described, for example, in WO2017/118866, which is incorporated by reference herein in its entirety. Viruses expressing fusogenic proteins have been shown to enhance tumor cell killing relative to a virus that does not express the fusogenic protein. See WO2017/118866. Examples of fusogenic proteins include VSV-G, syncitin-1 (from human endogenous retrovirus-W (HERV-W)) or syncitin-2 (from HERVFRDE1), paramyxovirus SV5-F, measles virus-H, measles virus-F, RSV-F, the glycoprotein from a retrovirus or lentivirus, such as gibbon ape leukemia virus (GALV), murine leukemia virus (MLV), Mason-Pfizer monkey virus (MPMV) and equine infectious anemia virus (EIAV) with the R transmembrane peptide removed (R-versions). In one embodiment, the fusogenic protein is glycoprotein from gibbon ape leukemia virus (GALV) and has the R transmembrane peptide mutated or removed (GALV-R-). Exemplary fusogenic proteins are provided in Table 2B below.

TABLE 2B
Fusogenic proteins that promote thanotransmission by a target cell.

Indiana vesiculovirus G protein (VSV-G)
syncitin-1 (from human endogenous retrovirus-W (HERV-W)
syncitin-2 (from HERV-FRDE1)
paramyxovirus SV5-F protein
measles virus-H protein
measles virus-F protein
RSV-F protein
a glycoprotein from a retrovirus or lentivirus, such as gibbon ape
leukemia virus (GALV), murine leukemia virus (MLV), Mason-Pfizer
monkey virus (MPMV) and equine infectious anemia virus (EIAV) with
the R transmembrane peptide removed (R- versions)
a glycoprotein from gibbon ape leukemia virus (GALV) that has the R
transmembrane peptide mutated or removed (GALV-R-)

Chimeric Proteins that Promote Thanotransmission

In some embodiments, a polynucleotide that promotes thanotransmission may encode a chimeric protein. The chimeric protein may comprise any two or more of the domains listed in Table 3 below, e.g. 2, 3, 4 or 5 of the domains listed in Table 3. For example, in some embodiments, a polynucleotide that promotes thanotransmission encodes a chimeric protein comprising a TRIF TIR domain, a TRIF RHIM domain and ASC-CARD. This chimeric protein would recruit caspase-1 and activate pyroptosis. In some embodiments, the chimeric protein comprises a ZBP1 Za2 domain and ASC-CARD. This chimeric protein is expected to activate pyroptosis. In some embodiments, the chimeric protein comprises a RIPK3 RHIM domain and a caspase Large subunit/Small subunit (L/S) domain. This chimeric protein would drive constitutive activation of the caspase, leading to different types of cell death depending on the caspase L/S domain selected, as shown in Table 3. In some embodiments, the chimeric protein comprises a TRIF TIR domain, a TRIF RHIM domain and a FADD death domain (FADD-DD). This chimeric protein is expected to block apoptosis but induce necroptosis. In some embodiments, the chimeric protein comprises inhibitor kBα super-repressor (IkBαSR) and the caspase-8 DED domain. This chimeric protein is expected to inhibit NF-kB and induce apoptosis.

TABLE 3
Polypeptide domains that promote thanotransmission. (Abbreviations shown
are death domain (DD), death effector domain (DED), Caspase Recruitment
Domain (CARD), and Large subunit/Small subunit (L/S). The approximate size
of the polynucleotide encoding the polypeptide domain is indicated.)

| | Approximate Size of | |
|---|---|---|
| Domain | Polynucleotide (bp) | Expected Outcome |
| ZBP1-RHIMA | 50-150, e.g., 100 | Necroptosis |
| TRIF-RHIM | 50-150, e.g., 100 | Necroptosis |
| TRIF-TIR | 400-700 | Inhibit TLR; Induce IRF3 |
| RIPK3-RHIM | 50-150, e.g., 100 | Necroptosis |
| MyD88-DD | 250-400 | Inhibit IL-1R/TLR |
| MyD88-TIR | 400-700 | Inhibit IL-1R/TLR |
| FADD-DD | 250-400 | Block Extrinsic Apoptosis |
| FADD-DED | 250-400 | Induce Extrinsic Apoptosis |
| TRADD-DD | 250-400 | Inhibit/Induce Extrinsic Apoptosis |
| FAS-DD | 250-400 | Induce Extrinsic Apoptosis |
| TNFR-DD | 250-400 | Induce Extrinsic Apoptosis |
| Caspase-8-CARD | 250-400 | Induce Extrinsic Apoptosis |
| Caspase-8-L/S | 250-400 | Induce Extrinsic Apoptosis |
| Caspase-1-CARD | 250-400 | Pyroptosis |
| Caspase-1-L/S | 250-400 | Pyroptosis |
| Caspase-9-CARD | 250-400 | Intrinsic Apoptosis |
| Caspase9-L/S | 250-400 | Intrinsic Apoptosis |
| RIPK1 kinase domain | 550-800 | Induce necroptosis |
| RIPK3 kinase domain | 550-800 | Induce Necroptosis |
| MLKL pseudokinase | 550-800 | Induce Necroptosis; Inhibit Necroptosis |
| domain | | |

In some embodiments, the virus is engineered to comprises only one polynucleotide that promotes thanotransmission. In some embodiments, this single polynucleotide that promotes thanotransmission encodes only one thanotransmission polypeptide or domain thereof. In other embodiments, the virus is engineered to comprise one or more polynucleotides that promote thanotransmission that encode two or more different thanotransmission polypeptides, or domains thereof. In some embodiments, the two or more thanotransmission polypeptides are selected from the group consisting of TRADD, TRAF2, TRAF6, cIAP1, cIAP2, XIAP, NOD2, MyD88, TRAM, HOIL, HOIP, Sharpin, IKKg, IKKa, IKKb, RelA, MAVS, RIGI, MDA5, Tak1, TBK1, IKKe, IRF3, IRF7, IRF1, TRAF3, a Caspase, FADD, TNFR1, TRAILR1, TRAILR2, FAS, Bax, Bak, Bim, Bid, Noxa, Puma, TRIF, ZBP1, RIPK1, RIPK3, MLKL, Gasdermin A, Gasdermin B, Gasdermin C, Gasdermin D, Gasdermin E, a tumor necrosis factor receptor superfamily (TNFSF) protein, variants thereof, and functional fragments thereof.

Suitable caspases include caspase-1, caspase-2, caspase-2, caspase-3, caspase-4, caspase-5, caspase-6, caspase-7, caspase-8, caspase-9, caspase-10, caspase-11 and caspase-12.

Exemplary TNFSF proteins are provided in Table 4 below.

TABLE 4
Exemplary TNFSF proteins. (Adapted from Locksley et al., 2001, Cell. 104
(4): 487-501, which is incorporated by reference herein in its entirety.)

| Type | Protein | Synonyms | Gene | Ligand(s) |
|---|---|---|---|---|
| 1 | Tumor necrosis factor | CD120a | TNFRSF1A | TNF (cachectin) |
| | receptor 1 | | | |
| 1 | Tumor necrosis factor | CD120b | TNFRSF1B | TNF (cachectin) |
| | receptor 2 | | | |
| 3 | Lymphotoxin beta | CD18 | LTBR | Lymphotoxin beta (TNF- |
| | receptor | | | C) |
| 4 | OX40 | CD134 | TNFRSF4 | OX40L |
| 5 | CD40 | Bp50 | CD40 | CD154 |
| 6 | Decoy receptor 3 | TR6, M68 | TNFRSF6B | FasL, LIGHT, TL1A |
| 6 | Fas receptor | Apo-1, CD95 | FAS | FasL |
| 7 | CD27 | S152, Tp55 | CD27 | CD70, Siva |
| 8 | CD30 | Ki-1, TNR8 | TNFRSF8 | CD153 |
| 9 | 4-1BB | CD137 | TNFRSF9 | 4-1BB ligand |
| 10 | Death receptor 4 | TRAILR1, Apo-2, | TNFRSF10A | TRAIL |
| | | CD261 | | |
| 10 | Death receptor 5 | TRAILR2, CD262 | TNFRSF10B | TRAIL |
| 10 | Decoy receptor 1 | TRAILR3, LIT, TRID, | TNFRSF10C | TRAIL |
| | | CD263 | | |
| 10 | Decoy receptor 2 | TRAILR4, TRUNDD, | TNFRSF10D | TRAIL |
| | | CD264 | | |
| 11 | Osteoprotegerin | OCIF, TR1 | TNFRSF11B | RANKL |
| 11 | RANK | CD265 | TNFRSF11A | RANKL |
| 12 | TWEAK receptor | Fn14, CD266 | TNFRSF12A | TWEAK |
| 13 | BAFF receptor | CD268 | TNFRSF13C | BAFF |
| 13 | TACI | IGAD2, CD267 | TNFRSF13B | APRIL, BAFF, CAMLG |
| 14 | Herpesvirus entry | ATAR, TR2, CD270 | TNFRSF14 | LIGHT |
| | mediator | | | |
| 16 | Nerve growth factor | p75NTR, CD271 | NGFR | NGF, BDNF, NT-3, NT- |
| | receptor | | | 4 |
| 17 | B-cell maturation antigen | TNFRSF13A, CD269 | TNFRSF17 | BAFF |
| 18 | Glucocorticoid-induced | AITR, CD357 | TNFRSF18 | GITR ligand |
| | TNFR-related | | | |
| 19 | TROY | TAJ, TRADE | TNFRSF19 | unknown |
| 21 | Death receptor 6 | CD358 | TNFRSF21 | unknown |
| 25 | Death receptor 3 | Apo-3, TRAMP, | TNFRSF25 | TL1A |
| | | LARD, WS-1 | | |
| 27 | Ectodysplasin A2 receptor | XEDAR | EDA2R | EDA-A2 |

Exemplary polynucleotide sequences encoding the thanotransmission polypeptides of the disclosure are provided in Table 5 below. It will be understood that any other polynucleotide sequences that encode the thanotransmission polypeptides disclosed herein, including the polypeptides encoded by the genes listed in Table 5, (or encode polypeptides at least 85%, 87%, 90%, 95%, 97%, 98%, or 99% identical thereto) can be used in the methods and compositions described herein.

TABLE 5
Exemplary polynucleotide sequences encoding
thanotransmission polypeptides

| Gene Name: | Accession No.: |
|---|---|
| TRADD | NM_003789.4 |
| TRAF2 | HM991672.1 |
| TRAF3 | NG_027973 |
| TRAF6 | NM_145803.3 |
| cIAP1 | NM_001166.5 |
| cIAP2 | NM_001165.5 |
| XIAP | NM_001167.4 |
| NOD2 | NM_022162.3 |
| MyD88 | NM_001172567.2 |
| TRAM | NM_021649.7 |
| HOIP | AB265810 |
| HOIL | AB265810.1 |
| Sharpin | NM_017999.5 |
| IKKg | NM_001321396.3 |
| IKKa | NM_001278.5 |
| IKKb | NM_001556.3 |
| RelA | NM_021975.4 |
| MAVS | NM_020746.5 |
| RIGI | NM_014314.4 |
| MDA5 | NM_022168.4 |
| TAK1 | NM_079356.3 |
| TBK1 | NM_013254.4 |
| IKKe | NM_014002.4 |
| IRF3 | NM_001571.6 |
| IRF7 | NM_001572.5 |
| IRF1 | NM_002198.3 |
| TNFR1 | NM_001065.4 |
| TRAILR1 | NM_003844.4 |
| TRAILR2 | NM_003842.5 |
| FAS | NM_000043.6 |
| Bax | NM_138761.4 |
| Bak | NM_001188.4 |
| Bim | NM_138621.5 |
| Bid | NM_197966.3 |
| Noxa | NM_001382616.1 |
| Puma | NM_001127240.3 |
| TRIF | NM_174989.5 |
| ZBP1 | NM_030776.3 |
| RIPK3 | NM_006871.4 |
| RIPK1 | NM_003804.6 |
| MLKL | NM_152649.4 |
| GSDME | NM_004403.3 |
| GSDMD | NM_024736.7 |
| Caspase-8 | NM_001372051.1 |
| Caspase-10 | NM_032977.4 |

The two or more thanotransmission polypeptides may be expressed as separate polypeptides, or they may be comprised within a chimeric protein. In some embodiments, at least one of the polynucleotides that promote thanotransmision is transcribed as a single transcript that encodes the two or more thanotransmission polypeptides.

The thanotransmission polypeptides described herein may promote thanotransmission through various mechanisms, including but not limited to activation of NF-kB, activation of IRF3 and/or IRF7, promotion of apoptosis, and promotion of programmed necrosis (e.g., necroptosis or pyroptosis). When combinations of two or more thanotransmission polypeptides are used, each of the two or more thanotransmission polypeptides may promote thanotransmission through similar mechanisms, or through different mechanisms. For example, in some embodiments, at least two of the thanotransmission polypeptides encoded by the one or more polynucleotides activate NF-kB. In some embodiments, at least two of the thanotransmission polypeptides encoded by the one or more polynucleotides activate IRF3 and/or IRF7. In some embodiments, at least two of the thanotransmission polypeptides encoded by the one or more polynucleotides promote apoptosis. In some embodiments, at least two of the thanotransmission polypeptides encoded by the one or more polynucleotides promote programmed necrosis (e.g., necroptosis or pyroptosis).

When the two or more thanotransmission polypeptides promote thanotransmission through different mechanisms, various combinations of mechanisms may be used. For example, in some embodiments, at least one of the thanotransmission polypeptides encoded by the one or more thanotransmission polynucleotides activates NF-kB, and at least one of the thanotransmission polypeptides encoded by the one or more polynucleotides activates IRF3 and/or IRF7. In some embodiments, at least one of the thanotransmission polypeptides encoded by the one or more polynucleotides activates NF-kB, and at least one of the thanotransmission polypeptides encoded by the one or more polynucleotides promotes apoptosis. In some embodiments, at least one of the thanotransmission polypeptides encoded by the one or more polynucleotides activates NF-kB, and at least one of the thanotransmission polypeptides encoded by the one or more polynucleotides promotes programmed necrosis (e.g., necroptosis or pyroptosis). In some embodiments, at least one of the thanotransmission polypeptides encoded by the one or more polynucleotides activates IRF3 and/or IRF7, and at least one of the thanotransmission polypeptides encoded by the one or more polynucleotides promotes apoptosis. In some embodiments, at least one of the thanotransmission polypeptides encoded by the one or more thanotransmission polynucleotides activates IRF3 and/or IRF7, and at least one of the thanotransmission polypeptides encoded by the one or more polynucleotides promotes programmed necrosis (e.g., necroptosis or pyroptosis). In some embodiments, at least one of the thanotransmission polypeptides encoded by the one or more polynucleotides promotes apoptosis, and at least one of the thanotransmission polypeptides encoded by the one or more thanotransmission polynucleotides promotes programmed necrosis (e.g., necroptosis or pyroptosis).

In some embodiments, the thanotransmission polypeptide that activates NF-kB is selected from the group consisting of TRIF, TRADD, TRAF2, TRAF6, cIAP1, cIAP2, XIAP, NOD2, MyD88, TRAM, HOIL, HOIP, Sharpin, IKKg, IKKa, IKKb, RelA, MAVS, RIGI, MDA5, Tak1, a TNFSF protein, and functional fragments and variants thereof. In some embodiments, the thanotransmission polypeptide that activates IRF3 and/or IRF7 is selected from the group consisting of TRIF, MyD88, MAVS, TBK1, IKKe, IRF3, IRF7, IRF1, TRAF3 and functional fragments and variants thereof. In some embodiments, the thanotransmission polypeptide that promotes apoptosis is selected from the group consisting of TRIF, RIPK1, Caspase, FADD, TRADD, TNFR1, TRAILR1, TRAILR2, FAS, Bax, Bak, Bim, Bid, Noxa, Puma, and functional fragments and variants thereof. In some embodiments, the thanotransmission polypeptide that promotes programmed necrosis (e.g., necroptosis or pyroptosis) is selected from the group consisting of ZBP1, RIPK1, RIPK3, MLKL, a Gasdermin, and functional fragments and variants thereof.

In some embodiments, the combination of thanotransmission polypeptides is selected from TRADD and TRAF2, TRADD and TRAF6, TRADD and cIAP1, TRADD and cIAP2, TRADD and XIAP, TRADD and NOD2, TRADD and MyD88, TRADD and TRAM, TRADD and HOIL, TRADD and HOIP, TRADD and Sharpin, TRADD and IKKg, TRADD and IKKa, TRADD and IKKb, TRADD and RelA, TRADD and MAVS, TRADD and RIGI, TRADD and MDA5, TRADD and Tak1, TRADD and TBK1, TRADD and IKKe, TRADD and IRF3, TRADD and IRF7, TRADD and IRF1, TRADD and TRAF3, TRADD and a Caspase, TRADD and FADD, TRADD and TNFR1, TRADD and TRAILR1, TRADD and TRAILR2, TRADD and FAS, TRADD and Bax, TRADD and Bak, TRADD and Bim, TRADD and Bid, TRADD and Noxa, TRADD and Puma, TRADD and TRIF, TRADD and ZBP1, TRADD and RIPK1, TRADD and RIPK3, TRADD and MLKL, TRADD and Gasdermin A, TRADD and Gasdermin B, TRADD and Gasdermin C, TRADD and Gasdermin D, TRADD and Gasdermin E, TRAF2 and TRAF6, TRAF2 and cIAP1, TRAF2 and cIAP2, TRAF2 and XIAP, TRAF2 and NOD2, TRAF2 and MyD88, TRAF2 and TRAM, TRAF2 and HOIL, TRAF2 and HOIP, TRAF2 and Sharpin, TRAF2 and IKKg, TRAF2 and IKKa, TRAF2 and IKKb, TRAF2 and RelA, TRAF2 and MAVS, TRAF2 and RIGI, TRAF2 and MDA5, TRAF2 and Tak1, TRAF2 and TBK1, TRAF2 and IKKe, TRAF2 and IRF3, TRAF2 and IRF7, TRAF2 and IRF1, TRAF2 and TRAF3, TRAF2 and a Caspase, TRAF2 and FADD, TRAF2 and TNFR1, TRAF2 and TRAILR1, TRAF2 and TRAILR2, TRAF2 and FAS, TRAF2 and Bax, TRAF2 and Bak, TRAF2 and Bim, TRAF2 and Bid, TRAF2 and Noxa, TRAF2 and Puma, TRAF2 and TRIF, TRAF2 and ZBP1, TRAF2 and RIPK1, TRAF2 and RIPK3, TRAF2 and MLKL, TRAF2 and Gasdermin A, TRAF2 and Gasdermin B, TRAF2 and Gasdermin C, TRAF2 and Gasdermin D, TRAF2 and Gasdermin E, TRAF6 and cIAP1, TRAF6 and cIAP2, TRAF6 and XIAP, TRAF6 and NOD2, TRAF6 and MyD88, TRAF6 and TRAM, TRAF6 and HOIL, TRAF6 and HOIP, TRAF6 and Sharpin, TRAF6 and IKKg, TRAF6 and IKKa, TRAF6 and IKKb, TRAF6 and RelA, TRAF6 and MAVS, TRAF6 and RIGI, TRAF6 and MDA5, TRAF6 and Tak1, TRAF6 and TBK1, TRAF6 and IKKe, TRAF6 and IRF3, TRAF6 and IRF7, TRAF6 and IRF1, TRAF6 and TRAF3, TRAF6 and a Caspase, TRAF6 and FADD, TRAF6 and TNFR1, TRAF6 and TRAILR1, TRAF6 and TRAILR2, TRAF6 and FAS, TRAF6 and Bax, TRAF6 and Bak, TRAF6 and Bim, TRAF6 and Bid, TRAF6 and Noxa, TRAF6 and Puma, TRAF6 and TRIF, TRAF6 and ZBP1, TRAF6 and RIPK1, TRAF6 and RIPK3, TRAF6 and MLKL, TRAF6 and Gasdermin A, TRAF6 and Gasdermin B, TRAF6 and Gasdermin C, TRAF6 and Gasdermin D, TRAF6 and Gasdermin E, cIAP1 and cIAP2, cIAP1 and XIAP, cIAP1 and NOD2, cIAP1 and MyD88, cIAP1 and TRAM, cIAP1 and HOIL, cIAP1 and HOIP, cIAP1 and Sharpin, cIAP1 and IKKg, cIAP1 and IKKa, cIAP1 and IKKb, cIAP1 and RelA, cIAP1 and MAVS, cIAP1 and RIGI, cIAP1 and MDA5, cIAP1 and Tak1, cIAP1 and TBK1, cIAP1 and IKKe, cIAP1 and IRF3, cIAP1 and IRF7, cIAP1 and IRF1, cIAP1 and TRAF3, cIAP1 and a Caspase, cIAP1 and FADD, cIAP1 and TNFR1, cIAP1 and TRAILR1, cIAP1 and TRAILR2, cIAP1 and FAS, cIAP1 and Bax, cIAP1 and Bak, cIAP1 and Bim, cIAP1 and Bid, cIAP1 and Noxa, cIAP1 and Puma, cIAP1 and TRIF, cIAP1 and ZBP1, cIAP1 and RIPK1, cIAP1 and RIPK3, cIAP1 and MLKL, cIAP1 and Gasdermin A, cIAP1 and Gasdermin B, cIAP1 and Gasdermin C, cIAP1 and Gasdermin D, cIAP1 and Gasdermin E, cIAP2 and XIAP, cIAP2 and NOD2, cIAP2 and MyD88, cIAP2 and TRAM, cIAP2 and HOIL, cIAP2 and HOIP, cIAP2 and Sharpin, cIAP2 and IKKg, cIAP2 and IKKa, cIAP2 and IKKb, cIAP2 and RelA, cIAP2 and MAVS, cIAP2 and RIGI, cIAP2 and MDA5, cIAP2 and Tak1, cIAP2 and TBK1, cIAP2 and IKKe, cIAP2 and IRF3, cIAP2 and IRF7, cIAP2 and IRF1, cIAP2 and TRAF3, cIAP2 and a Caspase, cIAP2 and FADD, cIAP2 and TNFR1, cIAP2 and TRAILR1, cIAP2 and TRAILR2, cIAP2 and FAS, cIAP2 and Bax, cIAP2 and Bak, cIAP2 and Bim, cIAP2 and Bid, cIAP2 and Noxa, cIAP2 and Puma, cIAP2 and TRIF, cIAP2 and ZBP1, cIAP2 and RIPK1, cIAP2 and RIPK3, cIAP2 and MLKL, cIAP2 and Gasdermin A, cIAP2 and Gasdermin B, cIAP2 and Gasdermin C, cIAP2 and Gasdermin D, cIAP2 and Gasdermin E, XIAP and NOD2, XIAP and MyD88, XIAP and TRAM, XIAP and HOIL, XIAP and HOIP, XIAP and Sharpin, XIAP and IKKg, XIAP and IKKa, XIAP and IKKb, XIAP and RelA, XIAP and MAVS, XIAP and RIGI, XIAP and MDA5, XIAP and Tak1, XIAP and TBK1, XIAP and IKKe, XIAP and IRF3, XIAP and IRF7, XIAP and IRF1, XIAP and TRAF3, XIAP and a Caspase, XIAP and FADD, XIAP and TNFR1, XIAP and TRAILR1, XIAP and TRAILR2, XIAP and FAS, XIAP and Bax, XIAP and Bak, XIAP and Bim, XIAP and Bid, XIAP and Noxa, XIAP and Puma, XIAP and TRIF, XIAP and ZBP1, XIAP and RIPK1, XIAP and RIPK3, XIAP and MLKL, XIAP and Gasdermin A, XIAP and Gasdermin B, XIAP and Gasdermin C, XIAP and Gasdermin D, XIAP and Gasdermin E, NOD2 and MyD88, NOD2 and TRAM, NOD2 and HOIL, NOD2 and HOIP, NOD2 and Sharpin, NOD2 and IKKg, NOD2 and IKKa, NOD2 and IKKb, NOD2 and RelA, NOD2 and MAVS, NOD2 and RIGI, NOD2 and MDA5, NOD2 and Tak1, NOD2 and TBK1, NOD2 and IKKe, NOD2 and IRF3, NOD2 and IRF7, NOD2 and IRF1, NOD2 and TRAF3, NOD2 and a Caspase, NOD2 and FADD, NOD2 and TNFR1, NOD2 and TRAILR1, NOD2 and TRAILR2, NOD2 and FAS, NOD2 and Bax, NOD2 and Bak, NOD2 and Bim, NOD2 and Bid, NOD2 and Noxa, NOD2 and Puma, NOD2 and TRIF, NOD2 and ZBP1, NOD2 and RIPK1, NOD2 and RIPK3, NOD2 and MLKL, NOD2 and Gasdermin A, NOD2 and Gasdermin B, NOD2 and Gasdermin C, NOD2 and Gasdermin D, NOD2 and Gasdermin E, MyD88 and TRAM, MyD88 and HOIL, MyD88 and HOIP, MyD88 and Sharpin, MyD88 and IKKg, MyD88 and IKKa, MyD88 and IKKb, MyD88 and RelA, MyD88 and MAVS, MyD88 and RIGI, MyD88 and MDA5, MyD88 and Tak1, MyD88 and TBK1, MyD88 and IKKe, MyD88 and IRF3, MyD88 and IRF7, MyD88 and IRF1, MyD88 and TRAF3, MyD88 and a Caspase, MyD88 and FADD, MyD88 and TNFR1, MyD88 and TRAILR1, MyD88 and TRAILR2, MyD88 and FAS, MyD88 and Bax, MyD88 and Bak, MyD88 and Bim, MyD88 and Bid, MyD88 and Noxa, MyD88 and Puma, MyD88 and TRIF, MyD88 and ZBP1, MyD88 and RIPK1, MyD88 and RIPK3, MyD88 and MLKL, MyD88 and Gasdermin A, MyD88 and Gasdermin B, MyD88 and Gasdermin C, MyD88 and Gasdermin D, MyD88 and Gasdermin E, TRAM and HOIL, TRAM and HOIP, TRAM and Sharpin, TRAM and IKKg, TRAM and IKKa, TRAM and IKKb, TRAM and RelA, TRAM and MAVS, TRAM and RIGI, TRAM and MDA5, TRAM and Tak1, TRAM and TBK1, TRAM and IKKe, TRAM and IRF3, TRAM and IRF7, TRAM and IRF1, TRAM and TRAF3, TRAM and a Caspase, TRAM and FADD, TRAM and TNFR1, TRAM and TRAILR1, TRAM and TRAILR2, TRAM and FAS, TRAM and Bax, TRAM and Bak, TRAM and Bim, TRAM and Bid, TRAM and Noxa, TRAM and Puma, TRAM and TRIF, TRAM and ZBP1, TRAM and RIPK1, TRAM and RIPK3, TRAM and MLKL, TRAM and Gasdermin A, TRAM and Gasdermin B, TRAM and Gasdermin C, TRAM and Gasdermin D, TRAM and Gasdermin E, HOIL and HOIP, HOIL and Sharpin, HOIL and IKKg, HOIL and IKKa, HOIL and IKKb, HOIL and RelA, HOIL and MAVS, HOIL and RIGI, HOIL and MDA5, HOIL and Tak1, HOIL and TBK1, HOIL and IKKe, HOIL and IRF3, HOIL and IRF7, HOIL and IRF1, HOIL and TRAF3, HOIL and a Caspase, HOIL and FADD, HOIL and TNFR1, HOIL and TRAILR1, HOIL and TRAILR2, HOIL and FAS, HOIL and Bax, HOIL and Bak, HOIL and Bim, HOIL and Bid, HOIL and Noxa, HOIL and Puma, HOIL and TRIF, HOIL and ZBP1, HOIL and RIPK1, HOIL and RIPK3, HOIL and MLKL, HOIL and Gasdermin A, HOIL and Gasdermin B, HOIL and Gasdermin C, HOIL and Gasdermin D, HOIL and Gasdermin E, HOIP and Sharpin, HOIP and IKKg, HOIP and IKKa, HOIP and IKKb, HOIP and RelA, HOIP and MAVS, HOIP and RIGI, HOIP and MDA5, HOIP and Tak1, HOIP and TBK1, HOIP and IKKe, HOIP and IRF3, HOIP and IRF7, HOIP and IRF1, HOIP and TRAF3, HOIP and a Caspase, HOIP and FADD, HOIP and TNFR1, HOIP and TRAILR1, HOIP and TRAILR2, HOIP and FAS, HOIP and Bax, HOIP and Bak, HOIP and Bim, HOIP and Bid, HOIP and Noxa, HOIP and Puma, HOIP and TRIF, HOIP and ZBP1, HOIP and RIPK1, HOIP and RIPK3, HOIP and MLKL, HOIP and Gasdermin A, HOIP and Gasdermin B, HOIP and Gasdermin C, HOIP and Gasdermin D, HOIP and Gasdermin E, Sharpin and IKKg, Sharpin and IKKa, Sharpin and IKKb, Sharpin and RelA, Sharpin and MAVS, Sharpin and RIGI, Sharpin and MDA5, Sharpin and Tak1, Sharpin and TBK1, Sharpin and IKKe, Sharpin and IRF3, Sharpin and IRF7, Sharpin and IRF1, Sharpin and TRAF3, Sharpin and a Caspase, Sharpin and FADD, Sharpin and TNFR1, Sharpin and TRAILR1, Sharpin and TRAILR2, Sharpin and FAS, Sharpin and Bax, Sharpin and Bak, Sharpin and Bim, Sharpin and Bid, Sharpin and Noxa, Sharpin and Puma, Sharpin and TRIF, Sharpin and ZBP1, Sharpin and RIPK1, Sharpin and RIPK3, Sharpin and MLKL, Sharpin and Gasdermin A, Sharpin and Gasdermin B, Sharpin and Gasdermin C, Sharpin and Gasdermin D, Sharpin and Gasdermin E, IKKg and IKKa, IKKg and IKKb, IKKg and RelA, IKKg and MAVS, IKKg and RIGI, IKKg and MDA5, IKKg and Tak1, IKKg and TBK1, IKKg and IKKe, IKKg and IRF3, IKKg and IRF7, IKKg and IRF1, IKKg and TRAF3, IKKg and a Caspase, IKKg and FADD, IKKg and TNFR1, IKKg and TRAILR1, IKKg and TRAILR2, IKKg and FAS, IKKg and Bax, IKKg and Bak, IKKg and Bim, IKKg and Bid, IKKg and Noxa, IKKg and Puma, IKKg and TRIF, IKKg and ZBP1, IKKg and RIPK1, IKKg and RIPK3, IKKg and MLKL, IKKg and Gasdermin A, IKKg and Gasdermin B, IKKg and Gasdermin C, IKKg and Gasdermin D, IKKg and Gasdermin E, IKKa and IKKb, IKKa and RelA, IKKa and MAVS, IKKa and RIGI, IKKa and MDA5, IKKa and Tak1, IKKa and TBK1, IKKa and IKKe, IKKa and IRF3, IKKa and IRF7, IKKa and IRF1, IKKa and TRAF3, IKKa and a Caspase, IKKa and FADD, IKKa and TNFR1, IKKa and TRAILR1, IKKa and TRAILR2, IKKa and FAS, IKKa and Bax, IKKa and Bak, IKKa and Bim, IKKa and Bid, IKKa and Noxa, IKKa and Puma, IKKa and TRIF, IKKa and ZBP1, IKKa and RIPK1, IKKa and RIPK3, IKKa and MLKL, IKKa and Gasdermin A, IKKa and Gasdermin B, IKKa and Gasdermin C, IKKa and Gasdermin D, IKKa and Gasdermin E, IKKb and RelA, IKKb and MAVS, IKKb and RIGI, IKKb and MDA5, IKKb and Tak1, IKKb and TBK1, IKKb and IKKe, IKKb and IRF3, IKKb and IRF7, IKKb and IRF1, IKKb and TRAF3, IKKb and a Caspase, IKKb and FADD, IKKb and TNFR1, IKKb and TRAILR1, IKKb and TRAILR2, IKKb and FAS, IKKb and Bax, IKKb and Bak, IKKb and Bim, IKKb and Bid, IKKb and Noxa, IKKb and Puma, IKKb and TRIF, IKKb and ZBP1, IKKb and RIPK1, IKKb and RIPK3, IKKb and MLKL, IKKb and Gasdermin A, IKKb and Gasdermin B, IKKb and Gasdermin C, IKKb and Gasdermin D, IKKb and Gasdermin E, IKKb and RelA, IKKb and MAVS, IKKb and RIGI, IKKb and MDA5, IKKb and Tak1, IKKb and TBK1, IKKb and IKKe, IKKb and IRF3, IKKb and IRF7, IKKb and IRF1, IKKb and TRAF3, IKKb and a Caspase, IKKb and FADD, IKKb and TNFR1, IKKb and TRAILR1, IKKb and TRAILR2, IKKb and FAS, IKKb and Bax, IKKb and Bak, IKKb and Bim, IKKb and Bid, IKKb and Noxa, IKKb and Puma, IKKb and TRIF, IKKb and ZBP1, IKKb and RIPK1, IKKb and RIPK3, IKKb and MLKL, IKKb and Gasdermin A, IKKb and Gasdermin B, IKKb and Gasdermin C, IKKb and Gasdermin D, IKKb and Gasdermin E, RelA and MAVS, RelA and RIGI, RelA and MDA5, RelA and Tak1, RelA and TBK1, RelA and IKKe, RelA and IRF3, RelA and IRF7, RelA and IRF1, RelA and TRAF3, RelA and a Caspase, RelA and FADD, RelA and TNFR1, RelA and TRAILR1, RelA and TRAILR2, RelA and FAS, RelA and Bax, RelA and Bak, RelA and Bim, RelA and Bid, RelA and Noxa, RelA and Puma, RelA and TRIF, RelA and ZBP1, RelA and RIPK1, RelA and RIPK3, RelA and MLKL, RelA and Gasdermin A, RelA and Gasdermin B, RelA and Gasdermin C, RelA and Gasdermin D, RelA and Gasdermin E, MAVS and RIGI, MAVS and MDA5, MAVS and Tak1, MAVS and TBK1, MAVS and IKKe, MAVS and IRF3, MAVS and IRF7, MAVS and IRF1, MAVS and TRAF3, MAVS and a Caspase, MAVS and FADD, MAVS and TNFR1, MAVS and TRAILR1, MAVS and TRAILR2, MAVS and FAS, MAVS and Bax, MAVS and Bak, MAVS and Bim, MAVS and Bid, MAVS and Noxa, MAVS and Puma, MAVS and TRIF, MAVS and ZBP1, MAVS and RIPK1, MAVS and RIPK3, MAVS and MLKL, MAVS and Gasdermin A, MAVS and Gasdermin B, MAVS and Gasdermin C, MAVS and Gasdermin D, MAVS and Gasdermin E, RIGI and MDA5, RIGI and Tak1, RIGI and TBK1, RIGI and IKKe, RIGI and IRF3, RIGI and IRF7, RIGI and IRF1, RIGI and TRAF3, RIGI and a Caspase, RIGI and FADD, RIGI and TNFR1, RIGI and TRAILR1, RIGI and TRAILR2, RIGI and FAS, RIGI and Bax, RIGI and Bak, RIGI and Bim, RIGI and Bid, RIGI and Noxa, RIGI and Puma, RIGI and TRIF, RIGI and ZBP1, RIGI and RIPK1, RIGI and RIPK3, RIGI and MLKL, RIGI and Gasdermin A, RIGI and Gasdermin B, RIGI and Gasdermin C, RIGI and Gasdermin D, RIGI and Gasdermin E, MDA5 and Tak1, MDA5 and TBK1, MDA5 and IKKe, MDA5 and IRF3, MDA5 and IRF7, MDA5 and IRF1, MDA5 and TRAF3, MDA5 and a Caspase, MDA5 and FADD, MDA5 and TNFR1, MDA5 and TRAILR1, MDA5 and TRAILR2, MDA5 and FAS, MDA5 and Bax, MDA5 and Bak, MDA5 and Bim, MDA5 and Bid, MDA5 and Noxa, MDA5 and Puma, MDA5 and TRIF, MDA5 and ZBP1, MDA5 and RIPK1, MDA5 and RIPK3, MDA5 and MLKL, MDA5 and Gasdermin A, MDA5 and Gasdermin B, MDA5 and Gasdermin C, MDA5 and Gasdermin D, MDA5 and Gasdermin E, Tak1 and TBK1, Tak1 and IKKe, Tak1 and IRF3, Tak1 and IRF7, Tak1 and IRF1, Tak1 and TRAF3, Tak1 and a Caspase, Tak1 and FADD, Tak1 and TNFR1, Tak1 and TRAILR1, Tak1 and TRAILR2, Tak1 and FAS, Tak1 and Bax, Tak1 and Bak, Tak1 and Bim, Tak1 and Bid, Tak1 and Noxa, Tak1 and Puma, Tak1 and TRIF, Tak1 and ZBP1, Tak1 and RIPK1, Tak1 and RIPK3, Tak1 and MLKL, Tak1 and Gasdermin A, Tak1 and Gasdermin B, Tak1 and Gasdermin C, Tak1 and Gasdermin D, Tak1 and Gasdermin E, TB and IKKe, TB and IRF3, TB and IRF7, TB and IRF1, TB and TRAF3, TB and a Caspase, TBK1 and FADD, TBK1 and TNFR1, TBK1 and TRAILR1, TBK1 and TRAILR2, TBK1 and FAS, TBK1 and Bax, TBK1 and Bak, TBK1 and Bim, TBK1 and Bid, TBK1 and Noxa, TBK1 and Puma, TBK1 and TRIF, TBK1 and ZBP1, TBK1 and RIPK1, TBK1 and RIPK3, TBK1 and MLKL, TBK1 and Gasdermin A, TBK1 and Gasdermin B, TBK1 and Gasdermin C, TBK1 and Gasdermin D, TBK1 and Gasdermin E, IKKe and IRF3, IKKe and IRF7, IKKe and IRF1, IKKe and TRAF3, IKKe and a Caspase, IKKe and FADD, IKKe and TNFR1, IKKe and TRAILR1, IKKe and TRAILR2, IKKe and FAS, IKKe and Bax, IKKe and Bak, IKKe and Bim, IKKe and Bid, IKKe and Noxa, IKKe and Puma, IKKe and TRIF, IKKe and ZBP1, IKKe and RIPK1, IKKe and RIPK3, IKKe and MLKL, IKKe and Gasdermin A, IKKe and Gasdermin B, IKKe and Gasdermin C, IKKe and Gasdermin D, IKKe and Gasdermin E, IRF3 and IRF7, IRF3 and IRF1, IRF3 and TRAF3, IRF3 and a Caspase, IRF3 and FADD, IRF3 and TNFR1, IRF3 and TRAILR1, IRF3 and TRAILR2, IRF3 and FAS, IRF3 and Bax, IRF3 and Bak, IRF3 and Bim, IRF3 and Bid, IRF3 and Noxa, IRF3 and Puma, IRF3 and TRIF, IRF3 and ZBP1, IRF3 and RIPK1, IRF3 and RIPK3, IRF3 and MLKL, IRF3 and Gasdermin A, IRF3 and Gasdermin B, IRF3 and Gasdermin C, IRF3 and Gasdermin D, IRF3 and Gasdermin E, IRF7 and IRF1, IRF7 and TRAF3, IRF7 and a Caspase, IRF7 and FADD, IRF7 and TNFR1, IRF7 and TRAILR1, IRF7 and TRAILR2, IRF7 and FAS, IRF7 and Bax, IRF7 and Bak, IRF7 and Bim, IRF7 and Bid, IRF7 and Noxa, IRF7 and Puma, IRF7 and TRIF, IRF7 and ZBP1, IRF7 and RIPK1, IRF7 and RIPK3, IRF7 and MLKL, IRF7 and Gasdermin A, IRF7 and Gasdermin B, IRF7 and Gasdermin C, IRF7 and Gasdermin D, IRF7 and Gasdermin E, IRF1 and TRAF3, IRF1 and a Caspase, IRF1 and FADD, IRF1 and TNFR1, IRF1 and TRAILR1, IRF1 and TRAILR2, IRF1 and FAS, IRF1 and Bax, IRF1 and Bak, IRF1 and Bim, IRF1 and Bid, IRF1 and Noxa, IRF1 and Puma, IRF1 and TRIF, IRF1 and ZBP1, IRF1 and RIPK1, IRF1 and RIPK3, IRF1 and MLKL, IRF1 and Gasdermin A, IRF1 and Gasdermin B, IRF1 and Gasdermin C, IRF1 and Gasdermin D, IRF1 and Gasdermin E, TRAF3 and a Caspase, TRAF3 and FADD, TRAF3 and TNFR1, TRAF3 and TRAILR1, TRAF3 and TRAILR2, TRAF3 and FAS, TRAF3 and Bax, TRAF3 and Bak, TRAF3 and Bim, TRAF3 and Bid, TRAF3 and Noxa, TRAF3 and Puma, TRAF3 and TRIF, TRAF3 and ZBP1, TRAF3 and RIPK1, TRAF3 and RIPK3, TRAF3 and MLKL, TRAF3 and Gasdermin A, TRAF3 and Gasdermin B, TRAF3 and Gasdermin C, TRAF3 and Gasdermin D, TRAF3 and Gasdermin E, a Caspase and FADD, a Caspase and TNFR1, a Caspase and TRAILR1, a Caspase and TRAILR2, a Caspase and FAS, a Caspase and Bax, a Caspase and Bak, a Caspase and Bim, a Caspase and Bid, a Caspase and Noxa, a Caspase and Puma, a Caspase and TRIF, a Caspase and ZBP1, a Caspase and RIPK1, a Caspase and RIPK3, a Caspase and MLKL, a Caspase and Gasdermin A, a Caspase and Gasdermin B, a Caspase and Gasdermin C, a Caspase and Gasdermin D, a Caspase and Gasdermin E, FADD and TNFR1, FADD and TRAILR1, FADD and TRAILR2, FADD and FAS, FADD and Bax, FADD and Bak, FADD and Bim, FADD and Bid, FADD and Noxa, FADD and Puma, FADD and TRIF, FADD and ZBP1, FADD and RIPK1, FADD and RIPK3, FADD and MLKL, FADD and Gasdermin A, FADD and Gasdermin B, FADD and Gasdermin C, FADD and Gasdermin D, FADD and Gasdermin E, TNFR1 and TRAILR1, TNFR1 and TRAILR2, TNFR1 and FAS, TNFR1 and Bax, TNFR1 and Bak, TNFR1 and Bim, TNFR1 and Bid, TNFR1 and Noxa, TNFR1 and Puma, TNFR1 and TRIF, TNFR1 and ZBP1, TNFR1 and RIPK1, TNFR1 and RIPK3, TNFR1 and MLKL, TNFR1 and Gasdermin A, TNFR1 and Gasdermin B, TNFR1 and Gasdermin C, TNFR1 and Gasdermin D, TNFR1 and Gasdermin E, TRAILR1 and TRAILR2, TRAILR1 and FAS, TRAILR1 and Bax, TRAILR1 and Bak, TRAILR1 and Bim, TRAILR1 and Bid, TRAILR1 and Noxa, TRAILR1 and Puma, TRAILR1 and TRIF, TRAILR1 and ZBP1, TRAILR1 and RIPK1, TRAILR1 and RIPK3, TRAILR1 and MLKL, TRAILR1 and Gasdermin A, TRAILR1 and Gasdermin B, TRAILR1 and Gasdermin C, TRAILR1 and Gasdermin D, TRAILR1 and Gasdermin E, TRAILR2 and FAS, TRAILR2 and Bax, TRAILR2 and Bak, TRAILR2 and Bim, TRAILR2 and Bid, TRAILR2 and Noxa, TRAILR2 and Puma, TRAILR2 and TRIF, TRAILR2 and ZBP1, TRAILR2 and RIPK1, TRAILR2 and RIPK3, TRAILR2 and MLKL, TRAILR2 and Gasdermin A, TRAILR2 and Gasdermin B, TRAILR2 and Gasdermin C, TRAILR2 and Gasdermin D, TRAILR2 and Gasdermin E, FAS and Bax, F A S and Bak, F A S and Bim, F A S and Bid, F A S and Noxa, F A S and Puma, FAS and TRIF, FAS and ZBP1, FAS and RIPK1, FAS and RIPK3, FAS and MLKL, FAS and Gasdermin A, FAS and Gasdermin B, FAS and Gasdermin C, FAS and Gasdermin D, FAS and Gasdermin E, Bax and Bak, Bax and Bim, Bax and Bid, Bax and Noxa, Bax and Puma, Bax and TRIF, Bax and ZBP1, Bax and RIPK1, Bax and RIPK3, Bax and MLKL, Bax and Gasdermin A, Bax and Gasdermin B, Bax and Gasdermin C, Bax and Gasdermin D, Bax and Gasdermin E, Bak and Bim, Bak and Bid, Bak and Noxa, Bak and Puma, Bak and TRIF, Bak and ZBP1, Bak and RIPK1, Bak and RIPK3, Bak and MLKL, Bak and Gasdermin A, Bak and Gasdermin B, Bak and Gasdermin C, Bak and Gasdermin D, Bak and Gasdermin E, Bim and Bid, Bim and Noxa, Bim and Puma, Bim and TRIF, Bim and ZBP1, Bim and RIPK1, Bim and RIPK3, Bim and MLKL, Bim and Gasdermin A, Bim and Gasdermin B, Bim and Gasdermin C, Bim and Gasdermin D, Bim and Gasdermin E, Bid and Noxa, Bid and Puma, Bid and TRIF, Bid and ZBP1, Bid and RIPK1, Bid and RIPK3, Bid and MLKL, Bid and Gasdermin A, Bid and Gasdermin B, Bid and Gasdermin C, Bid and Gasdermin D, Bid and Gasdermin E, Noxa and Puma, Noxa and TRIF, Noxa and ZBP1, Noxa and RIPK1, Noxa and RIPK3, Noxa and MLKL, Noxa and Gasdermin A, Noxa and Gasdermin B, Noxa and Gasdermin C, Noxa and Gasdermin D, Noxa and Gasdermin E, Puma and TRIF, Puma and ZBP1, Puma and RIPK1, Puma and RIPK3, Puma and MLKL, Puma and Gasdermin A, Puma and Gasdermin B, Puma and Gasdermin C, Puma and Gasdermin D, Puma and Gasdermin E, TRIF and ZBP1, TRIF and RIPK1, TRIF and RIPK3, TRIF and MLKL, TRIF and Gasdermin A, TRIF and Gasdermin B, TRIF and Gasdermin C, TRIF and Gasdermin D, TRIF and Gasdermin E, ZBP1 and RIPK1, ZBP1 and RIPK3, ZBP1 and MLKL, ZBP1 and Gasdermin A, ZBP1 and Gasdermin B, ZBP1 and Gasdermin C, ZBP1 and Gasdermin D, ZBP1 and Gasdermin E, RIPK1 and RIPK3, RIPK1 and MLKL, RIPK1 and Gasdermin A, RIPK1 and Gasdermin B, RIPK1 and Gasdermin C, RIPK1 and Gasdermin D, RIPK1 and Gasdermin E, RIPK3 and MLKL, RIPK3 and Gasdermin A, RIPK3 and Gasdermin B, RIPK3 and Gasdermin C, RIPK3 and Gasdermin D, RIPK3 and Gasdermin E, MLKL and Gasdermin A, MLKL and Gasdermin B, MLKL and Gasdermin C, MLKL and Gasdermin D, MLKL and Gasdermin E, Gasdermin A and Gasdermin B, Gasdermin A and Gasdermin C, Gasdermin A and Gasdermin D, Gasdermin A and Gasdermin E, Gasdermin B and Gasdermin C, Gasdermin B and Gasdermin D, Gasdermin B and Gasdermin E, Gasdermin C and Gasdermin D, Gasdermin C and Gasdermin E, Gasdermin D and Gasdermin E, TNFSF protein and TRADD, TNFSF protein and TRAF2, TNFSF protein and TRAF6, TNFSF protein and cIAP1, TNFSF protein and cIAP2, TNFSF protein and XIAP, TNFSF protein and NOD2, TNFSF protein and MyD88, TNFSF protein and TRAM, TNFSF protein and HOIL, TNFSF protein and HOIP, TNFSF protein and Sharpin, TNFSF protein and IKKg, TNFSF protein and IKKa, TNFSF protein and IKKb, TNFSF protein and RelA, TNFSF protein and MAVS, TNFSF protein and RIGI, TNFSF protein and MDA5, TNFSF protein and Tak1, TNFSF protein and TBK1, TNFSF protein and IKKe, TNFSF protein and IRF3, TNFSF protein and IRF7, TNFSF protein and IRF1, TNFSF protein and TRAF3, TNFSF protein and a Caspase, TNFSF protein and FADD, TNFSF protein and TNFR1, TNFSF protein and TRAILR1, TNFSF protein and TRAILR2, TNFSF protein and FAS, TNFSF protein and Bax, TNFSF protein and Bak, TNFSF protein and Bim, TNFSF protein and Bid, TNFSF protein and Noxa, TNFSF protein and Puma, TNFSF protein and TRIF, TNFSF protein and ZBP1, TNFSF protein and RIPK1, TNFSF protein and RIPK3, TNFSF protein and MLKL, TNFSF protein and Gasdermin A, TNFSF protein and Gasdermin B, TNFSF protein and Gasdermin C, TNFSF protein and Gasdermin D, TNFSF protein and Gasdermin E, and variants thereof, and functional fragments thereof.

In a particular embodiment, at least one of the thanotransmission polypeptides is TRIF or a functional fragment or variant thereof.

In a particular embodiment, at least one of the thanotransmission polypeptides is RIPK3 or a functional fragment or variant thereof.

In a particular embodiment, at least one of the thanotransmission polypeptides encoded by the one or more thanotransmission polynucleotides comprises TRIF or a functional fragment thereof, and at least one of the thanotransmission polypeptides encoded by the one or more polynucleotides comprises RIPK3 or a functional fragment thereof.

In a particular embodiment, at least one of the thanotransmission polypeptides is MAVS or a functional fragment or variant thereof, and at least one of the thanotransmission polypeptides is RIPK3 or a functional fragment or variant thereof.

In a particular embodiment, at least one of the thanotransmission polypeptides is MAVS or a functional fragment or variant thereof, and at least one of the thanotransmission polypeptides is MLKL or a functional fragment or variant thereof.

In some embodiments, the functional fragment of Bid is truncated Bid (tBID). TNFR1/Fas engagement results in the cleavage of cytosolic Bid to truncated tBID, which translocates to mitochondria. The tBID polypeptide functions as a membrane-targeted death ligand. Bak-deficient mitochondria and blocking antibodies reveal tBID binds to its mitochondrial partner BAK to release cytochrome c. Activated tBID results in an allosteric activation of BAK, inducing its intramembranous oligomerization into a proposed pore for cytochrome c efflux, integrating the pathway from death receptors to cell demise. See Wei et al., 2000, Genes & Dev. 14: 2060-2071.

In a particular embodiment, at least one of the thanotransmission polypeptides is MAVS or a functional fragment or variant thereof, and at least one of the thanotransmission polypeptides is tBID or a functional fragment or variant thereof.

In some embodiments, the virus engineered to comprise one or more polynucleotides that promote thanotransmission does not comprise a polynucleotide encoding TRIF.

Additional polynucleotides to be included in the engineered viruses are described below.

### Caspase Inhibitors

The engineered virus may further comprise one or more polynucleotides that inhibit caspase activity in a target cell. In some embodiments, the polynucleotide that inhibits caspase activity in a target cell reduces expression or activity of one or more caspases that is endogenous to the target cell. Polynucleotides that reduce expression of a caspase may include, but are not limited to, antisense DNA molecules, antisense RNA molecules, double stranded RNA, siRNA, or a Clustered Regularly Interspaced Short Palindromic Repeats (CRISPR)-CRISPR associated (Cas) (CRISPR-Cas) system guide RNA.

In some embodiments, the polynucleotide that inhibits caspase activity in a target cell encodes a polypeptide that inhibits caspase activity. In some embodiments the polypeptide that inhibits caspase activity is a viral protein or a variant or functional fragment thereof. Exemplary viral protein caspase inhibitors are provided in Table 6 below. In some embodiments, the polypeptide that inhibits caspase activity is a human protein or a variant or functional fragment thereof. In some embodiments, the polypeptide that inhibits caspase activity inhibits one or more caspases selected from the group consisting of caspase 1, caspase 2, caspase 3, caspase 4, caspase 5, caspase 6, caspase 7, caspase 8, caspase 9 and caspase 10. In a particular embodiment, the polypeptide that inhibits caspase activity inhibits caspase 8. In a particular embodiment, the polypeptide that inhibits caspase activity inhibits caspase 10. In a particular embodiment, the polypeptide that inhibits caspase activity inhibits caspase 8 and caspase 10.

TABLE 6
Exemplary viral protein caspase inhibitors. (Adapted from Mocarski et al.,
2011, Nat Rev Immunol Dec 23; 12(2): 79-88. doi: 10.1038/nri3131, which
is incorporated by reference herein in its entirety.) (Abbreviations used
include: BHV-4, bovine herpesvirus 4; CMV, cytomegalovirus; DAI, DNA-dependent
activator of interferon regulatory factors; EHV-1, equine herpesvirus 1;
FADD, FAS-associated death domain protein; HPV-16, human papillomavirus
16; HSV, herpes simplex virus; KSHV, Kaposi's sarcoma-associated herpesvirus;
MCMV, murine cytomegalovirus; MCV, molluscum contagiosum virus; RHIM, RIP
homotypic interaction motif; RIP, receptor-interacting protein; TRIF, TIR
domain-containing adaptor protein inducing IFNβ; vICA, viral inhibitor
of caspase 8 activation; vIRA, viral inhibitor of RIP activation.)

| | | | | | Gene ID or |
|---|---|---|---|---|---|
| Type of | | | Known | | accession |
| inhibitor | Inhibitor | Virus | targets | Mechanism | number |
| cFLIP | MC159 | MCV | Caspase 8 | Inhibits | 1487017 |
| homologue | | | FADD | oligomerization | |
| cFLIP | K13 | KSHV | Caspase 8 | Prevents | 4961494 |
| homologue | | | | activation | |
| cFLIP | E8 | EHV-1 | Caspase 8 | — | 1461076 |
| homologue | | | | | |
| Caspase 8 | vICA | CMV | Caspase 8 | Prevents | 3077442 |
| inhibitor | | | | activation | |
| Caspase 8 | BORFE2 | BHV-4 | Caspase 8 | — | 1684940 |
| inhibitor | | | | | |
| Caspase 8 | E3 14.7 | Adenovirus | Caspase 8 | Prevents | 1460862 |
| inhibitor | kDa | | | activation | |
| Caspase 8 | UL39 | HSV-1, | Caspase 8 | Prevents | 2703361 |
| inhibitor | | HSV-2 | | activation | 1487325 |
| Serpin | CrmA | Cowpox | Caspases 1, | Inhibits activity | 1486086 |
| | | virus | 4, 5, 8 and | | |
| | | | 10, | | |
| | | | granzyme | | |
| | | | B | | |
| Serpin | B13R | Vaccinia | Caspases | — | 3707572 |
| | | virus | | | |
| Serpin | Serp2 | Myxoma | Caspases | — | 932102 |
| | | virus | | | |
| Other | E6 | HPV-16 | Caspase 8, | Inhibits | 1489078 |
| | | | FADD | oligomerization, | |
| | | | | degrades | |
| Other | P35 | Baculovirus | Caspases | Inhibits activity | 1403968 |

In some embodiments, the polypeptide that inhibits caspase activity is selected from the group consisting of a Fas Associated Death Domain protein (FADD) dominant negative mutant (FADD-DN), viral inhibitor of caspase 8 activation (vICA), cellular FLICE (FADD-like IL-1β-converting enzyme)-inhibitory protein (cFLIP), a caspase 8 dominant negative mutant (Casp8-DN), cellular inhibitor of apoptosis protein-1 (cIAP1), cellular inhibitor of apoptosis protein-1 (cIAP2), X-Linked Inhibitor Of Apoptosis (XIAP), TGFβ-activated kinase 1 (Tak1), an IκB kinase (IKK), and functional fragments thereof.

In a particular embodiment, the polypeptide that inhibits caspase activity is FADD-DN. The Death Inducing Signaling Complex (DISC) recruits adaptor proteins including FADD and initiator caspases such as caspase 8. See Morgan et al., 2001, Cell Death & Differentiation volume 8, pages 696-705. Aggregation of caspase 8 in the DISC leads to the activation of a caspase cascade and apoptosis. FADD consists of two protein interaction domains: a death domain and a death effector domain. Because FADD is an essential component of the DISC, a dominant negative mutant (FADD-DN) that contains the death domain but no death effector domain has been widely used in studies of death receptor-induced apoptosis. FADD-DN functions as a dominant negative inhibitor because it binds to the receptor but cannot recruit caspase 8.

In a particular embodiment, the polypeptide that inhibits caspase activity is vICA. The vICA protein is a human cytomegalovirus (CMV) protein encoded by the UL36 gene. See Skaletskaya et al., PNAS Jul. 3, 2001 98 (14) 7829-7834, which is incorporated by reference herein in its entirety. The vICA protein inhibits Fas-mediated apoptosis by binding to the pro-domain of caspase-8 and preventing its activation.

In a particular embodiment, the polypeptide that inhibits caspase activity is cFLIP. The cFLIP protein is a master anti-apoptotic regulator and resistance factor that suppresses tumor necrosis factor-α (TNF-α), Fas-L, and TNF-related apoptosis-inducing ligand (TRAIL)-induced apoptosis. See Safa, 2012, Exp Oncol October; 34(3):176-84, which is incorporated by reference herein in its entirety. The cFLIP protein is expressed as long (cFLIP(L)), short (cFLIP(S)), and cFLIP(R) splice variants in human cells. The cFLIP protein binds to FADD and/or caspase-8 or - and TRAIL receptor 5 (DRS) in a ligand-dependent and -independent fashion and forms an apoptosis inhibitory complex (AIC). This interaction in turn prevents death-inducing signaling complex (DISC) formation and subsequent activation of the caspase cascade. c-FLIP(L) and c-FLIP(S) are also known to have multifunctional roles in various signaling pathways. In a particular embodiment, the cFLIP is cFLIP(L). In a particular embodiment, the cFLIP is cFLIP(S).

In some embodiments, at least one of the thanotransmission polypeptides is TRIF or a functional fragment or variant thereof, at least one of the thanotransmission polypeptides is RIPK3 or a functional fragment or variant thereof, and at least one of the thanotransmission polypeptides is FADD-DN or a functional fragment or variant thereof.

In some embodiments, at least one of the thanotransmission polypeptides is TRIF or a functional fragment or variant thereof, at least one of the thanotransmission polypeptides is RIPK3 or a functional fragment or variant thereof, and at least one of the thanotransmission polypeptides is vICA or a functional fragment or variant thereof.

In some embodiments, at least one of the thanotransmission polypeptides is TRIF or a functional fragment or variant thereof, at least one of the thanotransmission polypeptides is RIPK3 or a functional fragment or variant thereof, and at least one of the thanotransmission polypeptides is cFLIP or a functional fragment or variant thereof.

In some embodiments, at least one of the thanotransmission polypeptides is MAVS or a functional fragment or variant thereof, at least one of the thanotransmission polypeptides is RIPK3 or a functional fragment or variant thereof, and at least one of the thanotransmission polypeptides is FADD-DN or a functional fragment or variant thereof.

### Gasdermins

The gasdermins are a family of pore-forming effector proteins that cause membrane permeabilization and pyroptosis. The gasdermin proteins include Gasdermin A, Gasdermin B, Gasdermin C, Gasdermin D and Gasdermin E. Gasdermins contain a cytotoxic N-terminal domain and a C-terminal repressor domain connected by a flexible linker. Proteolytic cleavage between these two domains releases the intramolecular inhibition on the cytotoxic domain, allowing it to insert into cell membranes and form large oligomeric pores, which disrupts ion homeostasis and induces cell death. See Broz et al., 2020, Nature Reviews Immunology 20: 143-157, which is incorporated by reference herein in its entirety. For example, Gasdermin E (GSDME, also known as DFNAS) can be cleaved by caspase 3, thereby converting noninflammatory apoptosis to pyroptosis in GSDME-expressing cells. Similarly, caspases 1, 4 and 5 cleave and activate Gasdermin D.

In some embodiments, at least one of the thanotransmission polypeptidesencodes a gasdermin or a functional fragment or variant thereof. In some embodiments, the functional fragment of the gasdermin is an N-terminal domain of Gasdermin A, Gasdermin B, Gasdermin C, Gasdermin D or Gasdermin E.

In some embodiments, at least one of the thanotransmission polypeptides is TRIF or a functional fragment or variant thereof, at least one of the thanotransmission polypeptides is RIPK3 or a functional fragment or variant thereof, and at least one of the thanotransmission polypeptides is a gasdermin or a functional fragment or variant thereof.

In some embodiments, at least one of the thanotransmission polypeptides is TRIF or a functional fragment or variant thereof, at least one of the thanotransmission polypeptides is RIPK3 or a functional fragment or variant thereof, and at least one of the thanotransmission polypeptides is Gasdermin E or a functional fragment or variant thereof.

In some embodiments, at least one of the thanotransmission polypeptides is MAVS or a functional fragment or variant thereof, and at least one of the thanotransmission polypeptides is a Gasdermin D N-terminal domain or a functional fragment or variant thereof.

In some embodiments, at least one of the thanotransmission polypeptides is MAVS or a functional fragment or variant thereof, and at least one of the thanotransmission polypeptides is a Gasdermin E N-terminal domain or a functional fragment or variant thereof.

In some embodiments, at least one of the thanotransmission polypeptides is MAVS or a functional fragment or variant thereof, at least one of the thanotransmission polypeptides is tBID or a functional fragment or variant thereof, and at least one of the thanotransmission polypeptides is Gasdermin E or a functional fragment or variant thereof.

In addition to the one or more polynucleotides encoding polypeptides that promote thanotransmission, such as those provided above in Tables 2, 3, 4, 5, and 6 the engineered virus may further comprise one or more polynucleotides encoding an immune stimulatory protein. such as those described below.

### Immune Stimulatory Proteins

In addition to the one or more polynucleotides encoding polypeptides that promote thanotransmission, such as those provided above in Tables 2, 3, 4 and 5, the engineered viruses disclosed herein may further comprise one or more polynucleotides encoding an immune stimulatory protein. In one embodiment, the immune stimulatory protein is an antagonist of transforming growth factor beta (TGF-β), a colony-stimulating factor, a cytokine, an immune checkpoint modulator, an flt3 ligand or an antibody agonist of flt3.

The colony-stimulating factor may be a granulocyte-macrophage colony-stimulating factor (GM-CSF). In one embodiment, the polynucleotide encoding GM-CSF is inserted into the ICP34.5 gene locus.

The cytokine may be an interleukin. In one embodiment, the interleukin is selected from the group consisting of IL-1α, IL-1β, IL-2, IL-4, IL-12, IL-15, IL-18, IL-21, IL-24, IL-33, IL-36a, IL-36β and IL-36γ. Additional suitable cytokines include a type I interferon, interferon gamma, a type III interferon and TNFα.

In some embodiments, the immune checkpoint modulator is an antagonist of an inhibitory immune checkpoint protein. Examples of inhibitory immune checkpoint protein include, but are not limited to, ADORA2A, B7-H3, B7-H4, IDO, KIR, VISTA, PD-1, PD-L1, PD-L2, LAG3, Tim3, BTLA and CTLA4. In some embodiments, the immune checkpoint modulator is an agonist of a stimulatory immune checkpoint protein. Examples of stimulatory immune checkpoint proteins include, but are not limited to, CD27, CD28, CD40, CD122, OX40, GITR, ICOS and 4-1BB. In some embodiments, the agonist of the stimulatory immune checkpoint protein is selected from CD40 ligand (CD40L), ICOS ligand, GITR ligand, 4-1-BB ligand, 0X40 Ligand and a modified version of any thereof. In some embodiments, the agonist of the stimulatory immune checkpoint protein is an antibody agonist of a protein selected from CD40, ICOS, GITR, 4-1-BB and 0X40.

### Suicide Genes

In addition to the one or more polynucleotides encoding a polypeptide that promotes thanotransmission, such as those provided above in Table 2A, Table 2B. Table 3, Table 4, Table 5, or Table 6, the engineered viruses disclosed herein may further comprise a suicide gene. The term “suicide gene” refers to a gene encoding a protein (e.g., an enzyme) that converts a nontoxic precursor of a drug into a cytotoxic compound. In some embodiments, the suicide gene encodes a polypeptide selected from the group consisting of FK506 binding protein (FKBP)-FAS, FKBP-caspase-8, FKBP-caspase-9, a polypeptide having cytosine deaminase (CDase) activity, a polypeptide having thymidine kinase activity, a polypeptide having uracil phosphoribosyl transferase (UPRTase) activity, and a polypeptide having purine nucleoside phosphorylase activity.

In some embodiments, the polypeptide having CDase activity is FCY1, FCA1 or CodA.

In some embodiments, the polypeptide having UPRTase activity is FUR1 or a variant thereof, e.g. FUR1Δ105. FUR1Δ105 is an FUR1 gene lacking the first 105 nucleotides in the 5′ region of the coding region allowing the synthesis of a UPRTase from which the first 35 amino acid residues have been deleted at the N-terminus. FUR1Δ105 starts with the methionine at position 36 of the native protein.

The suicide gene may encode a chimeric protein, e.g. a chimeric protein having CDase and UPRTase activity. In some embodiments, the chimeric protein is selected from codA::upp, FCY1::FUR1, FCY1::FUR1Δ105 (FCU1) and FCU1-8 polypeptides.

The virus engineered to comprise one or more polynucleotides that promote thanotransmission may further comprise a polynucleotide encoding a matrix metalloproteinase, e.g. matrix metalloproteinase 9 (“MMP9), which degrades collagen type IV, a major component of the of the extracellular matrix (ECM) and basement membranes of glioblastomas (Mammato et al., Am. J. Pathol., 183(4): 1293-1305 (2013), doi: 10.1016/j.ajpath.2013.06.026. Epub 2013 Aug. 5). Expression of a matrix metalloproteinase by the engineered virus enhances infection of tumor cells by the virus due to lateral spread and enhancing tumor-killing activity. Polynucleotides encoding other genes that enhance lateral spread of the virus may also be used.

In some embodiments, the polynucleotide that promotes thanotransmission is a polynucleotide (e.g. a polynucleotide encoding an siRNA) that reduces expression or activity in the target cell of a polypeptide endogenous to the target cell that inhibits thanotransmission. Exemplary polypeptides endogenous to a target cell that may inhibit thanotransmission are provided in Table 7 below.

TABLE 7
Exemplary polypeptides that inhibit
thanotransmission in a target cell

| Polypeptide | Accession No. |
|---|---|
| FADD | NP_003815 |
| cIAP1 | NP_001157.1 |
| cIAP2 | NP_001156.1 |
| HOIL1 | Q9BYM8.2 |
| HOIP | Q96EP0.1 |
| Sharpin | NP_112236.3 |
| cFLIP | BAB32551.1 |
| A20 | AAA51550.1 |
| Tak1 | NP_003179.1 |
| IKKb | NP_001547.1 |
| IKKa | NP_001269.3 |
| IkBa | NP_065390.1 |
| P65 | AAI10831.1 |
| CYLD | CAB93533.1 |
| FSP1 | AK127353 |
| GPX4 | AC004151 |

Polynucleotides that reduce expression of genes that inhibit thanotransmission may include, but are not limited to, antisense DNA molecules, antisense RNA molecules, double stranded RNA, siRNA, or a Clustered Regularly Interspaced Short Palindromic Repeats (CRISPR)-CRISPR associated (Cas) (CRISPR-Cas) system guide RNA.

Expression of the one or more polynucleotides or polypeptides that promote thanotransmission from the virus upon infection of the target cell may alter a cell turnover pathway in the target cell. For example, expression of the one or more polynucleotides or polypeptides upon viral infection of the target cell may change the normal cell turnover pathway of the target cell to a cell turnover pathway that promotes thanotransmission, such as, e.g., programmed necrosis (e.g., necroptosis or pyroptosis), extrinsic apoptosis, or ferroptosis.

The mutations in viral genes described herein may be combined with the polynucleotides encoding proteins that promote thanotransmission and/or the polynucleotides that reduce expression of polypeptides that inhibit thanotransmission. In a particular embodiment, the virus is HSV1 comprising an inactivating mutation (e.g. a deletion) in the ICP34.5 and ICP47 genes, an inactivating mutation in the RHIM domain of ICP6, and polynucleotides encoding ZBP1, RIPK3 and MLKL. In a further particular embodiment, the virus is HSV1 comprising an inactivating mutation (e.g. a deletion) of ICP47, a replacement of ICP34.5 with a delta-Zα1 mutant form of the Vaccinia virus E3L gene, and polynucleotides encoding ZBP1, RIPK3 and MLKL. In a further particular embodiment, the virus is a Vaccinia virus comprising a mutation in the Zα1 domain of the E3L gene, and polynucleotides encoding ZBP1, RIPK3 and MLKL. In a further particular embodiment, the virus is an Ad5/F35 adenovirus comprising a 24 bp deletion in E1A and an 827 bp deletion in E1B.

The engineered viruses described herein may further comprise a heterologous promoter that is operably linked to a polynucleotide as described herein (e.g., a polynucleotide encoding a thanotransmision polypeptide) to drive expression of the polynucleotide. Suitable promoters include, but are not limited to, a CMV promoter (e.g., a mini-CMV promoter), an EF1α promoter (e.g., a mini-EF1α promoter), an SV40 promoter, a PGK1 promoter, a polyubiquitin C (UBC) gene promoter, a human beta actin promoter, and a CMV enhancer/chicken beta-actin/rabbit beta-globin (CAG) hybrid promoter. In some embodiments, the promoter is a cancer-specific promoter, e.g., a tumor-specific promoter. Suitable tumor-specific promoters include, but are not limited to, a human telomerase reverse transcriptase (hTERT) promoter and an E2F promoter. The hTERT promoter drives gene expression in cells (such as cancer cells) with increased expression of telomerase. The E2F promoter drives gene expression that is specific to cells with an altered Rb pathway.

### V. Target Cells for the Virus

The viruses engineered to comprise one or more polynucleotides that promote thanotransmission described herein may infect a range of different target cells to promote thanotransmission in the target cell. Types of target cells include, but are not limited to, cancer cells, immune cells, endothelial cells, fibroblasts, and cells infected with a pathogen.

Cells of any of the cancers described herein may be suitable as target cells for the engineered virus. In some embodiments, the target cell is a metastatic cancer cell.

In some embodiments, the target cell is an immune cell selected from mast cells, natural killer (NK) cells, monocytes, macrophages, dendritic cells, lymphocytes (e.g. B-cells and T cells) and any of the other immune cells described herein.

In some embodiments, the target cell is infected with a pathogen. Exemplary pathogens include a bacterium (e.g. a Gram-positive or Gram-negative bacterium), a fungus, a parasite, and a virus. Exemplary bacterial pathogens include E. coli, Klebsiella pneumoniae, Pseudomonas aeruginosa, Salmonella spp., Staphylococcus aureus, Streptococcus spp., or vancomycin-resistant Enterococcus). The fungal pathogen may be, for example, a mold, a yeast, or a higher fungus. The parasite may be, for example, a single-celled or multicellular parasite, including Giardia duodenalis, Cryptosporidium parvum, Cyclospora cayetanensis, and Toxoplasma gondiz. The virus may be a virus associated with AIDS, avian flu, chickenpox, cold sores, common cold, gastroenteritis, glandular fever, influenza, measles, mumps, pharyngitis, pneumonia, rubella, SARS, and lower or upper respiratory tract infection (e.g., respiratory syncytial virus). In some embodiments, the virus is hepatitis B virus or hepatitis C virus.

In some embodiments the target cell (e.g. a cancer cell) is deficient in a cell turnover pathway. For example, the target cell may have an inactivating mutation or copy number loss of a gene encoding a protein that contributes to the cell turnover pathway. In some embodiments, the target cell is deficient in an immune-stimulatory cell turnover pathway, e.g. programmed necrosis (e.g., necroptosis or pyroptosis), extrinsic apoptosis, ferroptosis, or combinations thereof. In some embodiments, the target cell has an inactivating mutation of one or more of a gene encoding receptor-interacting serine/threonine-protein kinase 3 (RIPK1), a gene encoding receptor-interacting serine/threonine-protein kinase 3 (RIPK3), a gene encoding Z-DNA-binding protein 1 (ZBP1), a gene encoding mixed lineage kinase domain like pseudokinase (MLKL), a gene encoding a gasdermin (e.g., Gasdermin D and/or Gasdermin E), and a gene encoding Toll/interleukin-1 receptor (TIR)-domain-containing adapter-inducing interferon-β (TRIF). In some embodiments, the target cell has reduced expression or activity of one or more of RIPK1, RIPK3, ZBP1, TRIF, a gasdermin (e.g., Gasdermin D and/or Gasdermin E), and MLKL. In some embodiments, the target cell does not express one or more of RIPK1, RIPK3, ZBP1, TRIF, a gasdermin (e.g., Gasdermin D and/or Gasdermin E), and MLKL. In some embodiments, the target cell has copy number loss of one or more of a gene encoding RIPK1, a gene encoding RIPK3, a gene encoding ZBP1, a gene encoding TRIF, a gene encoding a gasdermin (e.g., Gasdermin D and/or Gasdermin E), and a gene encoding MLKL.

In some embodiments, a subject is evaluated for any one or more of the target cell criteria described herein before, during, and/or after administration of a composition described herein.

### VI. Methods of Promoting Thanotransmission

The engineered viruses described herein may be used to promote thanotransmission by a target cell. In certain aspects, the disclosure relates to a method of promoting thanotransmission by a target cell, the method comprising contacting a target cell with a virus engineered to comprise one or more polynucleotides that promote thanotransmission by the target cell, wherein the target cell is contacted with the virus in an amount and for a time sufficient to promote thanotransmission by the target cell. For example, infection of the target cell with the engineered virus and expression of the one or more polynucleotides that promote thanotransmission induces the target cell to produce factors that are actively released by the target cell or become exposed during turnover (e.g. death) of the target cell. These factors signal a responding cell (e.g. an immune cell) to undergo a biological response (e.g. an increase in immune activity).

In some embodiments, the engineered virus is administered to a subject to promote thanotransmission by a target cell in the subject. For example, in certain aspects, the disclosure relates to a method of delivering one or more thanotransmission polynucleotides to a subject, the method comprising administering a pharmaceutical composition comprising an engineered virus as described herein to the subject. In certain aspects, the disclosure relates to a method of promoting thanotransmission in a subject, the method comprising administering a pharmaceutical composition comprising an engineered virus as described herein to the subject in an amount and for a time sufficient to promote thanotransmission.

A. Methods of Increasing Immune Activity

In one aspect, the engineered viruses described herein may be used to increase immune activity in a subject, for example, a subject who would benefit from increased immune activity. In certain aspects, the disclosure relates to a method of promoting an immune response in a subject in need thereof, the method comprising administering to the subject a virus engineered to comprise one or more polynucleotides that promote thanotransmission by the target cell, wherein the virus is administered to the subject in an amount and for a time sufficient to promote thanotransmission, thereby promoting an immune response in the subject. For example, factors produced by the target cell upon expression of the one or more polynucleotides that promote thanotransmission may induce an immuno-stimulatory response (e.g., a pro-inflammatory response) in a responding cell (e.g., an immune cell). In one embodiment, the immune response is an anti-cancer response.

According to the methods of the disclosure, immune activity may be modulated by interaction of the target cell with a broad range of immune cells, including, for example, any one or more of mast cells, Natural Killer (NK) cells, basophils, neutrophils, monocytes, macrophages, dendritic cells, eosinophils, lymphocytes (e.g. B-lymphocytes (B-cells)), and T-lymphocytes (T-cells)).

### Types of Immune Cells

Mast cells are a type of granulocyte containing granules rich in histamine and heparin, an anti-coagulant. When activated, a mast cell releases inflammatory compounds from the granules into the local microenvironment. Mast cells play a role in allergy, anaphylaxis, wound healing, angiogenesis, immune tolerance, defense against pathogens, and blood-brain barrier function.

Natural Killer (NK) cells are cytotoxic lymphocytes that lyse certain tumor and virus infected cells without any prior stimulation or immunization. NK cells are also potent producers of various cytokines, e.g. IFN-gamma (IFNγ), TNF-alpha (TNFα), GM-CSF and IL-3. Therefore, NK cells are also believed to function as regulatory cells in the immune system, influencing other cells and responses. In humans, NK cells are broadly defined as CD56+CD3-lymphocytes. The cytotoxic activity of NK cells is tightly controlled by a balance between the activating and inhibitory signals from receptors on the cell surface. A main group of receptors that inhibits NK cell activation are the inhibitory killer immunoglobulin-like receptors (KIRs). Upon recognition of self MHC class I molecules on the target cells, these receptors deliver an inhibitory signal that stops the activating signaling cascade, keeping cells with normal MHC class I expression from NK cell lysis. Activating receptors include the natural cytotoxicity receptors (NCR) and NKG2D that push the balance towards cytolytic action through engagement with different ligands on the target cell surface. Thus, NK cell recognition of target cells is tightly regulated by processes involving the integration of signals delivered from multiple activating and inhibitory receptors.

Monocytes are bone marrow-derived mononuclear phagocyte cells that circulate in the blood for few hours/days before being recruited into tissues. See Wacleche et al., 2018, Viruses (10)2: 65. The expression of various chemokine receptors and cell adhesion molecules at their surface allows them to exit the bone marrow into the blood and to be subsequently recruited from the blood into tissues. Monocytes belong to the innate arm of the immune system providing responses against viral, bacterial, fungal or parasitic infections. Their functions include the killing of pathogens via phagocytosis, the production of reactive oxygen species (ROS), nitric oxide (NO), myeloperoxidase and inflammatory cytokines. Under specific conditions, monocytes can stimulate or inhibit T-cell responses during cancer as well as infectious and autoimmune diseases. They are also involved in tissue repair and neovascularization.

Macrophages engulf and digest substances such as cellular debris, foreign substances, microbes and cancer cells in a process called phagocytosis. Besides phagocytosis, macrophages play a critical role in nonspecific defense (innate immunity) and also help initiate specific defense mechanisms (adaptive immunity) by recruiting other immune cells such as lymphocytes. For example, macrophages are important as antigen presenters to T cells. Beyond increasing inflammation and stimulating the immune system, macrophages also play an important anti-inflammatory role and can decrease immune reactions through the release of cytokines. Macrophages that encourage inflammation are called M1 macrophages, whereas those that decrease inflammation and encourage tissue repair are called M2 macrophages.

Dendritic cells (DCs) play a critical role in stimulating immune responses against pathogens and maintaining immune homeostasis to harmless antigens. DCs represent a heterogeneous group of specialized antigen-sensing and antigen-presenting cells (APCs) that are essential for the induction and regulation of immune responses. In the peripheral blood, human DCs are characterized as cells lacking the T-cell (CD3, CD4, CD8), the B-cell (CD19, CD20) and the monocyte markers (CD14, CD16) but highly expressing HLA-DR and other DC lineage markers (e.g., CD1a, CD1c). See Murphy et al., Janeway's Immunobiology. 8th ed. Garland Science; New York, NY, USA: 2012. 868p.

The term “lymphocyte” refers to a small white blood cell formed in lymphatic tissue throughout the body and in normal adults making up about 22-28% of the total number of leukocytes in the circulating blood that plays a large role in defending the body against disease. Individual lymphocytes are specialized in that they are committed to respond to a limited set of structurally related antigens through recombination of their genetic material (e.g. to create a T cell receptor and a B cell receptor). This commitment, which exists before the first contact of the immune system with a given antigen, is expressed by the presence of receptors specific for determinants (epitopes) on the antigen on the lymphocyte's surface membrane. Each lymphocyte possesses a unique population of receptors, all of which have identical combining sites. One set, or clone, of lymphocytes differs from another clone in the structure of the combining region of its receptors and thus differs in the epitopes that it can recognize. Lymphocytes differ from each other not only in the specificity of their receptors, but also in their functions. (Paul, W. E., “Chapter 1: The immune system: an introduction,” Fundamental Immunology, 4th Edition, Ed. Paul, W. E., Lippicott-Raven Publishers, Philadelphia, (1999), at p. 102).

Lymphocytes include B-lymphocytes (B-cells), which are precursors of antibody-secreting cells, and T-lymphocytes (T-cells).

B-Lymphocytes (B-Cells)

B-lymphocytes are derived from hematopoietic cells of the bone marrow. A mature B-cell can be activated with an antigen that expresses epitopes that are recognized by its cell surface. The activation process may be direct, dependent on cross-linkage of membrane Ig molecules by the antigen (cross-linkage-dependent B-cell activation), or indirect, via interaction with a helper T-cell, in a process referred to as cognate help. In many physiological situations, receptor cross-linkage stimuli and cognate help synergize to yield more vigorous B-cell responses (Paul, W. E., “Chapter 1: The immune system: an introduction,” Fundamental Immunology, 4th Edition, Ed. Paul, W. E., Lippicott-Raven Publishers, Philadelphia, (1999)).

Cross-linkage dependent B-cell activation requires that the antigen express multiple copies of the epitope complementary to the binding site of the cell surface receptors, because each B-cell expresses Ig molecules with identical variable regions. Such a requirement is fulfilled by other antigens with repetitive epitopes, such as capsular polysaccharides of microorganisms or viral envelope proteins. Cross-linkage-dependent B-cell activation is a major protective immune response mounted against these microbes (Paul, W. E., “Chapter 1: The immune system: an introduction”, Fundamental Immunology, 4th Edition, Ed. Paul, W. E., Lippicott-Raven Publishers, Philadelphia, (1999)).

Cognate help allows B-cells to mount responses against antigens that cannot cross-link receptors and, at the same time, provides costimulatory signals that rescue B cells from inactivation when they are stimulated by weak cross-linkage events. Cognate help is dependent on the binding of antigen by the B-cell's membrane immunoglobulin (Ig), the endocytosis of the antigen, and its fragmentation into peptides within the endosomal/lysosomal compartment of the cell. Some of the resultant peptides are loaded into a groove in a specialized set of cell surface proteins known as class II major histocompatibility complex (MHC) molecules. The resultant class II/peptide complexes are expressed on the cell surface and act as ligands for the antigen-specific receptors of a set of T-cells designated as CD4⁺ T-cells. The CD4⁺ T-cells bear receptors on their surface specific for the B-cell's class II/peptide complex. B-cell activation depends not only on the binding of the T cell through its T cell receptor (TCR), but this interaction also allows an activation ligand on the T-cell (CD40 ligand) to bind to its receptor on the B-cell (CD40) signaling B-cell activation. In addition, T helper cells secrete several cytokines that regulate the growth and differentiation of the stimulated B-cell by binding to cytokine receptors on the B cell (Paul, W. E., “Chapter 1: The immune system: an introduction, “Fundamental Immunology, 4th Edition, Ed. Paul, W. E., Lippicott-Raven Publishers, Philadelphia, (1999)).

During cognate help for antibody production, the CD40 ligand is transiently expressed on activated CD4⁺ T helper cells, and it binds to CD40 on the antigen-specific B cells, thereby transducing a second costimulatory signal. The latter signal is essential for B cell growth and differentiation and for the generation of memory B cells by preventing apoptosis of germinal center B cells that have encountered antigen. Hyperexpression of the CD40 ligand in both B and T cells is implicated in pathogenic autoantibody production in human SLE patients (Desai-Mehta, A. et al., “Hyperexpression of CD40 ligand by B and T cells in human lupus and its role in pathogenic autoantibody production,” J. Clin. Invest. Vol. 97(9), 2063-2073, (1996)).

T-Lymphocytes (T-Cells)

T-lymphocytes derived from precursors in hematopoietic tissue, undergo differentiation in the thymus, and are then seeded to peripheral lymphoid tissue and to the recirculating pool of lymphocytes. T-lymphocytes or T cells mediate a wide range of immunologic functions. These include the capacity to help B cells develop into antibody-producing cells, the capacity to increase the microbicidal action of monocytes/macrophages, the inhibition of certain types of immune responses, direct killing of target cells, and mobilization of the inflammatory response. These effects depend on T cell expression of specific cell surface molecules and the secretion of cytokines (Paul, W. E., “Chapter 1: The immune system: an introduction”, Fundamental Immunology, 4th Edition, Ed. Paul, W. E., Lippicott-Raven Publishers, Philadelphia, (1999)).

T cells differ from B cells in their mechanism of antigen recognition. Immunoglobulin, the B cell's receptor, binds to individual epitopes on soluble molecules or on particulate surfaces. B-cell receptors see epitopes expressed on the surface of native molecules. While antibody and B-cell receptors evolved to bind to and to protect against microorganisms in extracellular fluids, T cells recognize antigens on the surface of other cells and mediate their functions by interacting with, and altering, the behavior of these antigen-presenting cells (APCs). There are three main types of APCs in peripheral lymphoid organs that can activate T cells: dendritic cells, macrophages and B cells. The most potent of these are the dendritic cells, whose only function is to present foreign antigens to T cells. Immature dendritic cells are located in tissues throughout the body, including the skin, gut, and respiratory tract. When they encounter invading microbes at these sites, they endocytose the pathogens and their products, and carry them via the lymph to local lymph nodes or gut associated lymphoid organs. The encounter with a pathogen induces the dendritic cell to mature from an antigen-capturing cell to an APC that can activate T cells. APCs display three types of protein molecules on their surface that have a role in activating a T cell to become an effector cell: (1) MHC proteins, which present foreign antigen to the T cell receptor; (2) costimulatory proteins which bind to complementary receptors on the T cell surface; and (3) cell-cell adhesion molecules, which enable a T cell to bind to the APC for long enough to become activated (“Chapter 24: The adaptive immune system,” Molecular Biology of the Cell, Alberts, B. et al., Garland Science, NY, (2002)).

T-cells are subdivided into two distinct classes based on the cell surface receptors they express. The majority of T cells express T cell receptors (TCR) consisting of α and β-chains. A small group of T cells express receptors made of γ and δ chains. Among the α/β T cells are two sub-lineages: those that express the coreceptor molecule CD4 (CD4⁺ T cells); and those that express CD8 (CD8⁺ T cells). These cells differ in how they recognize antigen and in their effector and regulatory functions.

CD4⁺ T cells are the major regulatory cells of the immune system. Their regulatory function depends both on the expression of their cell-surface molecules, such as CD40 ligand whose expression is induced when the T cells are activated, and the wide array of cytokines they secrete when activated.

T cells also mediate important effector functions, some of which are determined by the patterns of cytokines they secrete. The cytokines can be directly toxic to target cells and can mobilize potent inflammatory mechanisms.

In addition, T cells, particularly CD8⁺ T cells, can develop into cytotoxic T-lymphocytes (CTLs) capable of efficiently lysing target cells that express antigens recognized by the CTLs (Paul, W. E., “Chapter 1: The immune system: an introduction,” Fundamental Immunology, 4th Edition, Ed. Paul, W. E., Lippicott-Raven Publishers, Philadelphia, (1999)).

T cell receptors (TCRs) recognize a complex consisting of a peptide derived by proteolysis of the antigen bound to a specialized groove of a class II or class I MHC protein. CD4⁺ T cells recognize only peptide/class II complexes while CD8⁺ T cells recognize peptide/class I complexes (Paul, W. E., “Chapter 1: The immune system: an introduction,” Fundamental Immunology, 4th Edition, Ed. Paul, W. E., Lippicott-Raven Publishers, Philadelphia, (1999)).

The TCR's ligand (i.e., the peptide/MHC protein complex) is created within APCs. In general, class II MHC molecules bind peptides derived from proteins that have been taken up by the APC through an endocytic process. These peptide-loaded class II molecules are then expressed on the surface of the cell, where they are available to be bound by CD4⁺ T cells with TCRs capable of recognizing the expressed cell surface complex. Thus, CD4⁺ T cells are specialized to react with antigens derived from extracellular sources (Paul, W. E., “Chapter 1: The immune system: an introduction,” Fundamental Immunology, 4th Edition, Ed. Paul, W. E., Lippicott-Raven Publishers, Philadelphia, (1999)).

In contrast, class I MHC molecules are mainly loaded with peptides derived from internally synthesized proteins, such as viral proteins. These peptides are produced from cytosolic proteins by proteolysis by the proteosome and are translocated into the rough endoplasmic reticulum. Such peptides, generally composed of nine amino acids in length, are bound into the class I MHC molecules and are brought to the cell surface, where they can be recognized by CD8⁺ T cells expressing appropriate receptors. This gives the T cell system, particularly CD8⁺ T cells, the ability to detect cells expressing proteins that are different from, or produced in much larger amounts than, those of cells of the remainder of the organism (e.g., viral antigens) or mutant antigens (such as active oncogene products), even if these proteins in their intact form are neither expressed on the cell surface nor secreted (Paul, W. E., “Chapter 1: The immune system: an introduction,” Fundamental Immunology, 4th Edition, Ed. Paul, W. E., Lippicott-Raven Publishers, Philadelphia, (1999)).

T cells can also be classified based on their function as helper T cells; T cells involved in inducing cellular immunity; suppressor T cells; and cytotoxic T cells.

Helper T Cells

Helper T cells are T cells that stimulate B cells to make antibody responses to proteins and other T cell-dependent antigens. T cell-dependent antigens are immunogens in which individual epitopes appear only once or a limited number of times such that they are unable to cross-link the membrane immunoglobulin (Ig) of B cells or do so inefficiently. B cells bind the antigen through their membrane Ig, and the complex undergoes endocytosis. Within the endosomal and lysosomal compartments, the antigen is fragmented into peptides by proteolytic enzymes, and one or more of the generated peptides are loaded into class II MHC molecules, which traffic through this vesicular compartment. The resulting peptide/class II MHC complex is then exported to the B-cell surface membrane. T cells with receptors specific for the peptide/class II molecular complex recognize this complex on the B-cell surface. (Paul, W. E., “Chapter 1: The immune system: an introduction,” Fundamental Immunology, 4th Edition, Ed. Paul, W. E., Lippicott-Raven Publishers, Philadelphia (1999)).

B-cell activation depends both on the binding of the T cell through its TCR and on the interaction of the T-cell CD40 ligand (CD40L) with CD40 on the B cell. T cells do not constitutively express CD40L. Rather, CD40L expression is induced as a result of an interaction with an APC that expresses both a cognate antigen recognized by the TCR of the T cell and CD80 or CD86. CD80/CD86 is generally expressed by activated, but not resting, B cells so that the helper interaction involving an activated B cell and a T cell can lead to efficient antibody production. In many cases, however, the initial induction of CD40L on T cells is dependent on their recognition of antigen on the surface of APCs that constitutively express CD80/86, such as dendritic cells. Such activated helper T cells can then efficiently interact with and help B cells. Cross-linkage of membrane Ig on the B cell, even if inefficient, may synergize with the CD40L/CD40 interaction to yield vigorous B-cell activation. The subsequent events in the B-cell response, including proliferation, Ig secretion, and class switching of the Ig class being expressed, either depend or are enhanced by the actions of T cell-derived cytokines (Paul, W. E., “Chapter 1: The immune system: an introduction,” Fundamental Immunology, 4th Edition, Ed. Paul, W. E., Lippicott-Raven Publishers, Philadelphia, (1999)).

CD4⁺ T cells tend to differentiate into cells that principally secrete the cytokines IL-4, IL-5, IL-6, and IL-10 (T_{H}2 cells) or into cells that mainly produce IL-2, IFN-γ, and lymphotoxin (T_{H}1 cells). The T_{H}2 cells are very effective in helping B-cells develop into antibody-producing cells, whereas the T_{H}1 cells are effective inducers of cellular immune responses, involving enhancement of microbicidal activity of monocytes and macrophages, and consequent increased efficiency in lysing microorganisms in intracellular vesicular compartments. Although CD4⁺ T cells with the phenotype of T_{H}2 cells (i.e., IL-4, IL-5, IL-6 and IL-10) are efficient helper cells, T_{H}1 cells also have the capacity to be helpers (Paul, W. E., “Chapter 1: The immune system: an introduction, “Fundamental Immunology, 4th Edition, Ed. Paul, W. E., Lippicott-Raven Publishers, Philadelphia, (1999)).

T Cell Involvement in Cellular Immunity Induction

T cells also may act to enhance the capacity of monocytes and macrophages to destroy intracellular microorganisms. In particular, interferon-gamma (IFN-γ) produced by helper T cells enhances several mechanisms through which mononuclear phagocytes destroy intracellular bacteria and parasitism including the generation of nitric oxide and induction of tumor necrosis factor (TNF) production. T_{H1} cells are effective in enhancing the microbicidal action, because they produce IFN-γ. In contrast, two of the major cytokines produced by T_{H2} cells, IL-4 and IL-10, block these activities (Paul, W. E., “Chapter 1: The immune system: an introduction,” Fundamental Immunology, 4th Edition, Ed. Paul, W. E., Lippicott-Raven Publishers, Philadelphia, (1999)).

Regulatory T (Treg) Cells

Immune homeostasis is maintained by a controlled balance between initiation and downregulation of the immune response. The mechanisms of both apoptosis and T cell anergy (a tolerance mechanism in which the T cells are intrinsically functionally inactivated following an antigen encounter (Schwartz, R. H., “T cell anergy”, Annu. Rev. Immunol., Vol. 21: 305-334 (2003)) contribute to the downregulation of the immune response. A third mechanism is provided by active suppression of activated T cells by suppressor or regulatory CD4⁺ T (Treg) cells (Reviewed in Kronenberg, M. et al., “Regulation of immunity by self-reactive T cells”, Nature, Vol. 435: 598-604 (2005)). CD4⁺ Tregs that constitutively express the IL-2 receptor alpha (IL-2Ra) chain (CD4⁺CD25⁺) are a naturally occurring T cell subset that are anergic and suppressive (Taams, L. S. et al., “Human anergic/suppressive CD4⁺CD25⁺ T cells: a highly differentiated and apoptosis-prone population”, Eur. J. Immunol. Vol. 31: 1122-1131 (2001)). Human CD4⁺CD25⁺ Tregs, similar to their murine counterpart, are generated in the thymus and are characterized by the ability to suppress proliferation of responder T cells through a cell-cell contact-dependent mechanism, the inability to produce IL-2, and the anergic phenotype in vitro. Human CD4⁺CD25⁺ T cells can be split into suppressive (CD25^{high}) and nonsuppressive (CD25^{low}) cells, according to the level of CD25 expression. A member of the forkhead family of transcription factors, FOXP3, has been shown to be expressed in murine and human CD4⁺CD25⁺ Tregs and appears to be a master gene controlling CD4⁺CD25⁺ Treg development (Battaglia, M. et al., “Rapamycin promotes expansion of functional CD4⁺CD25⁺Foxp3⁺ regulator T cells of both healthy subjects and type 1 diabetic patients”, J. Immunol., Vol. 177: 8338-8347, (2006)). Accordingly, in some embodiments, an increase in immune response may be associated with a lack of activation or proliferation of regulatory T cells.

Cytotoxic T Lymphocytes

CD8⁺ T cells that recognize peptides from proteins produced within the target cell have cytotoxic properties in that they lead to lysis of the target cells. The mechanism of CTL-induced lysis involves the production by the CTL of perforin, a molecule that can insert into the membrane of target cells and promote the lysis of that cell. Perforin-mediated lysis is enhanced by granzymes, a series of enzymes produced by activated CTLs. Many active CTLs also express large amounts of fas ligand on their surface. The interaction of fas ligand on the surface of CTL with fas on the surface of the target cell initiates apoptosis in the target cell, leading to the death of these cells. CTL-mediated lysis appears to be a major mechanism for the destruction of virally infected cells.

Lymphocyte Activation

The term “activation” or “lymphocyte activation” refers to stimulation of lymphocytes by specific antigens, nonspecific mitogens, or allogeneic cells resulting in synthesis of RNA, protein and DNA and production of lymphokines; it is followed by proliferation and differentiation of various effector and memory cells. T-cell activation is dependent on the interaction of the TCR/CD3 complex with its cognate ligand, a peptide bound in the groove of a class I or class II MHC molecule. The molecular events set in motion by receptor engagement are complex. Among the earliest steps appears to be the activation of tyrosine kinases leading to the tyrosine phosphorylation of a set of substrates that control several signaling pathways. These include a set of adapter proteins that link the TCR to the ras pathway, phospholipase Cγ1, the tyrosine phosphorylation of which increases its catalytic activity and engages the inositol phospholipid metabolic pathway, leading to elevation of intracellular free calcium concentration and activation of protein kinase C, and a series of other enzymes that control cellular growth and differentiation. Full responsiveness of a T cell requires, in addition to receptor engagement, an accessory cell-delivered costimulatory activity, e.g., engagement of CD28 on the T cell by CD80 and/or CD86 on the APC.

T-memory Cells

Following the recognition and eradication of pathogens through adaptive immune responses, the vast majority (90-95%) of T cells undergo apoptosis with the remaining cells forming a pool of memory T cells, designated central memory T cells (TCM), effector memory T cells (TEM), and resident memory T cells (TRM) (Clark, R. A., “Resident memory T cells in human health and disease”, Sci. Transl. Med., 7, 269rv1, (2015)).

Compared to standard T cells, these memory T cells are long-lived with distinct phenotypes such as expression of specific surface markers, rapid production of different cytokine profiles, capability of direct effector cell function, and unique homing distribution patterns. Memory T cells exhibit quick reactions upon re-exposure to their respective antigens in order to eliminate the reinfection of the offender and thereby restore balance of the immune system rapidly. Increasing evidence substantiates that autoimmune memory T cells hinder most attempts to treat or cure autoimmune diseases (Clark, R. A., “Resident memory T cells in human health and disease”, Sci. Transl. Med., Vol. 7, 269rv1, (2015)).

### Increasing Immune Activity

The viruses engineered to comprise one or more polynucleotides that promote thanotransmission described herein may increase immune activity in a tissue or subject by increasing the level or activity of any one or more of the immune cells described herein, for example, macrophages, monocytes, dendritic cells, B-cells, T-cells, and CD4+, CD8+ or CD3+ cells (e.g. CD4+, CD8+ or CD3+ T cells) in the tissue or subject. For example, in one embodiment, the virus engineered to comprise one or more polynucleotides that promote thanotransmission is administered in an amount sufficient to increase in a tissue or subject one or more of: the level or activity of macrophages, the level or activity of monocytes, the level or activity of dendritic cells, the level or activity of T-cells, the level or activity of B-cells, and the level or activity of CD4+, CD8+ or CD3+ cells (e.g. CD4+, CD8+ or CD3+ T cells).

In some aspects, the disclosure relates to a method of increasing the level or activity of macrophages, monocytes, B-cells, T-cells and/or dendritic cells in a tissue or subject, comprising administering to the tissue or subject, the virus engineered to comprise one or more polynucleotides that promote thanotransmission, wherein the virus is administered in an amount sufficient to increase the level or activity of macrophages, monocytes, B-cells, T cells and/or dendritic cells relative to a tissue or subject that is not treated with the engineered virus.

In one embodiment, the subject is in need of an increased level or activity of macrophages, monocytes, dendritic cells, B-cells, and/or T-cells,.

In one embodiment, the level or activity of macrophages, monocytes, B-cells, T-cells or dendritic cells is increased by at least 10%, 20%, 30%, 40%, 50%, 60%, 70%, 80%, 90% or 100%, or by at least 2-fold, 4-fold, 6-fold, 8-fold, or 10-fold relative to a tissue or subject that is not treated with the engineered virus.

In some aspects, the disclosure relates to a method of increasing the level or activity of CD4+, CD8+, or CD3+ cells in a tissue or subject, comprising administering to the subject a virus engineered to comprise one or more polynucleotides that promote thanotransmission in an amount sufficient to increase the level or activity of CD4+, CD8+, or CD3+ cells relative to a tissue or subject that is not treated with the engineered virus.

In one embodiment, the subject is in need of an increased level or activity of CD4+, CD8+, or CD3+ cells.

In one embodiment, the level or activity of CD4+, CD8+, or CD3+ cells is increased by at least 10%, 20%, 30%, 40%, 50%, 60%, 70%, 80%, 90% or 100%, or by at least 2-fold, 4-fold, 6-fold, 8-fold, or 10-fold relative to a tissue or subject that is not treated with the engineered virus.

The virus engineered to comprise one or more polynucleotides that promote thanotransmission may also increase immune activity in a cell, tissue or subject by increasing the level or activity of a pro-immune cytokine produced by an immune cell. For example, in some embodiments, the virus engineered to comprise one or more polynucleotides that promote thanotransmission is administered in an amount sufficient to increase in a cell, tissue or subject the level or activity of a pro-immune cytokine produced by an immune cell. In one embodiment, the pro-immune cytokine is selected from IFN-α, IL-1, IL-12, IL-18, IL-2, IL-15, IL-4, IL-6, TNF-α, IL-17 and GMCSF.

In some aspects, the disclosure relates to a method of inducing pro-inflammatory transcriptional responses in the immune cells described herein, e.g. inducing NFkB pathways, interferon IRF signaling, and/or STAT signaling in an immune cell in a tissue or subject, comprising administering to the tissue or subject, the virus engineered to comprise one or more polynucleotides that promote thanotransmission in an amount sufficient to induce pro-inflammatory transcriptional responses in the immune cells NFkB pathways, interferon IRF signaling, and/or STAT signaling in an immune cell.

The virus engineered to comprise one or more polynucleotides that promote thanotransmission may also increase immune activity in a cell, tissue or subject by modulation of signaling through intracellular sensors of nucleic acids, e.g. stimulator of interferon genes (STING).

In some aspects, the disclosure relates to a method of increasing immune activity in a cell, tissue or subject by modulation of signaling through intracellular sensors of nucleic acids, e.g. stimulator of interferon genes (STING), comprising administering to the tissue or subject, a virus engineered to comprise one or more polynucleotides that promote thanotransmission in an amount sufficient to increase immune activity in a cell, tissue or subject by modulation of signaling through intracellular sensors of nucleic acids, e.g. stimulator of interferon genes (STING).

The virus engineered to comprise one or more polynucleotides that promote thanotransmission may also increase immune activity in a cell, tissue or subject by inducing pro-inflammatory transcriptional responses in the immune cells described herein, e.g. inducing nuclear factor kappa-light-chain-enhancer of activated B cells (NFkB) pathways, interferon regulatory factor (IRF) signaling, and/or STAT signaling. For example, in some embodiments, the virus engineered to comprise one or more polynucleotides that promote thanotransmission is administered in an amount sufficient to induce NFkB pathways, interferon IRF signaling, and/or STAT signaling in an immune cell.

In some aspects, the disclosure relates to a method of inducing pro-inflammatory transcriptional responses in the immune cells described herein, e.g. inducing NFkB pathways, interferon IRF signaling, and/or STAT signaling in an immune cell in a tissue or subject, comprising administering to the tissue or subject, a virus engineered to comprise one or more polynucleotides that promote thanotransmission, wherein the virus is administered in an amount sufficient to induce pro-inflammatory transcriptional responses in the immune cells NFkB pathways, interferon IRF signaling, and/or STAT signaling in an immune cell.

The virus engineered to comprise one or more polynucleotides that promote thanotransmission may also increase immune activity in a tissue or subject by induction or modulation of an antibody response. For example, in some embodiments, the virus engineered to comprise one or more polynucleotides that promote thanotransmission is administered in an amount sufficient to induce or modulate an antibody response in the tissue or subject.

In some aspects, the disclosure relates to a method of increasing immune activity in a tissue or subject by induction or modulation of an antibody response in an immune cell in a tissue or subject, comprising administering to the tissue or subject, a virus engineered to comprise one or more polynucleotides that promote thanotransmission, wherein the virus is administered in an amount sufficient to increase immune activity in the tissue or subject relative to a tissue or subject that is not treated with the engineered virus

In some aspects, the disclosure relates to a method of increasing the level or activity of a pro-immune cytokine in a cell, tissue or subject, comprising administering to the cell, tissue or subject a virus engineered to comprise one or more polynucleotides that promote thanotransmission, wherein the virus is administered in an amount sufficient to increase the level or activity of the pro-immune cytokine relative to a cell, tissue or subject that is not treated with the engineered virus.

In one embodiment, the subject is in need of an increased level or activity of a pro-immune cytokine.

In one embodiment, the level or activity of the pro-immune cytokine is increased by at least 10%, 20%, 30%, 40%, 50%, 60%, 70%, 80%, 90% or 100%, or by at least 2-fold, 4-fold, 6-fold, 8-fold, or 10-fold relative to a cell, tissue or subject that is not treated with the engineered virus.

In one embodiment, the pro-immune cytokine is selected from IFN-α, IL-1, IL-12, IL-18, IL-2, IL-15, IL-4, IL-6, TNF-α, IL-17 and GMCSF.

In some embodiments, the methods disclosed herein further include, before administration of the virus engineered to comprise one or more polynucleotides that promote thanotransmission evaluating the cell, tissue or subject for one or more of: the level or activity of macrophages; the level or activity of monocytes; the level or activity of dendritic cells; the level or activity of CD4+ cells, CD8+ cells, or CD3+ cells; the level or activity of T cells; the level or activity of B cells, and the level or activity of a pro-immune cytokine.

In one embodiment, the methods of the invention further include, after administration of the virus engineered to comprise one or more polynucleotides that promote thanotransmission, evaluating the cell, tissue or subject for one or more of: the level or activity of NFkB, IRF or STING; the level or activity of macrophages; the level or activity of monocytes; the level or activity of dendritic cells; the level or activity of CD4+ cells, CD8+ cells or CD3+ cells; the level or activity of T cells; and the level or activity of a pro-immune cytokine.

Methods of measuring the level or activity of NFkB, IRF or STING; the level or activity of macrophages; the level or activity of monocytes; the level or activity of dendritic cells; the level or activity of CD4+ cells, CD8+ cells or CD3+ cells; the level or activity of T cells; and the level or activity of a pro-immune cytokine are known in the art.

For example, the protein level or activity of NFkB, IRF or STING may be measured by suitable techniques known in the art including ELISA, Western blot or in situ hybridization. The level of a nucleic acid (e.g. an mRNA) encoding NFkB, IRF or STING may be measured using suitable techniques known in the art including polymerase chain reaction (PCR) amplification reaction, reverse-transcriptase PCR analysis, quantitative real-time PCR, single-strand conformation polymorphism analysis (SSCP), mismatch cleavage detection, heteroduplex analysis, Northern blot analysis, in situ hybridization, array analysis, deoxyribonucleic acid sequencing, restriction fragment length polymorphism analysis, and combinations or sub-combinations thereof.

Methods for measuring the level and activity of macrophages are described, for example, in Chitu et al., 2011, Curr Protoc Immunol 14: 1-33. The level and activity of monocytes may be measured by flow cytometry, as described, for example, in Henning et al., 2015, Journal of Immunological Methods 423: 78-84. The level and activity of dendritic cells may be measured by flow cytometry, as described, for example in Dixon et al., 2001, Infect Immun. 69(7): 4351-4357. Each of these references is incorporated by reference herein in its entirety.

The level or activity of T cells may be assessed using a human CD4⁺ T-cell-based proliferative assay. For example, cells are labeled with the fluorescent dye 5,6-carboxyfluorescein diacetate succinimidyl ester (CFSE). Those cells that proliferate show a reduction in CFSE fluorescence intensity, which is measured directly by flow cytometry. Alternatively, radioactive thymidine incorporation can be used to assess the rate of growth of the T cells.

In some embodiments, an increase in immune response may be associated with reduced activation of regulatory T cells (Tregs). Functional activity T regs may be assessed using an in vitro Treg suppression assay. Such an assay is described in Collinson and Vignali (Methods Mol Biol. 2011; 707: 21-37, incorporated by reference in its entirety herein).

The level or activity of a pro-immune cytokine may be quantified, for example, in CD8+ T cells. In embodiments, the pro-immune cytokine is selected from interferon alpha (IFN-α), interleukin-1 (IL-1), IL-12, IL-18, IL-2, IL-15, IL-4, IL-6, tumor necrosis factor alpha (TNF-α), IL-17, and granulocyte-macrophage colony-stimulating factor (GMCSF). Quantitation can be carried out using the ELISPOT (enzyme-linked immunospot) technique, that detects T cells that secrete a given cytokine (e.g. IFN-α) in response to an antigenic stimulation. T cells are cultured with antigen-presenting cells in wells which have been coated with, e.g., anti-IFN-α antibodies. The secreted IFN-α is captured by the coated antibody and then revealed with a second antibody coupled to a chromogenic substrate. Thus, locally secreted cytokine molecules form spots, with each spot corresponding to one IFN-α-secreting cell. The number of spots allows one to determine the frequency of IFN-α-secreting cells specific for a given antigen in the analyzed sample. The ELISPOT assay has also been described for the detection of TNF-α, interleukin-4 (IL-4), IL-6, IL-12, and GMCSF.

### VII. Methods of Treating Cancer

As provided herein, infection of a target cell with a virus comprising one or more polynucleotides that promote thanotransmission can activate immune cells (e.g., T cells, B cells, NK cells, etc.) and, therefore, can enhance immune cell functions such as, for example, those involved in immunotherapies for treatment of cancer. Accordingly, in certain aspects, the disclosure relates to a method of treating a cancer in a subject in need thereof, the method comprising administering to the subject a virus engineered to comprise one or more polynucleotides that promote thanotransmission by the cancer cell, wherein the virus is administered to the subject in an amount and for a time sufficient to promote thanotransmission, thereby treating the cancer in the subject.

The ability of cancer cells to harness a range of complex, overlapping mechanisms to prevent the immune system from distinguishing self from non-self represents the fundamental mechanism of cancers to evade immunesurveillance. Mechanism(s) include disruption of antigen presentation, disruption of regulatory pathways controlling T cell activation or inhibition (immune checkpoint regulation), recruitment of cells that contribute to immune suppression (Tregs, MDSC) or release of factors that influence immune activity (IDO, PGE2). (See Harris et al., 2013, J Immunotherapy Cancer 1:12; Chen et al., 2013, Immunity 39:1; Pardoll, et al., 2012, Nature Reviews: Cancer 12:252; and Sharma et al., 2015, Cell 161:205, each of which is incorporated by reference herein in its entirety.)

Cancers for treatment using the methods described herein include, for example, all types of cancer or neoplasm or malignant tumors found in mammals, including, but not limited to: sarcomas, melanomas, carcinomas, leukemias, and lymphomas.

The term “sarcoma” generally refers to a tumor which is made up of a substance like the embryonic connective tissue and is generally composed of closely packed cells embedded in a fibrillar or homogeneous substance. Examples of sarcomas which can be treated with the methods of the invention include, for example, a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, uterine sarcoma, myxoid liposarcoma, leiomyosarcoma, spindle cell sarcoma, desmoplastic sarcoma, and telangiectaltic sarcoma.

The term “melanoma” is taken to mean a tumor arising from the melanocytic system of the skin and other organs. Melanomas which can be treated with the methods of the invention include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, and superficial spreading melanoma.

The term “carcinoma” refers to a malignant new growth made up of epithelial cells tending to infiltrate the surrounding tissues and give rise to metastases. Carcinomas which can be treated with the methods of the invention, as described herein, include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, colon adenocarcinoma of colon, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, merkel cell carcinoma, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, cervical squamous cell carcinoma, tonsil squamous cell carcinoma, and carcinoma villosum. In a particular embodiment, the cancer is renal cell carcinoma.

The term “leukemia” refers to a type of cancer of the blood or bone marrow characterized by an abnormal increase of immature white blood cells called “blasts”. Leukemia is a broad term covering a spectrum of diseases. In turn, it is part of the even broader group of diseases affecting the blood, bone marrow, and lymphoid system, which are all known as hematological neoplasms. Leukemias can be divided into four major classifications, acute lymphocytic (or lymphoblastic) leukemia (ALL), acute myelogenous (or myeloid or non-lymphatic) leukemia (AML), chronic lymphocytic leukemia (CLL), and chronic myelogenous leukemia (CML). Further types of leukemia include Hairy cell leukemia (HCL), T-cell prolymphocytic leukemia (T-PLL), large granular lymphocytic leukemia, and adult T-cell leukemia. In certain embodiments, leukemias include acute leukemias. In certain embodiments, leukemias include chronic leukemias.

The term “lymphoma” refers to a group of blood cell tumors that develop from lymphatic cells. The two main categories of lymphomas are Hodgkin lymphomas (HL) and non-Hodgkin lymphomas (NHL) Lymphomas include any neoplasms of the lymphatic tissues. The main classes are cancers of the lymphocytes, a type of white blood cell that belongs to both the lymph and the blood and pervades both.

In some embodiments, the compositions are used for treatment of various types of solid tumors, for example breast cancer (e.g. triple negative breast cancer), bladder cancer, genitourinary tract cancer, colon cancer, rectal cancer, endometrial cancer, kidney (renal cell) cancer, pancreatic cancer, prostate cancer, thyroid cancer (e.g. papillary thyroid cancer), skin cancer, bone cancer, brain cancer, cervical cancer, liver cancer, stomach cancer, mouth and oral cancers, esophageal cancer, adenoid cystic cancer, neuroblastoma, testicular cancer, uterine cancer, thyroid cancer, head and neck cancer, kidney cancer, lung cancer (e.g. small cell lung cancer, non-small cell lung cancer), mesothelioma, ovarian cancer, sarcoma, stomach cancer, uterine cancer, cervical cancer, medulloblastoma, and vulvar cancer. In certain embodiments, skin cancer includes melanoma, squamous cell carcinoma, and cutaneous T-cell lymphoma (CTCL).

In a particular embodiment, the cancer to be treated may be a cancer that is “immunologically cold”, e.g. a tumor containing few infiltrating T cells, or a cancer that is not recognized and does not provoke a strong response by the immune system, making it difficult to treat with current immunotherapies. For example, in one embodiment, the cancer is selected from the group consisting of melanoma, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, malignant mesothelioma, leukemia, lymphoma, myelodysplasia syndrome, multiple myeloma, transitional cell carcinoma, neuroblastoma, plasma cell neoplasms, Wilm's tumor, and hepatocellular cancer (e.g. hepatocellular carcinoma).

In some embodiments, the cancer to be treated is responsive to an immunotherapy, e.g. an immune checkpoint therapy such as an immune checkpoint inhibitor. In some embodiments, the cancer that is responsive to an immunotherapy is selected from the group consisting of squamous cell head and neck cancer, melanoma, Merkel cell carcinoma, hepatocellular carcinoma, advanced renal cell carcinoma, metastatic microsatellite instability-high (MSI-H) or mismatch repair deficient (dMMR) cancers (e.g. MSI-H or dMMR colorectal cancer), cervical cancer, small cell lung cancer, non-small cell lung cancer, triple negative breast cancer, gastric and esophagogastric junction (GEJ) carcinoma, Hodgkin's lymphoma, Primary mediastinal B-cell lymphoma (PMBCL), and urothelial cancer (e.g. locally advanced or metastatic urothelial cancer).

In some embodiments, the therapies described herein may be administered to a subject that has previously failed treatment for a cancer with another anti-neoplastic (e.g. immunotherapeutic) regimen. A “subject who has failed an anti-neoplastic regimen” is a subject with cancer that does not respond, or ceases to respond to treatment with an anti-neoplastic regimen per RECIST 1.1 criteria, i.e., does not achieve a complete response, partial response, or stable disease in the target lesion; or does not achieve complete response or non-CR/non-PD of non-target lesions, either during or after completion of the anti-neoplastic regimen, either alone or in conjunction with surgery and/or radiation therapy which, when possible, are often clinically indicated in conjunction with anti-neoplastic therapy. The RECIST 1.1 criteria are described, for example, in Eisenhauer et al., 2009, Eur. J. Cancer 45:228-24 (which is incorporated herein by reference in its entirety), and discussed in greater detail below. A failed anti-neoplastic regimen results in, e.g., tumor growth, increased tumor burden, and/or tumor metastasis. A failed anti-neoplastic regimen as used herein includes a treatment regimen that was terminated due to a dose limiting toxicity, e.g., a grade III or a grade IV toxicity that cannot be resolved to allow continuation or resumption of treatment with the anti-neoplastic agent or regimen that caused the toxicity. In one embodiment, the subject has failed treatment with an anti-neoplastic regimen comprising administration of one or more anti-angiogenic agents.

A failed anti-neoplastic regimen includes a treatment regimen that does not result in at least stable disease for all target and non-target lesions for an extended period, e.g., at least 1 month, at least 2 months, at least 3 months, at least 4 months, at least 5 months, at least 6 months, at least 12 months, at least 18 months, or any time period less than a clinically defined cure. A failed anti-neoplastic regimen includes a treatment regimen that results in progressive disease of at least one target lesion during treatment with the anti-neoplastic agent, or results in progressive disease less than 2 weeks, less than 1 month, less than two months, less than 3 months, less than 4 months, less than 5 months, less than 6 months, less than 12 months, or less than 18 months after the conclusion of the treatment regimen, or less than any time period less than a clinically defined cure.

A failed anti-neoplastic regimen does not include a treatment regimen wherein the subject treated for a cancer achieves a clinically defined cure, e.g., 5 years of complete response after the end of the treatment regimen, and wherein the subject is subsequently diagnosed with a distinct cancer, e.g., more than 5 years, more than 6 years, more than 7 years, more than 8 years, more than 9 years, more than 10 years, more than 11 years, more than 12 years, more than 13 years, more than 14 years, or more than 15 years after the end of the treatment regimen.

RECIST criteria are clinically accepted assessment criteria used to provide a standard approach to solid tumor measurement and provide definitions for objective assessment of change in tumor size for use in clinical trials. Such criteria can also be used to monitor response of an individual undergoing treatment for a solid tumor. The RECIST 1.1 criteria are discussed in detail in Eisenhauer et al., 2009, Eur. J. Cancer 45:228-24, which is incorporated herein by reference. Response criteria for target lesions include:

Complete Response (CR): Disappearance of all target lesions. Any pathological lymph nodes (whether target or non-target) must have a reduction in short axis to <10 mm.

Partial Response (PR): At least a 30% decrease in the sum of diameters of target lesion, taking as a reference the baseline sum diameters.

Progressive Diseases (PD): At least a 20% increase in the sum of diameters of target lesions, taking as a reference the smallest sum on the study (this includes the baseline sum if that is the smallest on the study). In addition to the relative increase of 20%, the sum must also demonstrate an absolute increase of at least 5 mm. (Note: the appearance of one or more new lesions is also considered progression.)

Stable Disease (SD): Neither sufficient shrinkage to qualify for PR nor sufficient increase to qualify for PD, taking as a reference the smallest sum diameters while on study.

RECIST 1.1 criteria also consider non-target lesions which are defined as lesions that may be measureable, but need not be measured, and should only be assessed qualitatively at the desired time points. Response criteria for non-target lesions include:

Complete Response (CR): Disappearance of all non-target lesions and normalization of tumor marker levels. All lymph nodes must be non-pathological in size (<10 mm short axis).

Non-CR/Non-PD: Persistence of one or more non-target lesion(s) and/or maintenance of tumor marker level above the normal limits.

Progressive Disease (PD): Unequivocal progression of existing non-target lesions. The appearance of one or more new lesions is also considered progression. To achieve “unequivocal progression” on the basis of non-target disease, there must be an overall level of substantial worsening of non-target disease such that, even in the presence of SD or PR in target disease, the overall tumor burden has increased sufficiently to merit discontinuation of therapy. A modest “increase” in the size of one or more non-target lesions is usually not sufficient to qualify for unequivocal progression status. The designation of overall progression solely on the basis of change in non-target disease in the face of SD or PR in target disease will therefore be extremely rare.

In some embodiments, the pharmaceutical compositions and combination therapies described herein may be administered to a subject having a refractory cancer. A “refractory cancer” is a malignancy for which surgery is ineffective, which is either initially unresponsive to chemo- or radiation therapy, or which becomes unresponsive to chemo- or radiation therapy over time.

The invention further provides methods of inhibiting tumor cell growth in a subject, comprising administering a virus engineered to comprise one or more polynucleotides that promote thanotransmission such that tumor cell growth is inhibited. In certain embodiments, treating cancer comprises extending survival or extending time to tumor progression as compared to a control, e.g. a subject that is not treated with the engineered virus. In certain embodiments, the subject is a human subject. In some embodiments, the subject is identified as having cancer (e.g. a tumor) prior to administration of the first dose of the virus engineered to comprise one or more polynucleotides that promote thanotransmission. In certain embodiments, the subject has cancer (e.g. a tumor) at the time of the first administration of the virus engineered to comprise one or more polynucleotides that promote thanotransmission.

In one embodiment, administration of the virus engineered to comprise one or more polynucleotides that promote thanotransmission results in one or more of, reducing proliferation of cancer cells, reducing metastasis of cancer cells, reducing neovascularization of a tumor, reducing tumor burden, reducing tumor size, weight or volume, inhibiting tumor growth, increased time to progression of the cancer, and/or prolonging the survival time of a subject having an oncological disorder. In certain embodiments, administration of the virus engineered to comprise one or more polynucleotides that promote thanotransmission reduces proliferation of cancer cells, reduces metastasis of cancer cells, reduces neovascularization of a tumor, reduces tumor burden, reduces tumor size, weight or volume, increases time to progression, inhibits tumor growth and/or prolongs the survival time of the subject by at least 1%, 2%, 3%, 4%, 5%, 10%, 20%, 30%, 40%, 50%, 60%, 70%, 80%, 90%, 100%, 200%, 300%, 400% or 500% relative to a corresponding control subject that is not administered the engineered virus. In certain embodiments, administration of the virus engineered to comprise one or more polynucleotides that promote thanotransmission reduces proliferation of cancer cells, reduces metastasis of cancer cells, reduces neovascularization of a tumor, reduces tumor burden, reduces tumor size, weight or volume, increases time to progression, inhibits tumor growth and/or prolongs the survival time of a population of subjects afflicted with an oncological disorder by at least 1%, 2%, 3%, 4%, 5%, 10%, 20%, 30%, 40%, 50%, 60%, 70%, 80%, 90%, 100%, 200%, 300%, 400% or 500% relative to a corresponding population of control subjects afflicted with the oncological disorder that is not administered the engineered virus. In some embodiments, the proliferation of the cancer cells is a hyperproliferation of the cancer cells resulting from a cancer therapy administered to the subject. In some embodiments, administration of the virus engineered to comprise one or more polynucleotides that promote thanotransmission stabilizes the oncological disorder in a subject with a progressive oncological disorder prior to treatment.

### Combination Therapy of an Engineered Virus and Additional Therapeutic Agents

The terms “administering in combination”, “combination therapy”, “co-administering” or “co-administration” may refer to administration of the virus engineered to comprise one or more polynucleotides that promote thanotransmission in combination with one or more additional therapeutic agents. The one or more additional therapeutic agents may be administered prior to, concurrently or substantially concurrently with, subsequently to, or intermittently with administration of the virus engineered to comprise one or more polynucleotides that promote thanotransmission. In certain embodiments, the one or more additional therapeutic agents is administered prior to administration of the virus engineered to comprise one or more polynucleotides that promote thanotransmission. In certain embodiments, the one or more additional therapeutic agents is administered concurrently with the virus engineered to comprise one or more polynucleotides that promote thanotransmission. In certain embodiments, the one or more additional therapeutic agents is administered after administration of the virus engineered to comprise one or more polynucleotides that promote thanotransmission.

The one or more additional therapeutic agents and the virus engineered to comprise one or more polynucleotides that promote thanotransmission can act additively or synergistically. In one embodiment, the one or more additional therapeutic agents and the virus engineered to comprise one or more polynucleotides that promote thanotransmission act synergistically. In some embodiments the synergistic effects are in the treatment of an oncological disorder or an infection. For example, in one embodiment, the combination of the one or more additional therapeutic agents and the virus engineered to comprise one or more polynucleotides that promote thanotransmission improves the durability, i.e. extends the duration, of the immune response against a cancer. In some embodiments, the one or more additional therapeutic agents and the virus engineered to comprise one or more polynucleotides that promote thanotransmission act additively.

### 1. Immune Checkpoint Modulators

In some embodiments, the additional therapeutic agent administered in combination with the virus engineered to comprise one or more polynucleotides that promote thanotransmission is an immune checkpoint modulator of an immune checkpoint molecule. Examples of immune checkpoint molecules include LAG-3 (Triebel et al., 1990, J. Exp. Med. 171: 1393-1405), TIM-3 (Sakuishi et al., 2010, J. Exp. Med. 207: 2187-2194), VISTA (Wang et al., 2011, J. Exp. Med. 208: 577-592), ICOS (Fan et al., 2014, J. Exp. Med. 211: 715-725), OX40 (Curti et al., 2013, Cancer Res. 73: 7189-7198) and 4-1BB (Melero et al., 1997, Nat. Med. 3: 682-685).

Immune checkpoints may be stimulatory immune checkpoints (i.e. molecules that stimulate the immune response) or inhibitory immune checkpoints (i.e. molecules that inhibit immune response). In some embodiments, the immune checkpoint modulator is an antagonist of an inhibitory immune checkpoint. In some embodiments, the immune checkpoint modulator is an agonist of a stimulatory immune checkpoint. In some embodiments, the immune checkpoint modulator is an immune checkpoint binding protein (e.g., an antibody, antibody Fab fragment, divalent antibody, antibody drug conjugate, scFv, fusion protein, bivalent antibody, or tetravalent antibody). In certain embodiments, the immune checkpoint modulator is capable of binding to, or modulating the activity of more than one immune checkpoint. Examples of stimulatory and inhibitory immune checkpoints, and molecules that modulate these immune checkpoints that may be used in the methods of the invention, are provided below.

i. Stimulatory Immune Checkpoint Molecules

CD27 supports antigen-specific expansion of naïve T cells and is vital for the generation of T cell memory (see, e.g., Hendriks et al. (2000) Nat. Immunol. 171 (5): 433-40). CD27 is also a memory marker of B cells (see, e.g., Agematsu et al. (2000) Histol. Histopathol. 15 (2): 573-6. CD27 activity is governed by the transient availability of its ligand, CD70, on lymphocytes and dendritic cells (see, e.g., Borst et al. (2005) Curr. Opin. Immunol. 17 (3): 275-81). Multiple immune checkpoint modulators specific for CD27 have been developed and may be used as disclosed herein. In some embodiments, the immune checkpoint modulator is an agent that modulates the activity and/or expression of CD27. In some embodiments, the immune checkpoint modulator is an agent that binds to CD27 (e.g., an anti-CD27 antibody). In some embodiments, the checkpoint modulator is a CD27 agonist. In some embodiments, the checkpoint modulator is a CD27 antagonist. In some embodiments, the immune checkpoint modulator is an CD27-binding protein (e.g., an antibody). In some embodiments, the immune checkpoint modulator is varlilumab (Celldex Therapeutics). Additional CD27-binding proteins (e.g., antibodies) are known in the art and are disclosed, e.g., in U.S. Pat. Nos. 9,248,183, 9,102,737, 9,169,325, 9,023,999, 8,481,029; U.S. Patent Application Publication Nos. 2016/0185870, 2015/0337047, 2015/0299330, 2014/0112942, 2013/0336976, 2013/0243795, 2013/0183316, 2012/0213771, 2012/0093805, 2011/0274685, 2010/0173324; and PCT Publication Nos. WO 2015/016718, WO 2014/140374, WO 2013/138586, WO 2012/004367, WO 2011/130434, WO 2010/001908, and WO 2008/051424, each of which is incorporated by reference herein.

CD28. Cluster of Differentiation 28 (CD28) is one of the proteins expressed on T cells that provide co-stimulatory signals required for T cell activation and survival. T cell stimulation through CD28 in addition to the T-cell receptor (TCR) can provide a potent signal for the production of various interleukins (IL-6 in particular). Binding with its two ligands, CD80 and CD86, expressed on dendritic cells, prompts T cell expansion (see, e.g., Prasad et al. (1994) Proc. Nat'l. Acad. Sci. USA 91(7): 2834-8). Multiple immune checkpoint modulators specific for CD28 have been developed and may be used as disclosed herein. In some embodiments, the immune checkpoint modulator is an agent that modulates the activity and/or expression of CD28. In some embodiments, the immune checkpoint modulator is an agent that binds to CD28 (e.g., an anti-CD28 antibody). In some embodiments, the checkpoint modulator is an CD28 agonist. In some embodiments, the checkpoint modulator is an CD28 antagonist. In some embodiments, the immune checkpoint modulator is an CD28-binding protein (e.g., an antibody). In some embodiments, the immune checkpoint modulator is selected from the group consisting of TABO8 (TheraMab LLC), lulizumab (also known as BMS-931699, Bristol-Myers Squibb), and FR104 (OSE Immunotherapeutics). Additional CD28-binding proteins (e.g., antibodies) are known in the art and are disclosed, e.g., in U.S. Pat. Nos. 9,119,840, 8,709,414, 9,085,629, 8,034,585, 7,939,638, 8,389,016, 7,585,960, 8,454,959, 8,168,759, 8,785,604, 7,723,482; U.S. Patent Application Publication Nos. 2016/0017039, 2015/0299321, 2015/0150968, 2015/0071916, 2015/0376278, 2013/0078257, 2013/0230540, 2013/0078236, 2013/0109846, 2013/0266577, 2012/0201814, 2012/0082683, 2012/0219553, 2011/0189735, 2011/0097339, 2010/0266605, 2010/0168400, 2009/0246204, 2008/0038273; and PCT Publication Nos. WO 2015198147, WO 2016/05421, WO 2014/1209168, WO 2011/101791, WO 2010/007376, WO 2010/009391, WO 2004/004768, WO 2002/030459, WO 2002/051871, and WO 2002/047721, each of which is incorporated by reference herein.

CD40. Cluster of Differentiation 40 (CD40, also known as TNFRSF5) is found on a variety of immune system cells including antigen presenting cells. CD40L, otherwise known as CD154, is the ligand of CD40 and is transiently expressed on the surface of activated CD4⁺ T cells. CD40 signaling is known to ‘license’ dendritic cells to mature and thereby trigger T-cell activation and differentiation (see, e.g., O'Sullivan et al. (2003) Crit. Rev. Immunol. 23 (1): 83-107. Multiple immune checkpoint modulators specific for CD40 have been developed and may be used as disclosed herein. In some embodiments, the immune checkpoint modulator is an agent that modulates the activity and/or expression of CD40. In some embodiments, the immune checkpoint modulator is an agent that binds to CD40 (e.g., an anti-CD40 antibody). In some embodiments, the checkpoint modulator is a CD40 agonist. In some embodiments, the checkpoint modulator is an CD40 antagonist. In some embodiments, the immune checkpoint modulator is a CD40-binding protein selected from the group consisting of dacetuzumab (Genentech/Seattle Genetics), CP-870,893 (Pfizer), bleselumab (Astellas Pharma), lucatumumab (Novartis), CFZ533 (Novartis; see, e.g., Cordoba et al. (2015) Am. J. Transplant. 15(11): 2825-36), RG7876 (Genentech Inc.), FFP104 (PanGenetics, B.V.), APX005 (Apexigen), BI 655064 (Boehringer Ingelheim), Chi Lob 7/4 (Cancer Research UK; see, e.g., Johnson et al. (2015) Clin. Cancer Res. 21(6): 1321-8), ADC-1013 (BioInvent International), SEA-CD40 (Seattle Genetics), XmAb 5485 (Xencor), PG120 (PanGenetics B.V.), teneliximab (Bristol-Myers Squibb; see, e.g., Thompson et al. (2011) Am. J. Transplant. 11(5): 947-57), and AKH3 (Biogen; see, e.g., International Publication No. WO 2016/028810). Additional CD40-binding proteins (e.g., antibodies) are known in the art and are disclosed, e.g., in U.S. Pat. Nos. 9,234,044, 9,266,956, 9,109,011, 9,090,696, 9,023,360, 9,023,361, 9,221,913, 8,945,564, 8,926,979, 8,828,396, 8,637,032, 8,277,810, 8,088,383, 7,820,170, 7,790,166, 7,445,780, 7,361,345, 8,961,991, 8,669,352, 8,957,193, 8,778,345, 8,591,900, 8,551,485, 8,492,531, 8,362,210, 8,388,971; U.S. Patent Application Publication Nos. 2016/0045597, 2016/0152713, 2016/0075792, 2015/0299329, 2015/0057437 2015/0315282, 2015/0307616, 2014/0099317, 2014/0179907, 2014/0349395, 2014/0234344, 2014/0348836, 2014/0193405, 2014/0120103, 2014/0105907, 2014/0248266, 2014/0093497, 2014/0010812, 2013/0024956, 2013/0023047, 2013/0315900, 2012/0087927, 2012/0263732, 2012/0301488, 2011/0027276, 2011/0104182, 2010/0234578, 2009/0304687, 2009/0181015, 2009/0130715, 2009/0311254, 2008/0199471, 2008/0085531, 2016/0152721, 2015/0110783, 2015/0086991, 2015/0086559, 2014/0341898, 2014/0205602, 2014/0004131, 2013/0011405, 2012/0121585, 2011/0033456, 2011/0002934, 2010/0172912, 2009/0081242, 2009/0130095, 2008/0254026, 2008/0075727, 2009/0304706, 2009/0202531, 2009/0117111, 2009/0041773, 2008/0274118, 2008/0057070, 2007/0098717, 2007/0218060, 2007/0098718, 2007/0110754; and PCT Publication Nos. WO 2016/069919, WO 2016/023960, WO 2016/023875, WO 2016/028810, WO 2015/134988, WO 2015/091853, WO 2015/091655, WO 2014/065403, WO 2014/070934, WO 2014/065402, WO 2014/207064, WO 2013/034904, WO 2012/125569, WO 2012/149356, WO 2012/111762, WO 2012/145673, WO 2011/123489, WO 2010/123012, WO 2010/104761, WO 2009/094391, WO 2008/091954, WO 2007/129895, WO 2006/128103, WO 2005/063289, WO 2005/063981, WO 2003/040170, WO 2002/011763, WO 2000/075348, WO 2013/164789, WO 2012/075111, WO 2012/065950, WO 2009/062054, WO 2007/124299, WO 2007/053661, WO 2007/053767, WO 2005/044294, WO 2005/044304, WO 2005/044306, WO 2005/044855, WO 2005/044854, WO 2005/044305, WO 2003/045978, WO 2003/029296, WO 2002/028481, WO 2002/028480, WO 2002/028904, WO 2002/028905, WO 2002/088186, and WO 2001/024823, each of which is incorporated by reference herein.

CD122. CD122 is the Interleukin-2 receptor beta sub-unit and is known to increase proliferation of CD8⁺ effector T cells. See, e.g., Boyman et al. (2012) Nat. Rev. Immunol. 12 (3): 180-190. Multiple immune checkpoint modulators specific for CD122 have been developed and may be used as disclosed herein. In some embodiments, the immune checkpoint modulator is an agent that modulates the activity and/or expression of CD122. In some embodiments, the immune checkpoint modulator is an agent that binds to CD122 (e.g., an anti-CD122 antibody). In some embodiments, the checkpoint modulator is an CD122 agonist. In some embodiments, the checkpoint modulator is an CD22 agonist. In some embodiments, the immune checkpoint modulator is humanized MiK-Beta-1 (Roche; see, e.g., Morris et al. (2006) Proc Nat'l. Acad. Sci. USA 103(2): 401-6, which is incorporated by reference). Additional CD122-binding proteins (e.g., antibodies) are known in the art and are disclosed, e.g., in U.S. Pat. No. 9,028,830, which is incorporated by reference herein.

OX40. The OX40 receptor (also known as CD134) promotes the expansion of effector and memory T cells. OX40 also suppresses the differentiation and activity of T-regulatory cells, and regulates cytokine production (see, e.g., Croft et al. (2009) Immunol. Rev. 229(1): 173-91). Multiple immune checkpoint modulators specific for OX40 have been developed and may be used as disclosed herein. In some embodiments, the immune checkpoint modulator is an agent that modulates the activity and/or expression of OX40. In some embodiments, the immune checkpoint modulator is an agent that binds to OX40 (e.g., an anti-OX40 antibody). In some embodiments, the checkpoint modulator is an OX40 agonist. In some embodiments, the checkpoint modulator is an OX40 antagonist. In some embodiments, the immune checkpoint modulator is a OX40-binding protein (e.g., an antibody) selected from the group consisting of MEDI6469 (AgonOx/Medimmune), pogalizumab (also known as MOXR0916 and RG7888; Genentech, Inc.), tavolixizumab (also known as MEDI0562; Medimmune), and GSK3174998 (GlaxoSmithKline). Additional OX-40-binding proteins (e.g., antibodies) are known in the art and are disclosed, e.g., in U.S. Pat. Nos. 9,163,085, 9,040,048, 9,006,396, 8,748,585, 8,614,295, 8,551,477, 8,283,450, 7,550,140; U.S. Patent Application Publication Nos. 2016/0068604, 2016/0031974, 2015/0315281, 2015/0132288, 2014/0308276, 2014/0377284, 2014/0044703, 2014/0294824, 2013/0330344, 2013/0280275, 2013/0243772, 2013/0183315, 2012/0269825, 2012/0244076, 2011/0008368, 2011/0123552, 2010/0254978, 2010/0196359, 2006/0281072; and PCT Publication Nos. WO 2014/148895, WO 2013/068563, WO 2013/038191, WO 2013/028231, WO 2010/096418, WO 2007/062245, and WO 2003/106498, each of which is incorporated by reference herein.

GITR. Glucocorticoid-induced TNFR family related gene (GITR) is a member of the tumor necrosis factor receptor (TNFR) superfamily that is constitutively or conditionally expressed on Treg, CD4, and CD8 T cells. GITR is rapidly upregulated on effector T cells following TCR ligation and activation. The human GITR ligand (GITRL) is constitutively expressed on APCs in secondary lymphoid organs and some nonlymphoid tissues. The downstream effect of GITR:GITRL interaction induces attenuation of Treg activity and enhances CD4⁺ T cell activity, resulting in a reversal of Treg-mediated immunosuppression and increased immune stimulation. Multiple immune checkpoint modulators specific for GITR have been developed and may be used as disclosed herein. In some embodiments, the immune checkpoint modulator is an agent that modulates the activity and/or expression of GITR. In some embodiments, the immune checkpoint modulator is an agent that binds to GITR (e.g., an anti-GITR antibody). In some embodiments, the checkpoint modulator is an GITR agonist. In some embodiments, the checkpoint modulator is an GITR antagonist. In some embodiments, the immune checkpoint modulator is a GITR-binding protein (e.g., an antibody) selected from the group consisting of TRX518 (Leap Therapeutics), MK-4166 (Merck & Co.), MEDI-1873 (MedImmune), INCAGN1876 (Agenus/Incyte), and FPA154 (Five Prime Therapeutics). Additional GITR-binding proteins (e.g., antibodies) are known in the art and are disclosed, e.g., in U.S. Pat. Nos. 9,309,321, 9,255,152, 9,255,151, 9,228,016, 9,028,823, 8,709,424, 8,388,967; U.S. Patent Application Publication Nos. 2016/0145342, 2015/0353637, 2015/0064204, 2014/0348841, 2014/0065152, 2014/0072566, 2014/0072565, 2013/0183321, 2013/0108641, 2012/0189639; and PCT Publication Nos. WO 2016/054638, WO 2016/057841, WO 2016/057846, WO 2015/187835, WO 2015/184099, WO 2015/031667, WO 2011/028683, and WO 2004/107618, each of which is incorporated by reference herein.

ICOS. Inducible T-cell costimulator (ICOS, also known as CD278) is expressed on activated T cells. Its ligand is ICOSL, which is expressed mainly on B cells and dendritic cells. ICOS is important in T cell effector function. ICOS expression is up-regulated upon T cell activation (see, e.g., Fan et al. (2014) J. Exp. Med. 211(4): 715-25). Multiple immune checkpoint modulators specific for ICOS have been developed and may be used as disclosed herein. In some embodiments, the immune checkpoint modulator is an agent that modulates the activity and/or expression of ICOS. In some embodiments, the immune checkpoint modulator is an agent that binds to ICOS (e.g., an anti-ICOS antibody). In some embodiments, the checkpoint modulator is an ICOS agonist. In some embodiments, the checkpoint modulator is an ICOS antagonist. In some embodiments, the immune checkpoint modulator is a ICOS-binding protein (e.g., an antibody) selected from the group consisting of MEDI-570 (also known as JMab-136, Medimmune), GSK3359609 (GlaxoSmithKline/INSERM), and JTX-2011 (Jounce Therapeutics). Additional ICOS-binding proteins (e.g., antibodies) are known in the art and are disclosed, e.g., in U.S. Pat. Nos. 9,376,493, 7,998,478, 7,465,445, 7,465,444; U.S. Patent Application Publication Nos. 2015/0239978, 2012/0039874, 2008/0199466, 2008/0279851; and PCT Publication No. WO 2001/087981, each of which is incorporated by reference herein.

4-1BB. 4-1BB (also known as CD137) is a member of the tumor necrosis factor (TNF) receptor superfamily. 4-1BB (CD137) is a type II transmembrane glycoprotein that is inducibly expressed on primed CD4⁺ and CD8⁺ T cells, activated NK cells, DCs, and neutrophils, and acts as a T cell costimulatory molecule when bound to the 4-1BB ligand (4-1BBL) found on activated macrophages, B cells, and DCs. Ligation of the 4-1BB receptor leads to activation of the NF-KB, c-Jun and p38 signaling pathways and has been shown to promote survival of CD8⁺ T cells, specifically, by upregulating expression of the antiapoptotic genes BcL-x(L) and Bfl-1. In this manner, 4-1BB serves to boost or even salvage a suboptimal immune response. Multiple immune checkpoint modulators specific for 4-1BB have been developed and may be used as disclosed herein. In some embodiments, the immune checkpoint modulator is an agent that modulates the activity and/or expression of 4-1BB. In some embodiments, the immune checkpoint modulator is an agent that binds to 4-1BB (e.g., an anti-4-1BB antibody). In some embodiments, the checkpoint modulator is an 4-1BB agonist. In some embodiments, the checkpoint modulator is an 4-1BB antagonist. In some embodiments, the immune checkpoint modulator is a 4-1BB-binding protein is urelumab (also known as BMS-663513; Bristol-Myers Squibb) or utomilumab (Pfizer). In some embodiments, the immune checkpoint modulator is a 4-1BB-binding protein (e.g., an antibody). 4-1BB-binding proteins (e.g., antibodies) are known in the art and are disclosed, e.g., in U.S. Pat. Nos. 9,382,328, 8,716,452, 8,475,790, 8,137,667, 7,829,088, 7,659,384; U.S. Patent Application Publication Nos. 2016/0083474, 2016/0152722, 2014/0193422, 2014/0178368, 2013/0149301, 2012/0237498, 2012/0141494, 2012/0076722, 2011/0177104, 2011/0189189, 2010/0183621, 2009/0068192, 2009/0041763, 2008/0305113, 2008/0008716; and PCT Publication Nos. WO 2016/029073, WO 2015/188047, WO 2015/179236, WO 2015/119923, WO 2012/032433, WO 2012/145183, WO 2011/031063, WO 2010/132389, WO 2010/042433, WO 2006/126835, WO 2005/035584, WO 2004/010947; and Martinez-Forero et al. (2013) J. Immunol. 190(12): 6694-706, and Dubrot et al. (2010) Cancer Immunol. Immunother. 59(8): 1223-33, each of which is incorporated by reference herein.

ii. Inhibitory Immune Checkpoint Molecules

ADORA2A. The adenosine A2A receptor (A2A4) is a member of the G protein-coupled receptor (GPCR) family which possess seven transmembrane alpha helices, and is regarded as an important checkpoint in cancer therapy. A2A receptor can negatively regulate overreactive immune cells (see, e.g., Ohta et al. (2001) Nature 414(6866): 916-20). Multiple immune checkpoint modulators specific for ADORA2A have been developed and may be used as disclosed herein. In some embodiments, the immune checkpoint modulator is an agent that modulates the activity and/or expression of ADORA2A. In some embodiments, the immune checkpoint modulator is an agent that binds to ADORA2A (e.g., an anti-ADORA2A antibody). In some embodiments, the immune checkpoint modulator is a ADORA2A-binding protein (e.g., an antibody). In some embodiments, the checkpoint modulator is an ADORA2A agonist. In some embodiments, the checkpoint modulator is an ADORA2A antagonist. ADORA2A-binding proteins (e.g., antibodies) are known in the art and are disclosed, e.g., in U.S. Patent Application Publication No. 2014/0322236, which is incorporated by reference herein.

B7-H3. B7-H3 (also known as CD276) belongs to the B7 superfamily, a group of molecules that costimulate or down-modulate T-cell responses. B7-H3 potently and consistently down-modulates human T-cell responses (see, e.g., Leitner et al. (2009) Eur. J. Immunol. 39(7): 1754-64). Multiple immune checkpoint modulators specific for B7-H3 have been developed and may be used as disclosed herein. In some embodiments, the immune checkpoint modulator is an agent that modulates the activity and/or expression of B7-H3. In some embodiments, the immune checkpoint modulator is an agent that binds to B7-H3 (e.g., an anti-B7-H3 antibody). In some embodiments, the checkpoint modulator is an B7-H3 agonist. In some embodiments, the checkpoint modulator is an B7-H3 antagonist. In some embodiments, the immune checkpoint modulator is an anti-B7-H3-binding protein selected from the group consisting of DS-5573 (Daiichi Sankyo, Inc.), enoblituzumab (MacroGenics, Inc.), and 8H9 (Sloan Kettering Institute for Cancer Research; see, e.g., Ahmed et al. (2015) J. Biol. Chem. 290(50): 30018-29). In some embodiments, the immune checkpoint modulator is a B7-H3-binding protein (e.g., an antibody). B7-H3-binding proteins (e.g., antibodies) are known in the art and are disclosed, e.g., in U.S. Pat. Nos. 9,371,395, 9,150,656, 9,062,110, 8,802,091, 8,501,471, 8,414,892; U.S. Patent Application Publication Nos. 2015/0352224, 2015/0297748, 2015/0259434, 2015/0274838, 2014/032875, 2014/0161814, 2013/0287798, 2013/0078234, 2013/0149236, 2012/02947960, 2010/0143245, 2002/0102264; PCT Publication Nos. WO 2016/106004, WO 2016/033225, WO 2015/181267, WO 2014/057687, WO 2012/147713, WO 2011/109400, WO 2008/116219, WO 2003/075846, WO 2002/032375; and Shi et al. (2016) Mol. Med. Rep. 14(1): 943-8, each of which is incorporated by reference herein.

B7-H4. B7-H4 (also known as O8E, OV064, and V-set domain-containing T-cell activation inhibitor (VTCN1)), belongs to the B7 superfamily. By arresting cell cycle, B7-H4 ligation of T cells has a profound inhibitory effect on the growth, cytokine secretion, and development of cytotoxicity. Administration of B7-H4Ig into mice impairs antigen-specific T cell responses, whereas blockade of endogenous B7-H4 by specific monoclonal antibody promotes T cell responses (see, e.g., Sica et al. (2003) Immunity 18(6): 849-61). Multiple immune checkpoint modulators specific for B7-H4 have been developed and may be used as disclosed herein. In some embodiments, the immune checkpoint modulator is an agent that modulates the activity and/or expression of B7-H4. In some embodiments, the immune checkpoint modulator is an agent that binds to B7-H4 (e.g., an anti-B7-H4 antibody). In some embodiments, the immune checkpoint modulator is a B7-H4-binding protein (e.g., an antibody). In some embodiments, the checkpoint modulator is an B7-H4 agonist. In some embodiments, the checkpoint modulator is an B7-H4 antagonist. B7-H4-binding proteins (e.g., antibodies) are known in the art and are disclosed, e.g., in U.S. Pat. Nos. 9,296,822, 8,609,816, 8,759,490, 8,323,645; U.S. Patent Application Publication Nos. 2016/0159910, 2016/0017040, 2016/0168249, 2015/0315275, 2014/0134180, 2014/0322129, 2014/0356364, 2014/0328751, 2014/0294861, 2014/0308259, 2013/0058864, 2011/0085970, 2009/0074660, 2009/0208489; and PCT Publication Nos. WO 2016/040724, WO 2016/070001, WO 2014/159835, WO 2014/100483, WO 2014/100439, WO 2013/067492, WO 2013/025779, WO 2009/073533, WO 2007/067991, and WO 2006/104677, each of which is incorporated by reference herein.

BTLA. B and T Lymphocyte Attenuator (BTLA), also known as CD272, has HVEM (Herpesvirus Entry Mediator) as its ligand. Surface expression of BTLA is gradually downregulated during differentiation of human CD8⁺ T cells from the naive to effector cell phenotype, however tumor-specific human CD8⁺ T cells express high levels of BTLA (see, e.g., Derre et al. (2010) J. Clin. Invest. 120 (1): 157-67). Multiple immune checkpoint modulators specific for BTLA have been developed and may be used as disclosed herein. In some embodiments, the immune checkpoint modulator is an agent that modulates the activity and/or expression of BTLA. In some embodiments, the immune checkpoint modulator is an agent that binds to BTLA (e.g., an anti-BTLA antibody). In some embodiments, the immune checkpoint modulator is a BTLA-binding protein (e.g., an antibody). In some embodiments, the checkpoint modulator is an BTLA agonist. In some embodiments, the checkpoint modulator is an BTLA antagonist. BTLA-binding proteins (e.g., antibodies) are known in the art and are disclosed, e.g., in U.S. Pat. Nos. 9,346,882, 8,580,259, 8,563,694, 8,247,537; U.S. Patent Application Publication Nos. 2014/0017255, 2012/0288500, 2012/0183565, 2010/0172900; and PCT Publication Nos. WO 2011/014438, and WO 2008/076560, each of which is incorporated by reference herein.

CTLA-4. Cytotoxic T lymphocyte antigen-4 (CTLA-4) is a member of the immune regulatory CD28-B7 immunoglobulin superfamily and acts on naïve and resting T lymphocytes to promote immunosuppression through both B7-dependent and B7-independent pathways (see, e.g., Kim et al. (2016) J. Immunol. Res., Article ID 4683607, 14 pp.). CTLA-4 is also known as called CD152. CTLA-4 modulates the threshold for T cell activation. See, e.g., Gajewski et al. (2001) J. Immunol. 166(6): 3900-7. Multiple immune checkpoint modulators specific for CTLA-4 have been developed and may be used as disclosed herein. In some embodiments, the immune checkpoint modulator is an agent that modulates the activity and/or expression of CTLA-4. In some embodiments, the immune checkpoint modulator is an agent that binds to CTLA-4 (e.g., an anti-CTLA-4 antibody). In some embodiments, the checkpoint modulator is an CTLA-4 agonist. In some embodiments, the checkpoint modulator is an CTLA-4 antagonist. In some embodiments, the immune checkpoint modulator is a CTLA-4-binding protein (e.g., an antibody) selected from the group consisting of ipilimumab (Yervoy; Medarex/Bristol-Myers Squibb), tremelimumab (formerly ticilimumab; Pfizer/AstraZeneca), JMW-3B3 (University of Aberdeen), and AGEN1884 (Agenus). Additional CTLA-4 binding proteins (e.g., antibodies) are known in the art and are disclosed, e.g., in U.S. Pat. No. 8,697,845; U.S. Patent Application Publication Nos. 2014/0105914, 2013/0267688, 2012/0107320, 2009/0123477; and PCT Publication Nos. WO 2014/207064, WO 2012/120125, WO 2016/015675, WO 2010/097597, WO 2006/066568, and WO 2001/054732, each of which is incorporated by reference herein.

IDO. Indoleamine 2,3-dioxygenase (IDO) is a tryptophan catabolic enzyme with immune-inhibitory properties. Another important molecule is TDO, tryptophan 2,3-dioxygenase. IDO is known to suppress T and NK cells, generate and activate Tregs and myeloid-derived suppressor cells, and promote tumor angiogenesis. Prendergast et al., 2014, Cancer Immunol Immunother. 63 (7): 721-35, which is incorporated by reference herein.

Multiple immune checkpoint modulators specific for IDO have been developed and may be used as disclosed herein. In some embodiments, the immune checkpoint modulator is an agent that modulates the activity and/or expression of IDO. In some embodiments, the immune checkpoint modulator is an agent that binds to IDO (e.g., an IDO binding protein, such as an anti-IDO antibody). In some embodiments, the checkpoint modulator is an IDO agonist. In some embodiments, the checkpoint modulator is an IDO antagonist. In some embodiments, the immune checkpoint modulator is selected from the group consisting of Norharmane, Rosmarinic acid, COX-2 inhibitors, alpha-methyl-tryptophan, and Epacadostat. In one embodiment, the modulator is Epacadostat.

KIR. Killer immunoglobulin-like receptors (KIRs) comprise a diverse repertoire of MHCI binding molecules that negatively regulate natural killer (NK) cell function to protect cells from NK-mediated cell lysis. KIRs are generally expressed on NK cells but have also been detected on tumor specific CTLs. Multiple immune checkpoint modulators specific for KIR have been developed and may be used as disclosed herein. In some embodiments, the immune checkpoint modulator is an agent that modulates the activity and/or expression of KIR. In some embodiments, the immune checkpoint modulator is an agent that binds to KIR (e.g., an anti-KIR antibody). In some embodiments, the immune checkpoint modulator is a KIR-binding protein (e.g., an antibody). In some embodiments, the checkpoint modulator is an KIR agonist. In some embodiments, the checkpoint modulator is an KIR antagonist. In some embodiments the immune checkpoint modulator is lirilumab (also known as BMS-986015; Bristol-Myers Squibb). Additional KIR binding proteins (e.g., antibodies) are known in the art and are disclosed, e.g., in U.S. Pat. Nos. 8,981,065, 9,018,366, 9,067,997, 8,709,411, 8,637,258, 8,614,307, 8,551,483, 8,388,970, 8,119,775; U.S. Patent Application Publication Nos. 2015/0344576, 2015/0376275, 2016/0046712, 2015/0191547, 2015/0290316, 2015/0283234, 2015/0197569, 2014/0193430, 2013/0143269, 2013/0287770, 2012/0208237, 2011/0293627, 2009/0081240, 2010/0189723; and PCT Publication Nos. WO 2016/069589, WO 2015/069785, WO 2014/066532, WO 2014/055648, WO 2012/160448, WO 2012/071411, WO 2010/065939, WO 2008/084106, WO 2006/072625, WO 2006/072626, and WO 2006/003179, each of which is incorporated by reference herein.

LAG-3, Lymphocyte-activation gene 3 (LAG-3, also known as CD223) is a CD4-related transmembrane protein that competitively binds MHC II and acts as a co-inhibitory checkpoint for T cell activation (see, e.g., Goldberg and Drake (2011) Curr. Top. Microbiol. Immunol. 344: 269-78). Multiple immune checkpoint modulators specific for LAG-3 have been developed and may be used as disclosed herein. In some embodiments, the immune checkpoint modulator is an agent that modulates the activity and/or expression of LAG-3. In some embodiments, the immune checkpoint modulator is an agent that binds to LAG-3 (e.g., an anti-PD-1 antibody). In some embodiments, the checkpoint modulator is an LAG-3 agonist. In some embodiments, the checkpoint modulator is an LAG-3 antagonist. In some embodiments, the immune checkpoint modulator is a LAG-3-binding protein (e.g., an antibody) selected from the group consisting of pembrolizumab (Keytruda; formerly lambrolizumab; Merck & Co., Inc.), nivolumab (Opdivo; Bristol-Myers Squibb), pidilizumab (CT-011, CureTech), SHR-1210 (Incyte/Jiangsu Hengrui Medicine Co., Ltd.), MEDI0680 (also known as AMP-514; Amplimmune Inc./Medimmune), PDR001 (Novartis), BGB-A317 (BeiGene Ltd.), TSR-042 (also known as ANB011; AnaptysBio/Tesaro, Inc.), REGN2810 (Regeneron Pharmaceuticals, Inc./Sanofi-Aventis), and PF-06801591 (Pfizer). Additional PD-1-binding proteins (e.g., antibodies) are known in the art and are disclosed, e.g., in U.S. Pat. Nos. 9,181,342, 8,927,697, 7,488,802, 7,029,674; U.S. Patent Application Publication Nos. 2015/0152180, 2011/0171215, 2011/0171220; and PCT Publication Nos. WO 2004/056875, WO 2015/036394, WO 2010/029435, WO 2010/029434, WO 2014/194302, each of which is incorporated by reference herein.

PD-1. Programmed cell death protein 1 (PD-1, also known as CD279 and PDCD1) is an inhibitory receptor that negatively regulates the immune system. In contrast to CTLA-4 which mainly affects naïve T cells, PD-1 is more broadly expressed on immune cells and regulates mature T cell activity in peripheral tissues and in the tumor microenvironment. PD-1 inhibits T cell responses by interfering with T cell receptor signaling. PD-1 has two ligands, PD-L1 and PD-L2. Multiple immune checkpoint modulators specific for PD-1 have been developed and may be used as disclosed herein. In some embodiments, the immune checkpoint modulator is an agent that modulates the activity and/or expression of PD-1. In some embodiments, the immune checkpoint modulator is an agent that binds to PD-1 (e.g., an anti-PD-1 antibody). In some embodiments, the checkpoint modulator is an PD-1 agonist. In some embodiments, the checkpoint modulator is an PD-1 antagonist. In some embodiments, the immune checkpoint modulator is a PD-1-binding protein (e.g., an antibody) selected from the group consisting of pembrolizumab (Keytruda; formerly lambrolizumab; Merck & Co., Inc.), nivolumab (Opdivo; Bristol-Myers Squibb), pidilizumab (CT-011, CureTech), SHR-1210 (Incyte/Jiangsu Hengrui Medicine Co., Ltd.), MEDI0680 (also known as AMP-514; Amplimmune Inc./Medimmune), PDR001 (Novartis), BGB-A317 (BeiGene Ltd.), TSR-042 (also known as ANB011; AnaptysBio/Tesaro, Inc.), REGN2810 (Regeneron Pharmaceuticals, Inc./Sanofi-Aventis), and PF-06801591 (Pfizer). Additional PD-1-binding proteins (e.g., antibodies) are known in the art and are disclosed, e.g., in U.S. Pat. Nos. 9,181,342, 8,927,697, 7,488,802, 7,029,674; U.S. Patent Application Publication Nos. 2015/0152180, 2011/0171215, 2011/0171220; and PCT Publication Nos. WO 2004/056875, WO 2015/036394, WO 2010/029435, WO 2010/029434, WO 2014/194302, each of which is incorporated by reference herein.

PD-L1/PD-L2. PD ligand 1 (PD-L1, also known as B7-H1) and PD ligand 2 (PD-L2, also known as PDCD1LG2, CD273, and B7-DC) bind to the PD-1 receptor. Both ligands belong to the same B7 family as the B7-1 and B7-2 proteins that interact with CD28 and CTLA-4. PD-L1 can be expressed on many cell types including, for example, epithelial cells, endothelial cells, and immune cells. Ligation of PDL-1 decreases IFNγ, TNFα, and IL-2 production and stimulates production of IL10, an anti-inflammatory cytokine associated with decreased T cell reactivity and proliferation as well as antigen-specific T cell anergy. PDL-2 is predominantly expressed on antigen presenting cells (APCs). PDL2 ligation also results in T cell suppression, but where PDL-1-PD-1 interactions inhibits proliferation via cell cycle arrest in the G1/G2 phase, PDL2-PD-1 engagement has been shown to inhibit TCR-mediated signaling by blocking B7:CD28 signals at low antigen concentrations and reducing cytokine production at high antigen concentrations. Multiple immune checkpoint modulators specific for PD-L1 and PD-L2 have been developed and may be used as disclosed herein.

In some embodiments, the immune checkpoint modulator is an agent that modulates the activity and/or expression of PD-L1. In some embodiments, the immune checkpoint modulator is an agent that binds to PD-L1 (e.g., an anti-PD-L1 antibody). In some embodiments, the checkpoint modulator is an PD-L1 agonist. In some embodiments, the checkpoint modulator is an PD-L1 antagonist. In some embodiments, the immune checkpoint modulator is a PD-L1-binding protein (e.g., an antibody or a Fc-fusion protein) selected from the group consisting of durvalumab (also known as MEDI-4736; AstraZeneca/Celgene Corp./Medimmune), atezolizumab (Tecentriq; also known as MPDL3280A and RG7446; Genetech Inc.), avelumab (also known as MSB0010718C; Merck Serono/AstraZeneca); MDX-1105 (Medarex/Bristol-Meyers Squibb), AMP-224 (Amplimmune, GlaxoSmithKline), LY3300054 (Eli Lilly and Co.). Additional PD-L1-binding proteins are known in the art and are disclosed, e.g., in U.S. Patent Application Publication Nos. 2016/0084839, 2015/0355184, 2016/0175397, and PCT Publication Nos. WO 2014/100079, WO 2016/030350, WO2013181634, each of which is incorporated by reference herein.

In some embodiments, the immune checkpoint modulator is an agent that modulates the activity and/or expression of PD-L2. In some embodiments, the immune checkpoint modulator is an agent that binds to PD-L2 (e.g., an anti-PD-L2 antibody). In some embodiments, the checkpoint modulator is an PD-L2 agonist. In some embodiments, the checkpoint modulator is an PD-L2 antagonist. PD-L2-binding proteins (e.g., antibodies) are known in the art and are disclosed, e.g., in U.S. Pat. Nos. 9,255,147, 8,188,238; U.S. Patent Application Publication Nos. 2016/0122431, 2013/0243752, 2010/0278816, 2016/0137731, 2015/0197571, 2013/0291136, 2011/0271358; and PCT Publication Nos. WO 2014/022758, and WO 2010/036959, each of which is incorporated by reference herein.

TIM-3. T cell immunoglobulin mucin 3 (TIM-3, also known as Hepatitis A virus cellular receptor (HAVCR2)) is a type I glycoprotein receptor that binds to S-type lectin galectin-9 (Gal-9). TIM-3, is a widely expressed ligand on lymphocytes, liver, small intestine, thymus, kidney, spleen, lung, muscle, reticulocytes, and brain tissue. Tim-3 was originally identified as being selectively expressed on IFN-γ-secreting Th1 and Tc1 cells (Monney et al. (2002) Nature 415: 536-41). Binding of Gal-9 by the TIM-3 receptor triggers downstream signaling to negatively regulate T cell survival and function. Multiple immune checkpoint modulators specific for TIM-3 have been developed and may be used as disclosed herein. In some embodiments, the immune checkpoint modulator is an agent that modulates the activity and/or expression of TIM-3. In some embodiments, the immune checkpoint modulator is an agent that binds to TIM-3 (e.g., an anti-TIM-3 antibody). In some embodiments, the checkpoint modulator is an TIM-3 agonist. In some embodiments, the checkpoint modulator is an TIM-3 antagonist. In some embodiments, the immune checkpoint modulator is an anti-TIM-3 antibody selected from the group consisting of TSR-022 (AnaptysBio/Tesaro, Inc.) and MGB453 (Novartis). Additional TIM-3 binding proteins (e.g., antibodies) are known in the art and are disclosed, e.g., in U.S. Pat. Nos. 9,103,832, 8,552,156, 8,647,623, 8,841,418; U.S. Patent Application Publication Nos. 2016/0200815, 2015/0284468, 2014/0134639, 2014/0044728, 2012/0189617, 2015/0086574, 2013/0022623; and PCT Publication Nos. WO 2016/068802, WO 2016/068803, WO 2016/071448, WO 2011/155607, and WO 2013/006490, each of which is incorporated by reference herein.

VISTA. V-domain Ig suppressor of T cell activation (VISTA, also known as Platelet receptor Gi24) is an Ig super-family ligand that negatively regulates T cell responses. See, e.g., Wang et al., 2011, J. Exp. Med. 208: 577-92. VISTA expressed on APCs directly suppresses CD4⁺ and CD8⁺ T cell proliferation and cytokine production (Wang et al. (2010) J Exp Med. 208(3): 577-92). Multiple immune checkpoint modulators specific for VISTA have been developed and may be used as disclosed herein. In some embodiments, the immune checkpoint modulator is an agent that modulates the activity and/or expression of VISTA. In some embodiments, the immune checkpoint modulator is an agent that binds to VISTA (e.g., an anti-VISTA antibody). In some embodiments, the checkpoint modulator is an VISTA agonist. In some embodiments, the checkpoint modulator is an VISTA antagonist. In some embodiments, the immune checkpoint modulator is a VISTA-binding protein (e.g., an antibody) selected from the group consisting of TSR-022 (AnaptysBio/Tesaro, Inc.) and MGB453 (Novartis). VISTA-binding proteins (e.g., antibodies) are known in the art and are disclosed, e.g., in U.S. Patent Application Publication Nos. 2016/0096891, 2016/0096891; and PCT Publication Nos. WO 2014/190356, WO 2014/197849, WO 2014/190356 and WO 2016/094837, each of which is incorporated by reference herein.

Methods are provided for the treatment of oncological disorders by administering a virus engineered to comprise one or more polynucleotides that promote thanotransmission by a target cell in combination with at least one immune checkpoint modulator to a subject. In certain embodiments, the immune checkpoint modulator stimulates the immune response of the subject. For example, in some embodiments, the immune checkpoint modulator stimulates or increases the expression or activity of a stimulatory immune checkpoint (e.g. CD27, CD28, CD40, CD122, OX40, GITR, ICOS, or 4-1BB). In some embodiments, the immune checkpoint modulator inhibits or decreases the expression or activity of an inhibitory immune checkpoint (e.g. A2A4, B7-H3, B7-H4, BTLA, CTLA-4, IDO, KIR, LAG3, PD-1, PD-L1, PD-L2, TIM-3 or VISTA).

In certain embodiments the immune checkpoint modulator targets an immune checkpoint molecule selected from the group consisting of CD27, CD28, CD40, CD122, OX40, GITR, ICOS, 4-1BB, A2A4, B7-H3, B7-H4, BTLA, CTLA-4, IDO, KIR, LAG3, PD-1, PD-L1, PD-L2, TIM-3 and VISTA. In certain embodiments the immune checkpoint modulator targets an immune checkpoint molecule selected from the group consisting of CD27, CD28, CD40, CD122, OX40, GITR, ICOS, 4-1BB, A2A4, B7-H3, B7-H4, BTLA, IDO, KIR, LAG3, PD-1, PD-L1, PD-L2, TIM-3 and VISTA. In a particular embodiment, the immune checkpoint modulator targets an immune checkpoint molecule selected from the group consisting of CTLA-4, PD-L1 and PD-1. In a further particular embodiment the immune checkpoint modulator targets an immune checkpoint molecule selected from PD-L1 and PD-1.

In some embodiments, more than one (e.g. 2, 3, 4, 5 or more) immune checkpoint modulator is administered to the subject. Where more than one immune checkpoint modulator is administered, the modulators may each target a stimulatory immune checkpoint molecule, or each target an inhibitory immune checkpoint molecule. In other embodiments, the immune checkpoint modulators include at least one modulator targeting a stimulatory immune checkpoint and at least one immune checkpoint modulator targeting an inhibitory immune checkpoint molecule. In certain embodiments, the immune checkpoint modulator is a binding protein, for example, an antibody. The term “binding protein”, as used herein, refers to a protein or polypeptide that can specifically bind to a target molecule, e.g. an immune checkpoint molecule. In some embodiments the binding protein is an antibody or antigen binding portion thereof, and the target molecule is an immune checkpoint molecule. In some embodiments the binding protein is a protein or polypeptide that specifically binds to a target molecule (e.g., an immune checkpoint molecule). In some embodiments the binding protein is a ligand. In some embodiments, the binding protein is a fusion protein. In some embodiments, the binding protein is a receptor. Examples of binding proteins that may be used in the methods of the invention include, but are not limited to, a humanized antibody, an antibody Fab fragment, a divalent antibody, an antibody drug conjugate, a scFv, a fusion protein, a bivalent antibody, and a tetravalent antibody.

The term “antibody”, as used herein, refers to any immunoglobulin (Ig) molecule comprised of four polypeptide chains, two heavy (H) chains and two light (L) chains, or any functional fragment, mutant, variant, or derivation thereof. Such mutant, variant, or derivative antibody formats are known in the art. In a full-length antibody, each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. Immunoglobulin molecules can be of any type (e.g., IgG, IgE, IgM, IgD, IgA and IgY), class (e.g., IgG 1, IgG2, IgG 3, IgG4, IgA1 and IgA2) or subclass. In some embodiments, the antibody is a full-length antibody. In some embodiments, the antibody is a murine antibody. In some embodiments, the antibody is a human antibody. In some embodiments, the antibody is a humanized antibody. In other embodiments, the antibody is a chimeric antibody. Chimeric and humanized antibodies may be prepared by methods well known to those of skill in the art including CDR grafting approaches (see, e.g., U.S. Pat. Nos. 5,843,708; 6,180,370; 5,693,762; 5,585,089; and 5,530,101), chain shuffling strategies (see, e.g., U.S. Pat. No. 5,565,332; Rader et al. (1998) PROC. NAT'L. ACAD. SCI. USA 95: 8910-8915), molecular modeling strategies (U.S. Pat. No. 5,639,641), and the like.

The term “antigen-binding portion” of an antibody (or simply “antibody portion”), as used herein, refers to one or more fragments of an antibody that retain the ability to specifically bind to an antigen. It has been shown that the antigen-binding function of an antibody can be performed by fragments of a full-length antibody. Such antibody embodiments may also be bispecific, dual specific, or multi-specific formats; specifically binding to two or more different antigens. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al. (1989) NATURE 341: 544-546; and WO 90/05144 A1, the contents of which are herein incorporated by reference), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) SCIENCE 242:423-426; and Huston et al. (1988) PROC. NAT'L. ACAD. SCI. USA 85:5879-5883). Such single chain antibodies are also intended to be encompassed within the term “antigen-binding portion” of an antibody. Other forms of single chain antibodies, such as diabodies are also encompassed. Antigen binding portions can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005).

As used herein, the term “CDR” refers to the complementarity determining region within antibody variable sequences. There are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., SEQUENCES OF PROTEINS OF IMMUNOLOGICAL INTEREST (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers found that certain sub-portions within Kabat CDRs adopt nearly identical peptide backbone conformations, despite having great diversity at the level of amino acid sequence (Chothia et al. (1987) J. MOL. BIOL. 196: 901-917, and Chothia et al. (1989) NATURE 342: 877-883). These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan et al. (1995) FASEB J. 9: 133-139, and MacCallum et al. (1996) J. MOL. BIOL. 262(5): 732-45. Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

The term “humanized antibody”, as used herein refers to non-human (e.g., murine) antibodies that are chimeric immunoglobulins, immunoglobulin chains, or fragments thereof (such as Fv, Fab, Fab′, F(ab′)2 or other antigen-binding subsequences of antibodies) which contain minimal sequence derived from a non-human immunoglobulin. For the most part, humanized antibodies and antibody fragments thereof are human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al. (1986) NATURE 321: 522-525; Reichmann et al. (1988) NATURE 332: 323-329; and Presta (1992) CURR. OP. STRUCT. BIOL. 2: 593-596, each of which is incorporated by reference herein in its entirety.

The term “immunoconjugate” or “antibody drug conjugate” as used herein refers to the linkage of an antibody or an antigen binding fragment thereof with another agent, such as a chemotherapeutic agent, a toxin, an immunotherapeutic agent, an imaging probe, and the like. The linkage can be covalent bonds, or non-covalent interactions such as through electrostatic forces. Various linkers, known in the art, can be employed in order to form the immunoconjugate. Additionally, the immunoconjugate can be provided in the form of a fusion protein that may be expressed from a polynucleotide encoding the immunoconjugate. Translation of the fusion gene results in a single protein with functional properties derived from each of the original proteins.

A “bivalent antibody” refers to an antibody or antigen-binding fragment thereof that comprises two antigen-binding sites. The two antigen binding sites may bind to the same antigen, or they may each bind to a different antigen, in which case the antibody or antigen-binding fragment is characterized as “bispecific.” A “tetravalent antibody” refers to an antibody or antigen-binding fragment thereof that comprises four antigen-binding sites. In certain embodiments, the tetravalent antibody is bispecific. In certain embodiments, the tetravalent antibody is multispecific, i.e. binding to more than two different antigens.

Fab (fragment antigen binding) antibody fragments are immunoreactive polypeptides comprising monovalent antigen-binding domains of an antibody composed of a polypeptide consisting of a heavy chain variable region (VH) and heavy chain constant region 1 (CH1) portion and a poly peptide consisting of a light chain variable (VL) and light chain constant (CL) portion, in which the CL and CH1 portions are bound together, preferably by a disulfide bond between Cys residues.

Immune checkpoint modulator antibodies include, but are not limited to, at least 4 major categories: i) antibodies that block an inhibitory pathway directly on T cells or natural killer (NK) cells (e.g., PD-1 targeting antibodies such as nivolumab and pembrolizumab, antibodies targeting TIM-3, and antibodies targeting LAG-3, 2B4, CD160, A2aR, BTLA, CGEN-15049, and KIR), ii) antibodies that activate stimulatory pathways directly on T cells or NK cells (e.g., antibodies targeting OX40, GITR, and 4-1BB), iii) antibodies that block a suppressive pathway on immune cells or relies on antibody-dependent cellular cytotoxicity to deplete suppressive populations of immune cells (e.g., CTLA-4 targeting antibodies such as ipilimumab, antibodies targeting VISTA, and antibodies targeting PD-L2, Gr1, and Ly6G), and iv) antibodies that block a suppressive pathway directly on cancer cells or that rely on antibody-dependent cellular cytotoxicity to enhance cytotoxicity to cancer cells (e.g., rituximab, antibodies targeting PD-L1, and antibodies targeting B7-H3, B7-H4, Gal-9, and MUC1). Examples of checkpoint inhibitors include, e.g., an inhibitor of CTLA-4, such as ipilimumab or tremelimumab; an inhibitor of the PD-1 pathway such as an anti-PD-1, anti-PD-L1 or anti-PD-L2 antibody. Exemplary anti-PD-1 antibodies are described in WO 2006/121168, WO 2008/156712, WO 2012/145493, WO 2009/014708 and WO 2009/114335. Exemplary anti-PD-L1 antibodies are described in WO 2007/005874, WO 2010/077634 and WO 2011/066389, and exemplary anti-PD-L2 antibodies are described in WO 2004/007679.

In a particular embodiment, the immune checkpoint modulator is a fusion protein, for example, a fusion protein that modulates the activity of an immune checkpoint modulator.

In one embodiment, the immune checkpoint modulator is a therapeutic nucleic acid molecule, for example a nucleic acid that modulates the expression of an immune checkpoint protein or mRNA. Nucleic acid therapeutics are well known in the art. Nucleic acid therapeutics include both single stranded and double stranded (i.e., nucleic acid therapeutics having a complementary region of at least 15 nucleotides in length) nucleic acids that are complementary to a target sequence in a cell. In certain embodiments, the nucleic acid therapeutic is targeted against a nucleic acid sequence encoding an immune checkpoint protein.

Antisense nucleic acid therapeutic agents are single stranded nucleic acid therapeutics, typically about 16 to 30 nucleotides in length, and are complementary to a target nucleic acid sequence in the target cell, either in culture or in an organism.

In another aspect, the agent is a single-stranded antisense RNA molecule. An antisense RNA molecule is complementary to a sequence within the target mRNA. Antisense RNA can inhibit translation in a stoichiometric manner by base pairing to the mRNA and physically obstructing the translation machinery, see Dias, N. et al., (2002) Mol Cancer Ther 1:347-355. The antisense RNA molecule may have about 15-30 nucleotides that are complementary to the target mRNA. Patents directed to antisense nucleic acids, chemical modifications, and therapeutic uses include, for example: U.S. Pat. No. 5,898,031 related to chemically modified RNA-containing therapeutic compounds; U.S. Pat. No. 6,107,094 related methods of using these compounds as therapeutic agents; U.S. Pat. No. 7,432,250 related to methods of treating patients by administering single-stranded chemically modified RNA-like compounds; and U.S. Pat. No. 7,432,249 related to pharmaceutical compositions containing single-stranded chemically modified RNA-like compounds. U.S. Pat. No. 7,629,321 is related to methods of cleaving target mRNA using a single-stranded oligonucleotide having a plurality of RNA nucleosides and at least one chemical modification. The entire contents of each of the patents listed in this paragraph are incorporated herein by reference.

Nucleic acid therapeutic agents for use in the methods of the invention also include double stranded nucleic acid therapeutics. An “RNAi agent,” “double stranded RNAi agent,” double-stranded RNA (dsRNA) molecule, also referred to as “dsRNA agent,” “dsRNA”, “siRNA”, “iRNA agent,” as used interchangeably herein, refers to a complex of ribonucleic acid molecules, having a duplex structure comprising two anti-parallel and substantially complementary, as defined below, nucleic acid strands. As used herein, an RNAi agent can also include dsiRNA (see, e.g., US Patent publication 20070104688, incorporated herein by reference). In general, the majority of nucleotides of each strand are ribonucleotides, but as described herein, each or both strands can also include one or more non-ribonucleotides, e.g., a deoxyribonucleotide and/or a modified nucleotide. In addition, as used in this specification, an “RNAi agent” may include ribonucleotides with chemical modifications; an RNAi agent may include substantial modifications at multiple nucleotides. Such modifications may include all types of modifications disclosed herein or known in the art. Any such modifications, as used in a siRNA type molecule, are encompassed by “RNAi agent” for the purposes of this specification and claims. The RNAi agents that are used in the methods of the invention include agents with chemical modifications as disclosed, for example, in WO/2012/037254, and WO 2009/073809, the entire contents of each of which are incorporated herein by reference.

Immune checkpoint modulators may be administered at appropriate dosages to treat the oncological disorder, for example, by using standard dosages. One skilled in the art would be able, by routine experimentation, to determine what an effective, non-toxic amount of an immune checkpoint modulator would be for the purpose of treating oncological disorders. Standard dosages of immune checkpoint modulators are known to a person skilled in the art and may be obtained, for example, from the product insert provided by the manufacturer of the immune checkpoint modulator. Examples of standard dosages of immune checkpoint modulators are provided in Table 8 below. In other embodiments, the immune checkpoint modulator is administered at a dosage that is different (e.g. lower) than the standard dosages of the immune checkpoint modulator used to treat the oncological disorder under the standard of care for treatment for a particular oncological disorder.

TABLE 8
Exemplary Standard Dosages of Immune Checkpoint Modulators

| | Immune | |
|---|---|---|
| | Checkpoint | |
| Immune Checkpoint | Molecule | |
| Modulator | Targeted | Exemplary Standard Dosage |
| Ipilimumab (Yervoy ™) | CTLA-4 | 3 mg/kg administered intravenously over 90 |
| | | minutes every 3 weeks for a total of 4 doses |
| Pembrolizumab (Keytruda ™) | PD-1 | 2 mg/kg administered as an intravenous |
| | | infusion over 30 minutes every 3 weeks until |
| | | disease progression or unacceptable toxicity |
| Atezolizumab (Tecentriq ™) | PD-L1 | 1200 mg administered as an intravenous |
| | | infusion over 60 minutes every 3 weeks |

In certain embodiments, the administered dosage of the immune checkpoint modulator is 5%, 10%, 20%, 30%, 40%, 50%, 60%, 70%, 80%, or 90% lower than the standard dosage of the immune checkpoint modulator for a particular oncological disorder. In certain embodiments, the dosage administered of the immune checkpoint modulator is 95%, 90%, 85%, 80%, 75%, 70%, 65%, 60%, 55%, 50%, 45%, 40%, 35%, 30%, 25%, 20%, 15%, 10% or 5% of the standard dosage of the immune checkpoint modulator for a particular oncological disorder. In one embodiment, where a combination of immune checkpoint modulators are administered, at least one of the immune checkpoint modulators is administered at a dose that is lower than the standard dosage of the immune checkpoint modulator for a particular oncological disorder. In one embodiment, where a combination of immune checkpoint modulators are administered, at least two of the immune checkpoint modulators are administered at a dose that is lower than the standard dosage of the immune checkpoint modulators for a particular oncological disorder. In one embodiment, where a combination of immune checkpoint modulators are administered, at least three of the immune checkpoint modulators are administered at a dose that is lower than the standard dosage of the immune checkpoint modulators for a particular oncological disorder. In one embodiment, where a combination of immune checkpoint modulators are administered, all of the immune checkpoint modulators are administered at a dose that is lower than the standard dosage of the immune checkpoint modulators for a particular oncological disorder.

Additional immunotherapeutics that may be administered in combination with the virus engineered to comprise one or more polynucleotides that promote thanotransmission by a target cell include, but are not limited to, Toll-like receptor (TLR) agonists, cell-based therapies, cytokines and cancer vaccines.

### 2. TLR Agonists

TLRs are single membrane-spanning non-catalytic receptors that recognize structurally conserved molecules derived from microbes. TLRs together with the Interleukin-1 receptor form a receptor superfamily, known as the “Interleukin-1 Receptor/Toll-Like Receptor Superfamily.” Members of this family are characterized structurally by an extracellular leucine-rich repeat (LRR) domain, a conserved pattern of juxtamembrane cysteine residues, and an intracytoplasmic signaling domain that forms a platform for downstream signaling by recruiting TIR domain-containing adapters including MyD88, TIR domain-containing adaptor (TRAP), and TIR domain-containing adaptor inducing IFNβ (TRIF) (O'Neill et al., 2007, Nat Rev Immunol 7, 353).

The TLRs include TLR1, TLR2, TLR3, TLR4, TLR5, TLR6, TLR7, TLR8, TLR9, and TLR10. TLR2 mediates cellular responses to a large number of microbial products including peptidoglycan, bacterial lipopeptides, lipoteichoic acid, mycobacterial lipoarabinomannan and yeast cell wall components. TLR4 is a transmembrane protein which belongs to the pattern recognition receptor (PRR) family. Its activation leads to an intracellular signaling pathway NF-κB and inflammatory cytokine production which is responsible for activating the innate immune system. TLR5 is known to recognize bacterial flagellin from invading mobile bacteria, and has been shown to be involved in the onset of many diseases, including inflammatory bowel disease.

TLR agonists are known in the art and are described, for example, in US2014/0030294, which is incorporated by reference herein in its entirety. Exemplary TLR2 agonists include mycobacterial cell wall glycolipids, lipoarabinomannan (LAM) and mannosylated phosphatidylinositol (PIIM), MALP-2 and Pam3Cys and synthetic variants thereof. Exemplary TLR4 agonists include lipopolysaccharide or synthetic variants thereof (e.g., MPL and RC529) and lipid A or synthetic variants thereof (e.g., aminoalkyl glucosaminide 4-phosphates). See, e.g., Cluff et al., 2005, Infection and Immunity, p. 3044-3052:73; Lembo et al., 2008, The Journal of Immunology 180, 7574-7581; and Evans et al., 2003, Expert Rev Vaccines 2:219-29. Exemplary TLR5 agonists include flagellin or synthetic variants thereof (e.g., A pharmacologically optimized TLR5 agonist with reduced immunogenicity (such as CBLB502) made by deleting portions of flagellin that are non-essential for TLR5 activation).

Additional TLR agonists include Coley's toxin and Bacille Calmette-Guérin (BCG). Coley's toxin is a mixture consisting of killed bacteria of species Streptococcus pyogenes and Serratia marcescens. See Taniguchi et al., 2006, Anticancer Res. 26 (6A): 3997-4002. BCG is prepared from a strain of the attenuated live bovine tuberculosis bacillus, Mycobacterium bovis. See Venkataswamy et al., 2012, Vaccine. 30 (6): 1038-1049.

### 3. Cell Based Therapies

Cell-based therapies for the treatment of cancer include administration of immune cells (e.g. T cells, tumor-infiltrating lymphocytes (TILs), Natural Killer cells, and dendritic cells) to a subject. In autologous cell-based therapy, the immune cells are derived from the same subject to which they are administered. In allogeneic cell-based therapy, the immune cells are derived from one subject and administered to a different subject. The immune cells may be activated, for example, by treatment with a cytokine, before administration to the subject. In some embodiments, the immune cells are genetically modified before administration to the subject, for example, as in chimeric antigen receptor (CAR) T cell immunotherapy.

In some embodiments, the cell-based therapy includes an adoptive cell transfer (ACT). ACT typically consists of three parts: lympho-depletion, cell administration, and therapy with high doses of IL-2. Types of cells that may be administered in ACT include tumor infiltrating lymphocytes (TILs), T cell receptor (TCR)-transduced T cells, and chimeric antigen receptor (CAR) T cells.

Tumor-infiltrating lymphocytes are immune cells that have been observed in many solid tumors, including breast cancer. They are a population of cells comprising a mixture of cytotoxic T cells and helper T cells, as well as B cells, macrophages, natural killer cells, and dendritic cells. The general procedure for autologous TIL therapy is as follows: (1) a resected tumor is digested into fragments; (2) each fragment is grown in IL-2 and the lymphocytes proliferate destroying the tumor; (3) after a pure population of lymphocytes exists, these lymphocytes are expanded; and (4) after expansion up to 10¹¹ cells, lymphocytes are infused into the patient. See Rosenberg et al., 2015, Science 348(6230):62-68, which is incorporated by reference herein in its entirety.

TCR-transduced T cells are generated via genetic induction of tumor-specific TCRs. This is often done by cloning the particular antigen-specific TCR into a retroviral backbone. Blood is drawn from patients and peripheral blood mononuclear cells (PBMCs) are extracted. PBMCs are stimulated with CD3 in the presence of IL-2 and then transduced with the retrovirus encoding the antigen-specific TCR. These transduced PBMCs are expanded further in vitro and infused back into patients. See Robbins et al., 2015, Clinical Cancer Research 21(5):1019-1027, which is incorporated by reference herein in its entirety.

Chimeric antigen receptors (CARs) are recombinant receptors containing an extracellular antigen recognition domain, a transmembrane domain, and a cytoplasmic signaling domain (such as CD3ζ, CD28, and 4-1BB). CARs possess both antigen-binding and T-cell-activating functions. Therefore, T cells expressing CARs can recognize a wide range of cell surface antigens, including glycolipids, carbohydrates, and proteins, and can attack malignant cells expressing these antigens through the activation of cytoplasmic costimulation. See Pang et al., 2018, Mol Cancer 17: 91, which is incorporated by reference herein in its entirety.

In some embodiments, the cell-based therapy is a Natural Killer (NK) cell-based therapy. NK cells are large, granular lymphocytes that have the ability to kill tumor cells without any prior sensitization or restriction of major histocompatibility complex (MHC) molecule expression. See Uppendahl et al., 2017, Frontiers in Immunology 8: 1825. Adoptive transfer of autologous lymphokine-activated killer (LAK) cells with high-dose IL-2 therapy have been evaluated in human clinical trials. Similar to LAK immunotherapy, cytokine-induced killer (CIK) cells arise from peripheral blood mononuclear cell cultures with stimulation of anti-CD3 mAb, IFN-γ, and IL-2. CIK cells are characterized by a mixed T-NK phenotype (CD3+CD56+) and demonstrate enhanced cytotoxic activity compared to LAK cells against ovarian and cervical cancer. Human clinical trials investigating adoptive transfer of autologous CIK cells following primary debulking surgery and adjuvant carboplatin/paclitaxel chemotherapy have also been conducted. See Liu et al., 2014, J Immunother 37(2): 116-122.

In some embodiments, the cell-based therapy is a dendritic cell-based immunotherapy. Vaccination with dendritic cells (DC)s treated with tumor lysates has been shown to increase therapeutic antitumor immune responses both in vitro and in vivo. See Jung et al., 2018, Translational Oncology 11(3): 686-690. DCs capture and process antigens, migrate into lymphoid organs, express lymphocyte costimulatory molecules, and secrete cytokines that initiate immune responses. They also stimulate immunological effector cells (T cells) that express receptors specific for tumor-associated antigens and reduce the number of immune repressors such as CD4+CD25+Foxp3+ regulatory T (Treg) cells. For example, a DC vaccination strategy for renal cell carcinoma (RCC), which is based on a tumor cell lysate-DC hybrid, showed therapeutic potential in preclinical and clinical trials. See Lim et al., 2007, Cancer Immunol Immunother 56: 1817-1829.

### 4. Cytokines

Several cytokines including IL-2, IL-12, IL-15, IL-18, and IL-21 have been used in the treatment of cancer for activation of immune cells such as NK cells and T cells. IL-2 was one of the first cytokines used clinically, with hopes of inducing antitumor immunity. As a single agent at high dose IL-2 induces remissions in some patients with renal cell carcinoma (RCC) and metastatic melanoma. Low dose IL-2 has also been investigated and aimed at selectively ligating the IL-2 αβγ receptor (IL-2Rαβγ) in an effort to reduce toxicity while maintaining biological activity. See Romee et al., 2014, Scientifica, Volume 2014, Article ID 205796, 18 pages, which is incorporated by reference herein in its entirety.

Interleukin-15 (IL-15) is a cytokine with structural similarity to Interleukin-2 (IL-2). Like IL-2, IL-15 binds to and signals through a complex composed of IL-2/IL-15 receptor beta chain (CD122) and the common gamma chain (gamma-C, CD132). Recombinant IL-15 has been evaluated for treatment of solid tumors (e.g. melanoma, renal cell carcinoma) and to support NK cells after adoptive transfer in cancer patients. See Romee et al., cited above.

IL-12 is a heterodimeric cytokine composed of p35 and p40 subunits (IL-12α and β chains), originally identified as “NK cell stimulatory factor (NKSF)” based on its ability to enhance NK cell cytotoxicity. Upon encounter with pathogens, IL-12 is released by activated dendritic cells and macrophages and binds to its cognate receptor, which is primarily expressed on activated T and NK cells. Numerous preclinical studies have suggested that IL-12 has antitumor potential. See Romee et al., cited above.

IL-18 is a member of the proinflammatory IL-1 family and, like IL-12, is secreted by activated phagocytes. IL-18 has demonstrated significant antitumor activity in preclinical animal models, and has been evaluated in human clinical trials. See Robertson et al., 2006, Clinical Cancer Research 12: 4265-4273.

IL-21 has been used for antitumor immunotherapy due to its ability to stimulate NK cells and CD8+ T cells. For ex vivo NK cell expansion, membrane bound IL-21 has been expressed in K562 stimulator cells, with effective results. See Denman et al., 2012, PLoS One 7(1)e30264. Recombinant human IL-21 was also shown to increase soluble CD25 and induce expression of perforin and granzyme B on CD8+ cells. IL-21 has been evaluated in several clinical trials for treatment of solid tumors. See Romee et al., cited above.

### 5. Cancer Vaccines

Therapeutic cancer vaccines eliminate cancer cells by strengthening a patients' own immune responses to the cancer, particularly CD8⁺ T cell mediated responses, with the assistance of suitable adjuvants. The therapeutic efficacy of cancer vaccines is dependent on the differential expression of tumor associated antigens (TAAs) by tumor cells relative to normal cells. TAAs derive from cellular proteins and should be mainly or selectively expressed on cancer cells to avoid either immune tolerance or autoimmunity effects. See Circelli et al., 2015, Vaccines 3(3): 544-555. Cancer vaccines include, for example, dendritic cell (DC) based vaccines, peptide/protein vaccines, genetic vaccines, and tumor cell vaccines. See Ye et al., 2018, J Cancer 9(2): 263-268.

The combination therapies of the present invention may be utilized for the treatment of oncological disorders. In some embodiments, the combination therapy of the virus engineered to comprise one or more polynucleotides that promote thanotransmission and the additional therapeutic agent inhibits tumor cell growth. Accordingly, the invention further provides methods of inhibiting tumor cell growth in a subject, comprising administering a virus engineered to comprise one or more polynucleotides that promote thanotransmission and at least one additional therapeutic agent to the subject, such that tumor cell growth is inhibited. In certain embodiments, treating cancer comprises extending survival or extending time to tumor progression as compared to a control. In some embodiments, the control is a subject that is treated with the additional therapeutic agent, but is not treated with the virus engineered to comprise one or more polynucleotides that promote thanotransmission. In some embodiments, the control is a subject that is treated with the virus engineered to comprise one or more polynucleotides that promote thanotransmission, but is not treated with the additional therapeutic agent. In some embodiments, the control is a subject that is not treated with the additional therapeutic agent or the virus engineered to comprise one or more polynucleotides that promote thanotransmission. In certain embodiments, the subject is a human subject. In some embodiments, the subject is identified as having a tumor prior to administration of the first dose of the virus engineered to comprise one or more polynucleotides that promote thanotransmission or the first dose of the additional therapeutic agent. In certain embodiments, the subject has a tumor at the time of the first administration of the virus engineered to comprise one or more polynucleotides that promote thanotransmission, or at the time of first administration of the additional therapeutic agent.

In certain embodiments, at least 1, 2, 3, 4, or 5 cycles of the combination therapy comprising the virus engineered to comprise one or more polynucleotides that promote thanotransmission and one or more additional therapeutic agents are administered to the subject. The subject is assessed for response criteria at the end of each cycle. The subject is also monitored throughout each cycle for adverse events (e.g., clotting, anemia, liver and kidney function, etc.) to ensure that the treatment regimen is being sufficiently tolerated.

It should be noted that more than one additional therapeutic agent, e.g., 2, 3, 4, 5, or more additional therapeutic agents, may be administered in combination with the virus engineered to comprise one or more polynucleotides that promote thanotransmission.

In one embodiment, administration of the virus engineered to comprise one or more polynucleotides that promote thanotransmission and the additional therapeutic agent as described herein results in one or more of, reducing tumor size, weight or volume, increasing time to progression, inhibiting tumor growth and/or prolonging the survival time of a subject having an oncological disorder. In certain embodiments, administration of the virus engineered to comprise one or more polynucleotides that promote thanotransmission and the additional therapeutic agent reduces tumor size, weight or volume, increases time to progression, inhibits tumor growth and/or prolongs the survival time of the subject by at least 1%, 2%, 3%, 4%, 5%, 10%, 20%, 30%, 40%, 50%, 60%, 70%, 80%, 90%, 100%, 200%, 300%, 400% or 500% relative to a corresponding control subject that is administered the virus engineered to comprise one or more polynucleotides that promote thanotransmission, but is not administered the additional therapeutic agent. In certain embodiments, administration of the virus engineered to comprise one or more polynucleotides that promote thanotransmission and the additional therapeutic agent reduces tumor size, weight or volume, increases time to progression, inhibits tumor growth and/or prolongs the survival time of a population of subjects afflicted with an oncological disorder by at least 1%, 2%, 3%, 4%, 5%, 10%, 20%, 30%, 40%, 50%, 60%, 70%, 80%, 90%, 100%, 200%, 300%, 400% or 500% relative to a corresponding population of control subjects afflicted with the oncological disorder that is administered the virus engineered to comprise one or more polynucleotides that promote thanotransmission, but is not administered the additional therapeutic agent. In other embodiments, administration of the virus engineered to comprise one or more polynucleotides that promote thanotransmission and the additional therapeutic agent stabilizes the oncological disorder in a subject with a progressive oncological disorder prior to treatment.

In certain embodiments, treatment with the virus engineered to comprise one or more polynucleotides that promote thanotransmission and the additional therapeutic agent (e.g. an immunotherapeutic) is combined with a further anti-neoplastic agent such as the standard of care for treatment of the particular cancer to be treated, for example by administering a standard dosage of one or more antineoplastic (e.g. chemotherapeutic) agents. The standard of care for a particular cancer type can be determined by one of skill in the art based on, for example, the type and severity of the cancer, the age, weight, gender, and/or medical history of the subject, and the success or failure of prior treatments. In certain embodiments of the invention, the standard of care includes any one of or a combination of surgery, radiation, hormone therapy, antibody therapy, therapy with growth factors, cytokines, and chemotherapy. In one embodiment, the additional anti-neoplastic agent is not an agent that induces iron-dependent cellular disassembly and/or an immune checkpoint modulator.

Additional anti-neoplastic agents suitable for use in the methods disclosed herein include, but are not limited to, chemotherapeutic agents (e.g., alkylating agents, such as Altretamine, Busulfan, Carboplatin, Carmustine, Chlorambucil, Cisplatin, Cyclophosphamide, Dacarbazine, Lomustine, Melphalan, Oxaliplatin, Temozolomide, Thiotepa; antimetabolites, such as 5-fluorouracil (5-FU), 6-mercaptopurine (6-MP); Capecitabine (Xeloda®), Cytarabine (Ara-C®), Floxuridine, Fludarabine, Gemcitabine (Gemzar®), Hydroxyurea, Methotrexate, Pemetrexed (Alimta®); anti-tumor antibiotics such as anthracyclines (e.g., Daunorubicin, Doxorubicin (Adriamycin®), Epirubicin, Idarubicin), Actinomycin-D, Bleomycin, Mitomycin-C, Mitoxantrone (also acts as a topoisomerase II inhibitor); topoisomerase inhibitors, such as Topotecan, Irinotecan (CPT-11), Etoposide (VP-16), Teniposide, Mitoxantrone (also acts as an anti-tumor antibiotic); mitotic inhibitors such as Docetaxel, Estramustine, Ixabepilone, Paclitaxel, Vinblastine, Vincristine, Vinorelbine; corticosteroids such as Prednisone, Methylprednisolone (Solumedrol®), Dexamethasone (Decadron®); enzymes such as L-asparaginase, and bortezomib (Velcade®)). Anti-neoplastic agents also include biologic anti-cancer agents, e.g., anti-TNF antibodies, e.g., adalimumab or infliximab; anti-CD20 antibodies, such as rituximab, anti-VEGF antibodies, such as bevacizumab; anti-HER2 antibodies, such as trastuzumab; anti-RSV, such as palivizumab.

### VIII. Pharmaceutical Compositions and Modes of Administration

In certain aspects, the present disclosure relates to a pharmaceutical composition comprising a virus engineered to comprise one or more polynucleotides that promote thanotransmission. The pharmaceutical compositions described herein may be administered to a subject in any suitable formulation. These include, for example, liquid, semi-solid, and solid dosage forms. The preferred form depends on the intended mode of administration and therapeutic application.

In certain embodiments the pharmaceutical composition is suitable for oral administration. In certain embodiments, the pharmaceutical composition is suitable for parenteral administration, including topical administration and intravenous, intraperitoneal, intramuscular, and subcutaneous, injections. In a particular embodiment, the pharmaceutical composition is suitable for intravenous administration. In a further particular embodiment, the pharmaceutical composition is suitable for intratumoral administration.

Pharmaceutical compositions for parenteral administration include aqueous solutions of the active compounds in water-soluble form. For intravenous administration, the formulation may be an aqueous solution. The aqueous solution may include Hank's solution, Ringer's solution, phosphate buffered saline (PBS), physiological saline buffer or other suitable salts or combinations to achieve the appropriate pH and osmolarity for parenterally delivered formulations. Aqueous solutions can be used to dilute the formulations for administration to the desired concentration. The aqueous solution may contain substances which increase the viscosity of the solution, such as sodium carboxymethyl cellulose, sorbitol, or dextran. In some embodiments, the formulation includes a phosphate buffer saline solution which contains sodium phosphate dibasic, potassium phosphate monobasic, potassium chloride, sodium chloride and water for injection.

Formulations suitable for topical administration include liquid or semi-liquid preparations suitable for penetration through the skin, such as liniments, lotions, creams, ointments or pastes, and drops suitable for administration to the eye, ear, or nose. Formulations suitable for oral administration include preparations containing an inert diluent or an assimilable edible carrier. The formulation for oral administration may be enclosed in hard or soft shell gelatin capsule, or it may be compressed into tablets, or it may be incorporated directly with the food of the diet. When the dosage unit form is a capsule, it may contain, in addition to materials of the above type, a liquid carrier. Various other materials may be present as coatings or to otherwise modify the physical form of the dosage unit. Pharmaceutical compositions suitable for use in the present invention include compositions wherein the active ingredients are contained in an effective amount to achieve its intended purpose. Determination of the effective amounts is well within the capability of those skilled in the art, especially in light of the detailed disclosure provided herein. In addition to the active ingredients, these pharmaceutical compositions may contain suitable pharmaceutically acceptable carriers including excipients and auxiliaries which facilitate processing of the active compounds into preparations which can be used pharmaceutically.

As will be readily apparent to one skilled in the art, the useful in vivo dosage to be administered and the particular mode of administration will vary depending upon the age, body weight, the severity of the affliction, and mammalian species treated, the particular compounds employed, and the specific use for which these compounds are employed. The determination of effective dosage levels, that is the dosage levels necessary to achieve the desired result, can be accomplished by one skilled in the art using routine methods, for example, human clinical trials, animal models, and in vitro studies.

In certain embodiments, the pharmaceutical composition is delivered orally. In certain embodiments, the composition is administered parenterally. In certain embodiments, the composition is delivered by injection or infusion. In certain embodiments, the composition is delivered topically including transmucosally. In certain embodiments, the composition is delivered by inhalation. In one embodiment, the compositions provided herein may be administered by injecting directly to a tumor. In some embodiments, the compositions may be administered by intravenous injection or intravenous infusion. In certain embodiments, administration is systemic. In certain embodiments, administration is local.

### EXAMPLES

This invention is further illustrated by the following examples which should not be construed as limiting. The contents of all references, GenBank Accession and Gene numbers, and published patents and patent applications cited throughout the application are hereby incorporated by reference. Those skilled in the art will recognize that the invention may be practiced with variations on the disclosed structures, materials, compositions and methods, and such variations are regarded as within the ambit of the invention.

### Example 1. Preparation of a Virus Containing One or More Heterologous Polynucleotides that Each Encodes a Polypeptide that Promotes Thanotransmission (e.g. RIPK3, ZBP1, MLKL and/or TRIF)

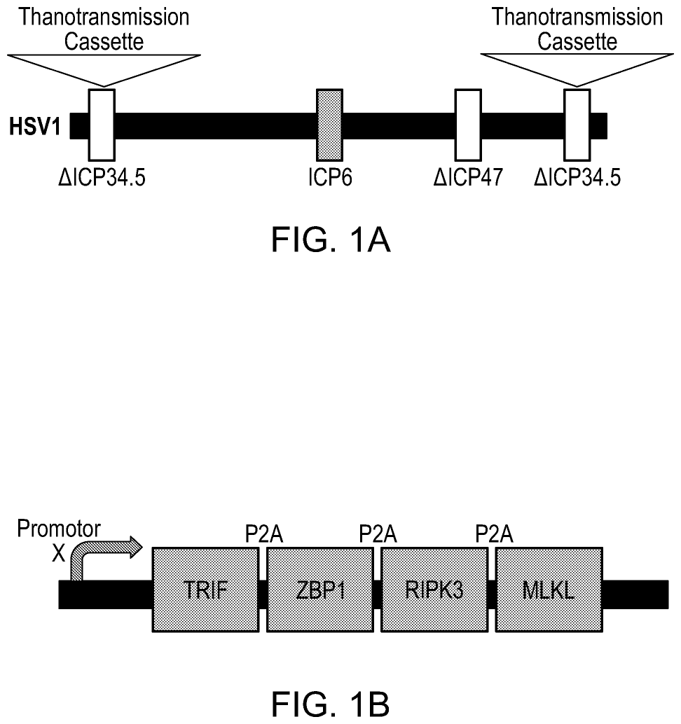

Shown in FIG. 1A is the architecture of a Thanotransmission Cassette (TC) and the locus of insertion to the viral genome. An example of a TC comprises genes encoding RIPK3, ZBP1, MLKL, and/or TRIF linked by P2A cleavage sites and expression driven by a viral promotor or cellular promotor (e.g. ICP34.5, CMV IE1, or EF1a) (FIG. 1B). Further examples include TCs comprising polynucleotides encoding TRIF, RIPK3, TRIF+RIPK3, TRIF+RIPK3+a caspase inhibitor (e.g., FADD-DN, vICA or cFLIP), or TRIF+RIPK3+a gasdermin (e.g., Gasdermin E). Particular examples of TCs that may be inserted into a viral genome are provided in Examples 9 to 14 below. The TC may be inserted into one or both of the ICP34.5 genes or alternatively into a neutral locus. Recombinant virus is generated by homologous recombination and then propogated in Vero cells. Viral stocks infect target cells at a range of multiplicity of infection (MOI) of 10-0.1, and infection is confirmed by evaluating expression of viral markers. Immunoblot or fluorescent tag analysis confirms the expression of the Thanotransmission Cassette. The virus may be, for example, HSV, Vaccinia, or an adenovirus.

### Example 2. Preparation of a Virus that Expresses a Polynucleotide (e.g. an siRNA or gRNA) that Reduces Expression of a Polypeptide that Regulates Thanotransmission

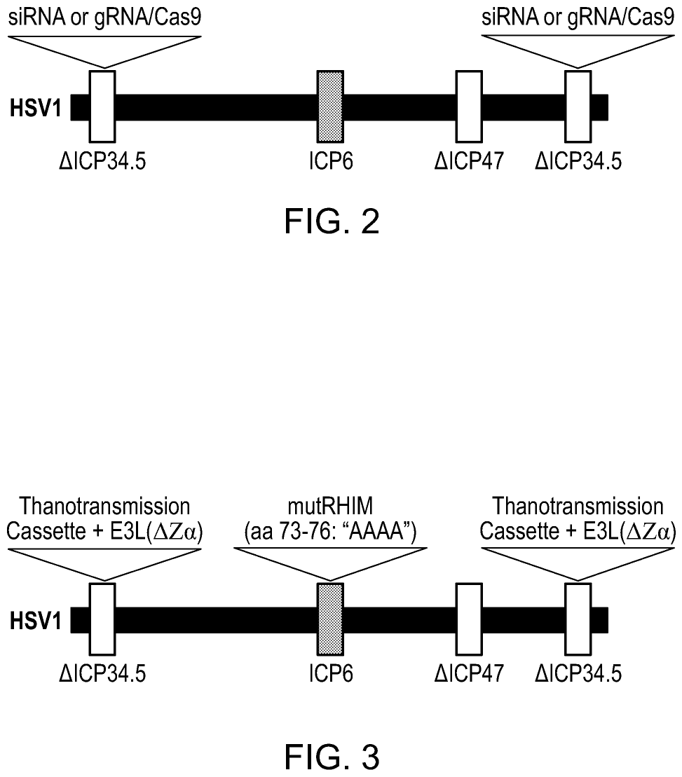

Shown in FIG. 2 is the architecture of a recombinant virus expressing a polynucleotide and detail of the locus of insertion to the viral genome. The locus of insertion may be in one or both ICP34.5 genes of the virus or alternatively at a neutral locus. Recombinant virus is generated by homologous recombination and then propogated in Vero cells. Viral stocks infect target cells at a range of MOI and infection is confirmed by evaluating expression of viral markers. Immunoblot or fluorescent tag analysis confirms the expression levels of the cellular proteins targeted by the virally encoded polynucleotide.

### Example 3. Preparation of a Virus Containing a Loss-of-Function Mutation in a Viral Gene that Prevents the Cell-Turnover Pathway Necroptosis

This example describes mutation of the ICP6 gene in HSV1 and mutation of the E3L gene in Vaccinia. Shown in FIG. 3 is the architecture of the mutant virus harboring mutations in the RHIM domain of HSV1-ICP6 and/or the Za domain of Vaccinia-E3L. Here, a mutant E3L(ΔZα) of Vaccinia is inserted to restore PKR inhibition but remain attenuated for replication within the CNS. Mutant virus is generated by homologous recombination and propogated in Vero cells. Viral stocks infect target cells at a range of MOI and infection is confirmed by evaluating expression of viral markers. Immunoblot analysis confirms expression of mutant ICP6 and E3L. For Vaccinia, the TC will be inserted in a neutral locus and the ZBP1 inhibitory Zα domain of E3L mutated.

### Example 4. Preparation of an Oncolytic Virus Comprising Mutations in Viral Genes and Polynucleotides Encoding Proteins that Promote Thanotransmission

This example describes mutation of ICP6 in HSV or mutation of E3L in Vaccinia in combination with a Thanotransmission Cassette containing one addition of a polynucleotide encoding RIPK3, ZBP1, MLKL, and TRIF. The mutations described in Examples 1-3 are combined. Mutant virus are generated by homologous recombination and propogated in Vero cells. A TC as described in FIG. 1 or Example 1 is cloned into a mutant viral backbone with ICP6 mutated, as described in FIG. 3. In other experiments, the polynucleotide cassette described in FIG. 2 is cloned into a mutant viral background as described in FIG. 3. Cloning is accomplished by homologous recombination and the viruses are propagated in Vero cells. Viruses are used to infect a human cell line (e.g. HEK 293), and expression of the TC is verified by immunoblot. Expression of the mutant viral proteins is verified by amplification of viral genomes and sequencing. Where the polynucleotide results in knockdown of a cellular gene, the expression levels of cellular gene targeted by siRNA/gRNA are evaluated.

### Example 5. Infection of Cancer Cells with Engineered Viruses Expressing Proteins that Promote Thanotransmission and Effects on Cell Turnover and Proliferation of the Cancer Cells

Multiple tumor cell lines (e.g. B16, CT26) are infected with the viruses from Examples 1-4, at varying MOIs. Productive infection is confirmed by quantifying an IE viral antigen. Growth curves of tumor cells at low (0.1) and high (10) MOI are used to evaluate the replicative capacity of viruses. The viability of infected tumor cells are measured by standard cell viability assays (e.g. cellular ATP content, LDH release, or cell imaging), to determine the susceptibility of tumor cells to virus-induced cell death. Tumor cells labeled with a cell permeable dye such as CFSE, are infected with viruses and the effect of infection on cell proliferation evaluated.

### Example 6. Evaluation of Cancer Cells Infected with Engineered Viruses Expressing Proteins that Promote Thanotransmission

Multiple tumor cell lines (e.g. B16, CT26) are infected by the parental and recombinant viruses described in Examples 1-4. Cell Turnover Factors (CTFs) released from infected cancer cells are evaluated for their ability to promote Thanotransmission in defined responder cell assays. Effects of CTFs are measured by reporter assays (e.g. NF-kB and/or IRF activity), and immunologic assays such as T cell proliferation, dendritic cell activation or macrophage differentiation. Mass spec analysis of CTF released from infected cancer cells identify the factors released from cells infected with oncolytic viruses.

### Example 7. Administration of Engineered Viruses Expressing Proteins that Promote Thanotransmission to Mouse Models of Cancer

WT BALB/c or C57B16/J mice are implanted with 4T1, CT26, B16 or MC38 tumors subcutaneously. Tumor cells are implanted at doses ranging from 1×10⁵ to 1×10⁶ per mouse. In some experiments, the mice are implanted at the orthotopic site, e.g., the mammary fat pad. When tumors become palpable, the mice are treated with intratumoral administration of engineered viruses as described herein, for example, the engineered viruses described in Examples 1-4. Viruses are administered at different dosing frequencies, ranging from once weekly, twice weekly or every 2 days. Virus doses range from 1×10⁶ pfu per mouse to 1×10⁸ pfu per mouse.

The growth of the tumor is measured three times a week. When the tumors reach a size of ˜1000 mm³, the tumors and draining lymph nodes (DLN) are harvested. The tumor immune response is characterized by quantifying the levels of immune cells in tumors and DLN by flow cytometry and the development of tumor-specific T cell responses evaluated by tetramer staining. The systemic immune response is measured by evaluating the ratio of activated cytotoxic T cells to helper T cells, as well as the levels of immunomodulatory cytokines in the plasma. In some studies, the tumors are harvested, and expression of the components of the Thanotransmission Module (e.g. the polypeptide encoded by the polynucleotide that promotes thanotransmission) or reduced expression of the siRNA/gRNA cellular targets are measured by immunoblot, immunofluorescence, and/or flow cytometry. In some experiments, the development of HSV-1⁺ immune response is monitored by ELISA for the appearance of virus-neutralizing antibodies, and compared to the development of an anti-tumor immune response.

In some experiments, mice will be inoculated with syngeneic bilateral subcutaneous tumors, and only one treated with virus. Virus levels and tumor-specific T cells responses are monitored in both treated and untreated tumors. In these experiments, tumor size of non treated tumors is measured to determine abscopal effect.

In some experiments, mice implanted with tumors are treated with intratumoral administration of recombinant viruses as described above, in combination with systemic administration of a checkpoint inhibitor. Anti-PD-1 or anti-CTLA-4 antibodies are administered intraperitoneally, at doses ranging from 1-10 mg/kg. Tumor growth kinetics and immune responses are measured as described above.

### Example 8. A Human Clinical Trial Investigating the Efficacy of an Engineered Virus to Treat a Cancer

A patient suffering from pancreatic cancer, lung cancer, brain cancer, bladder cancer, breast cancer, or head and neck cancer or colon cancer is treated using the compositions and methods disclosed herein. Mutant and recombinant HSV-1 based viral particles, based on the viruses described in Examples 1-4, are generated. Following plaque purification, virus stocks are further purified, buffer exchanged, and titered on Vero cells. For in vivo administration to a patient suffering from pancreatic cancer, lung cancer, or colon cancer, HSV particles are prepared in phosphate buffered solution (PBS) along with pharmaceutically acceptable stabilizing agents. On the day of treatment, 10⁷, 10⁸, 10⁹ or 10¹⁰ vector genomes in a volume of 1.0 mL with a pharmaceutically acceptable carrier are administered via intra-tumoral infusion. The patient is monitored for tumor regression using standard of care procedures at an appropriate time interval based on that patient's particular prognosis.

### Example 9. Induction of Cell Death in CT-26 Mouse Colon Carcinoma Cells Expressing One or More Thanotransmission Polypeptides

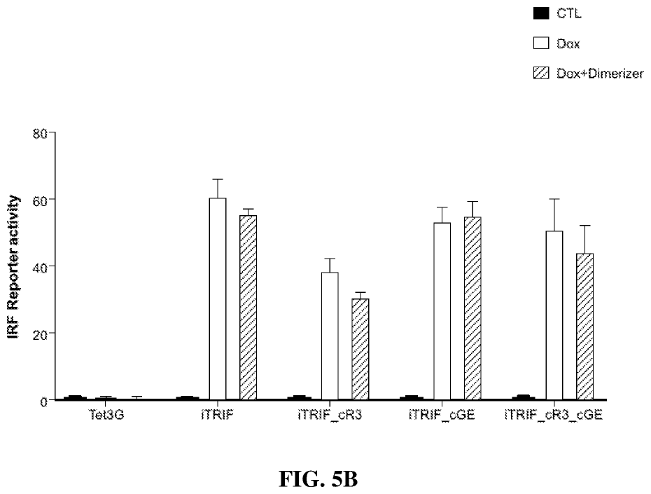

CT-26 mouse colon carcinoma cells (ATCC; CRL-2638) were transduced with lentivirus derived from the pLVX-Tet3G Vector (Takara; 631358) to establish stable Tet-On transactivator expression by the human PGK promotor. In the Tet-On system, gene expression is inducible by doxycycline. All lentiviral transductions were performed using standard production protocols utilizing 293T cells (ATCC; CRL-3216) and the Lentivirus Packaging Mix (Biosettia; pLV-PACK). CT-26-Tet3G cells were then transduced with the lentivirus expressing the human TRIF ORF (Accession No.: NM_182919) in pLVX-TRE3G (Takara; 631193). The CT-26-Tet3G cells were transduced alternatively, or in addition, with a vector expressing the mouse RIPK3 ORF (Accession No.: NM_019955.2); RIPK3 expression was driven by the constitutive PGK promotor derivative of pLV-EF1a-MCS-IRES-Hyg (Biosettia; cDNA-pLV02). Both ORFs were modified by the addition of two tandem DmrB domains that oligomerize upon binding to the B-B ligand (Takara; 635059), to allow for protein activation using the B/B homodimerizer (104) to promote oligomerization. After initial testing, dimerization using the B/B did not have a substantial effect on the activity of the TRIF construct, but did promote activity of the RIPK3 expressing construct. Therefore, in all subsequent experiments, B/B-induced dimerization was not employed to activate any constructs including TRIF, but was only employed to activate single constructs expressing RIPK3. As such, B/B dimerizer was included in the experimental setup, to ensure that experimental conditions were comparable across all groups, although it had no effect on TRIF-induced activity. For example, as shown in FIG. 5B and described in Example 10, addition of the dimerizer had little effect on IRF activity in macrophages treated with cell culture from the engineered CT-26 cells described above.

CT26 mouse colon carcinoma cells expressing the indicated thanotransmission modules were seeded and subsequently treated for 24 h with doxycycline (1 mg/mL; Sigma Aldrich, 0219895525) and B/B homodimerizer (1 μM) to promote expression and protein activation via oligomerization. Relative cell viability was determined at 24 h post-treatment using the RealTime-Glo MT Cell Viability Assay kit (Promega, Catalogue No. G9712) as per the manufacturer's instructions and graphed showing the relative viability measured by relative luminescence units (RLU).

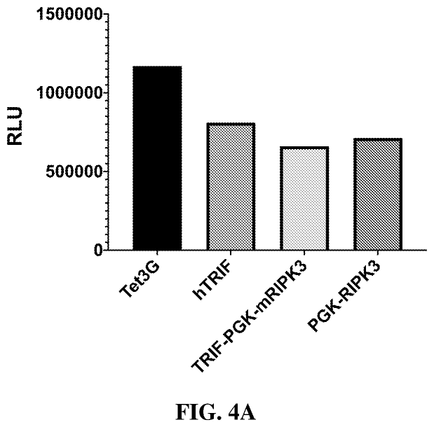

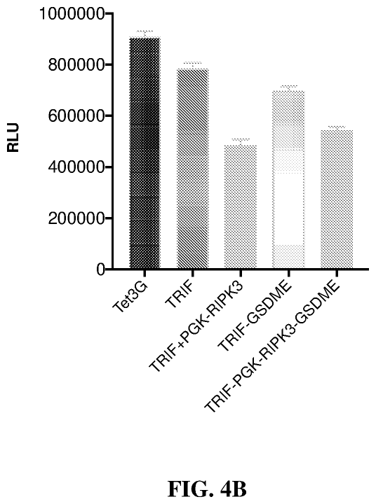

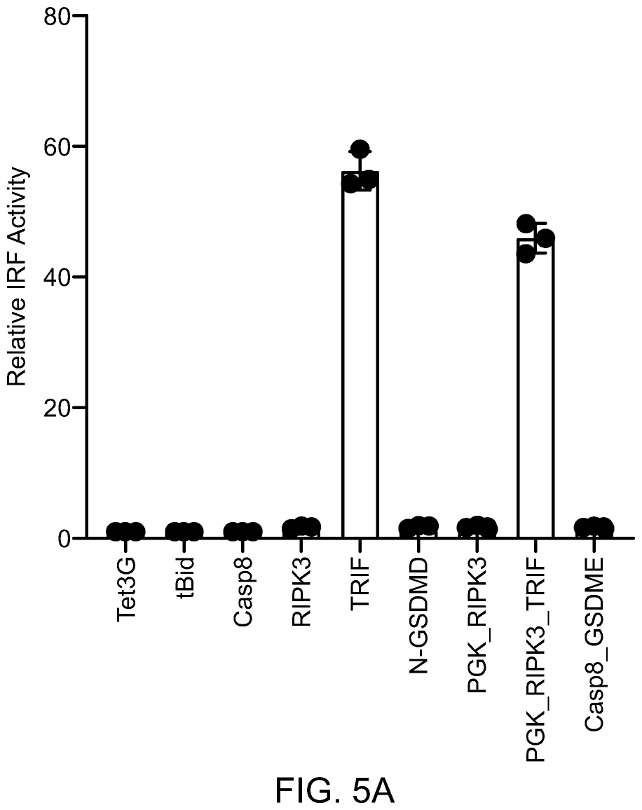

As shown in FIG. 4A, induced expression and oligomerization of TRIF, RIPK3, or TRIF+RIPK3 induced a reduction in cell viability relative to the CT-26-Tet3G (Tet3G) parental cell line. These results demonstrate that expression of one or more thanotransmission polypeptides in a cancer cell reduces viability of the cancer cell.

In a separate experiment, the effect of expression of Gasdermin E (GSDME) in cancer cells expressing TRIF, RIPK3, or TRIF and RIPK3 was examined. CT-26-Tet3G cells were transduced with human GSDME (NM_004403.3) cloned into the pLV-EF1a-MCS-IRES-Puro vector (Biosettia). GSDME was also transduced into the CT-26-Tet3G-TRIF and CT26-Tet3G-TRIF−RIPK3 cells described above. These cells were seeded and subsequently treated for 24 h with doxycycline (1 mg/mL; Sigma Aldrich, 0219895525) to promote expression. Relative cell viability was determined at 24 h post-treatment using the RealTime-Glo MT Cell Viability Assay kit (Promega, Catalogue No. G9712) as per the manufacturer's instructions and graphed showing the relative viability measured by relative luminescence units (RLU). The B/B dimerizer was not used for these experiments.

As shown in FIG. 4B, expression of TRIF, and TRIF+RIPK3 reduced cell viability relative to the CT-26-Tet3G parental cell line, confirming the results presented in FIG. 4A. Additionally, induction of TRIF or TRIF+RIPK3 protein expression in the GSDME-expressing cells also reduced cell viability compared to the CT-26-Tet3G parental cells. Together, these results demonstrate that expression of one or more thanotransmission polypeptides, including TRIF, RIPK3 and GSDME, in a cancer cell reduces viability of the cancer cell.

### Example 10. Effects of Cell Turnover Factors (CTFs) from CT-26 Mouse Colon Carcinoma Cells Expressing One or More Thanotransmission Polypeptides on Interferon Stimulated Gene (ISG) Reporters in Macrophages

J774-Dual™ cells (Invivogen, J774-NFIS) were seeded at 100,000 cells/well in a 96-well culture plate. J774-Dual™ cells were derived from the mouse J774.1 macrophage-like cell line by stable integration of two inducible reporter constructs. These cells express a secreted embryonic alkaline phosphatase (SEAP) reporter gene under the control of an IFN-β minimal promoter fused to five copies of an NF-κB transcriptional response element and three copies of the c-Rel binding site. J774-Dual™ cells also express the Lucia luciferase gene, which encodes a secreted luciferase, under the control of an ISG54 minimal promoter in conjunction with five interferon-stimulated response elements (ISREs). As a result, J774-Dual™ cells allow simultaneous study of the NF-κB pathway, by assessing the activity of SEAP, and the interferon regulatory factor (IRF) pathway, by monitoring the activity of Lucia luciferase.

Culture media containing cell turnover factors (CTFs) were generated from CT-26 mouse colon carcinoma cells as described in Example 9 above. In addition to the thanotransmission modules described in Example 9, an additional RIPK3 construct containing a fully Tet-inducible promoter was also evaluated. This Tet-inducible RIPK3 is designated as “RIPK3” in FIG. 5A, and the RIPK3 construct containing the PGK promoter (described in Example 9) is designated as “PGK_RIPK3” in FIG. 5A.

Controls were also included, that would be predicted to induce cell death, without immunostimulatory thanotransmission. These control constructs express i) the C-terminal caspase truncation of human Bid (NM_197966.3), ii) the N-terminal caspase truncation of human GSDMD (NM_001166237.1), iii) a synthetically dimerizable form of human caspase-8 (DmrB-caspase-8), or iv) both DmrB-caspase-8 and human GSDME (NM_004403.3). J774-Dual™ cells were then stimulated for 24 h with the indicated CTFs. Cell culture media were collected, and luciferase activity measured using the QUANTI-Luc (Invivogen; rep-qlc1) assay. Interferon-stimulated response element (ISRE) promotor activation was graphed relative to the control cell line, CT-26-Tet3G.

As shown in FIG. 5A, among the CT-26 cell lines examined, only culture media collected from cells that express TRIF (either alone or in combination with RIPK3) induced ISRE/IRF reporter gene activation in J774-Dual™ cells.

In a separate experiment, the effect of combined expression of Gasdermin E (GSDME) with TRIF or TRIF+RIPK3 was examined. Culture media containing CTFs were generated from the CT-26 cells expressing TRIF or TRIF+RIPK3 as described in Example 9, and in addition from CT-26 cells expressing TRIF+Gasdermin-E or TRIF+RIPK3+Gasdermin-E. As shown in FIG. 5B, culture media from CT-26 cells expressing TRIF (iTRIF), TRIF+RIPK3 (iTRIF_cR3), TRIF+Gasdermin-E (iTRIF_cGE), or TRIF+RIPK3+Gasdermin-E (iTRIF_cR3_cGE) each induced ISRE/IRF reporter gene activation in J774-Dual™ cells. As discussed in Example 9, addition of the dimerizer had little effect on ISRE/IRF reporter gene activation.

Taken together, these results demonstrate that CTFs produced from cancer cells expressing one or more thanotransmission polypeptides activate an immune-stimulatory pathway (i.e. the IRF pathway) in immune cells.

### Example 11. Effects of Cell Turnover Factors (CTFs) from CT-26 Mouse Colon Carcinoma Cells Expressing One or More Thanotransmission Polypeptides on Bone Marrow Derived Dendritic Cells (BMDCs)

Bone marrow cells were differentiated into dendritic cells for 8 days using GM-CSF sufficient RPMI culture medium. 400,000 cells per 2 mL were seeded in a 6-well plate. On day 8, bone marrow derived dendritic cells (BMDCs) were harvested and 100,000 cells/well were seeded in a 96-well plate. BMDCs were then stimulated with media containing CTFs derived from the engineered CT-26 cells described in Example 9. At 24 hours, stimulated cells were harvested and the expression of the cell surface markers CD86, CD40 and PD-L1 was measured by flow cytometry and the mean-fluorescent intensity (MFI) graphed relative to the Tet3G control. Sources of the antibodies were as follows: CD86 (Biolegend, Catalogue No. 105042); CD40 (Biolegend, Catalogue No. 102910); PD-L1 (Biolegend, Catalogue No. 124312). Expression of the cell surface markers CD86, CD40 and PD-L1 is indicative of dendritic cell maturation.

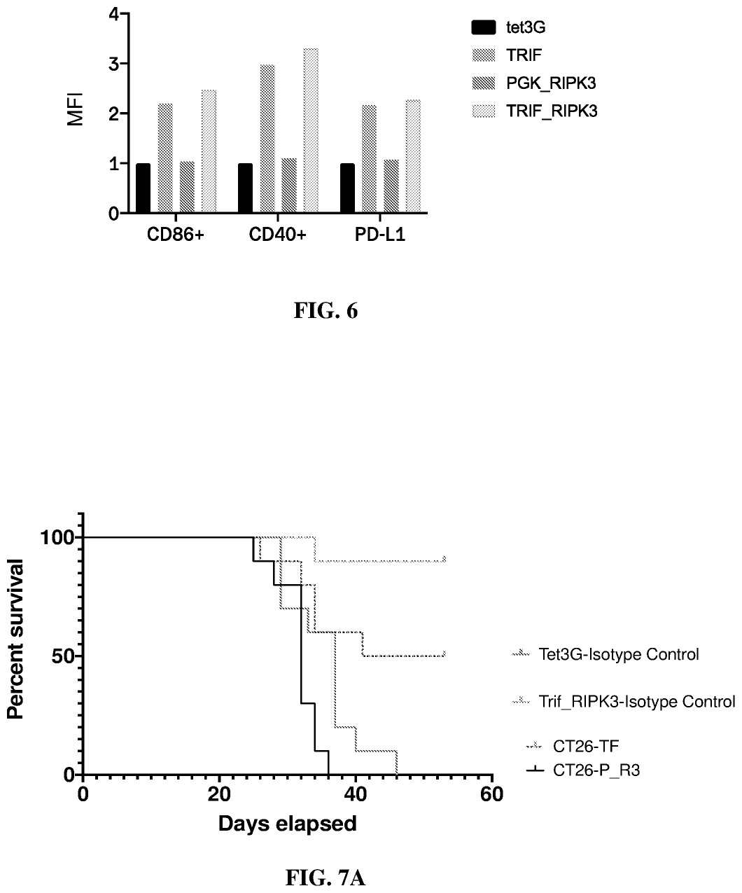

As show in FIG. 6, among the CT-26 cell lines examined, only culture media collected from cells engineered to express TRIF (either alone or in combination with RIPK3) elevated cell surface expression of CD86, CD40, or PD-L1. These results indicate that CTFs from CT-26 cells engineered to express TRIF or both TRIF and RIPK3 induced maturation of the dendritic cells. Upregulation of CD86 and CD40 in the dendritic cells indicates an increased ability to activate T cells. Therefore, the results indicate that CTFs from cancer cells engineered to express TRIF or TRIF and RIPK3 will induce maturation of dendritic cells and increase their ability to activate T cells.

### Example 12. Effect of Thanotransmission Polypeptide Expression Alone or in Combination with Anti-PD1 Antibody on Tumor Growth and Survival in a Mouse Model of Colon Carcinoma

CT-26 mouse colon carcinoma cells harboring the TRIF or TRIF+RIPK3 thanotransmission modules as described in Example 9 were trypsinized and resuspended in serum free media at 1×10⁶ cells/mL. Cells were injected (100 mL) into the right subcutaneous flank of BALB/c mice. From day 11 through day 18 post CT-26 cell injection, regular drinking water was supplemented with doxycycline (Sigma Aldrich, Catalogue No. D9891) at 2 mg/ml to induce thanotransmission polypeptide expression, and from day 11 through day 18, B/B homodimerizer (Takara, Catalogue No. 632622) 2 mg/kg was administered by daily IP injection. Anti-PD1 antibody (BioXcell, Catalogue No. BP0273) and isotype control were administered on day 14, day 17 and day 21. Mice were euthanized when the tumors reached 2000 mm³ in accordance with IACUC guidelines or at the experiment endpoint.

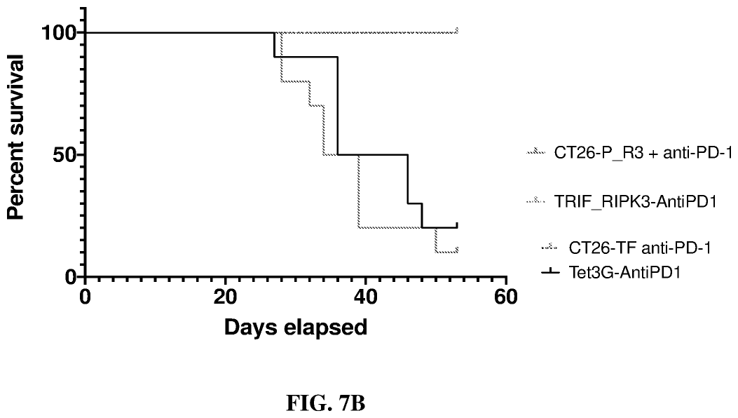

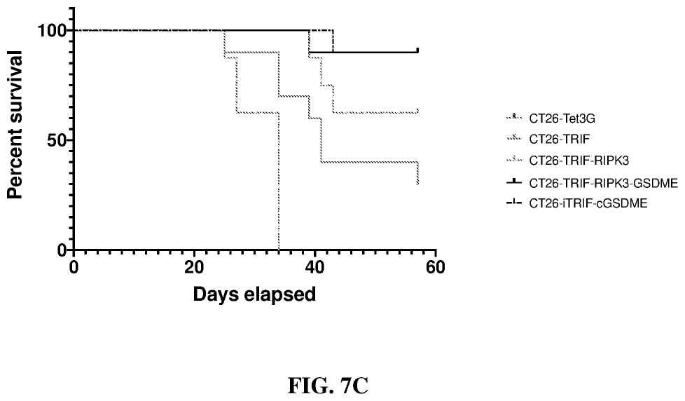

As shown in FIG. 7A, expression of TRIF alone (CT26-TF) increased survival as compared to the CT-26-Tet3G control (Tet3G-Isotype Control) and CT26-RIPK3 cells (CT26-P_R3), and an even greater benefit was observed with the combination of TRIF and RIPK3 (Trif_RIPK3-Isotype Control). As shown in FIG. 7B, the survival of mice injected with CT-26 cells harboring TRIF (CT26-TF) or CT-26 cells harboring TRIF+RIPK3 (TRIF_RIPK3) was enhanced by treatment with anti-PD-1 antibody, with both of these treatment groups exhibiting 100% survival (lines overlapping).

In a separate experiment, CT-26 mouse colon carcinoma cells harboring the TRIF+GSDME and TRIF+RIPK3+GSDME thanotransmission modules described in Example were trypsinized and resuspended in serum free media at 1×10⁶ cells/mL. No B/B homodimerizer was used for this experiment. Cells were injected (100 mL) into the right subcutaneous flank of BALB/c mice. From day 15 through day 21 post CT-26 cell injection, the mice were fed a Teklad base diet supplemented with 625 mg/kg of doxycycline hyclate (Envigo TD.01306). Mice were euthanized when the tumors reached 2000 mm³ in accordance with IACUC guidelines or at the experiment endpoint.

As shown in FIG. 7C, expression of GSDME in combination with TRIF or TRIF+RIPK3 further enhanced survival relative to mice implanted with tumors expressing TRIF alone or TRIF−RIPK3 alone.

### Example 13. Effects of Chemical Caspase Inhibitors on U937 Human Myeloid Leukemia Cells Expressing Thanotransmission Polypeptides

U937 human myeloid leukemia cells and THP1-Dual cells were acquired from ATCC and Invivogen respectively. U937 is a myeloid leukemia cell line. U937 cells expressing human thanotransmission polypeptides (tBid, Caspase 8, RIPK3 or TRIF) were generated using the methods described in Examples 9 and 10, and the doxycycline-inducible expression system described in Example 9.

THP1-Dual cells are a human monocytic cell line that induces reporter proteins upon activation of either NF-kB or IRF pathways. It expresses a secreted embryonic alkaline phosphatase (SEAP) reporter gene driven by an IFN-β minimal promoter fused to five copies of the NF-κB consensus transcriptional response element and three copies of the c-Rel binding site. THP1-Dual cells also feature the Lucia gene, a secreted luciferase reporter gene, under the control of an ISG54 minimal promoter in conjunction with five IFN-stimulated response elements. As a result, THP1-Dual cells allow the simultaneous study of the NF-kB pathway, by monitoring the activity of SEAP, and the IRF pathway, by assessing the activity of a secreted luciferase (Lucia).

To generate conditioned media, 5 million U937-tet3G, U937-tBid, U937-caspase8, U937-RIPK3 or U937-TRIF cells were seeded in a 10 cm dish in RPMI, and subsequently treated for 24 h with doxycycline (1 μg/mL) to induce expression. B/B homodimerizer (100 nM) was added to U937-caspase8, U937-RIPK3 and U937-TRIF cell cultures to promote expression and protein activation via oligomerization. Furthermore, U937-TRIF cells were additionally treated with 4 μM Q-VD-Oph (pan-caspase inhibitor), 10 μM GSK872 (RIPK3 inhibitor) or the combination of both. After cells were incubated for 24 hours, the conditioned media were harvested and sterile filtered.

To measure the thanotransmission polypeptide effect on NF-kB or IRF reporter expression, 100,000 THP1-Dual cells/well were seeded in a 96-well flat-bottom plate in 100 μl volume. 100 μl of conditioned media that generated from U937 cells expressing thanotransmission modules were added to each well. After 24 hour incubation period, 20 μl of THP1-Dual cell culture supernatants were transferred to a flat-bottom 96-well white (opaque) assay plate, and 50 μl of QUANTI-Luc assay solution was added to each well immediately prior to reading luminescence by a plate reader. To measure NF-kB activity, 20 μl of THP1-Dual culture supernatants were transferred to a flat-bottom 96-well clear assay plate, and 180 μl of resuspended QUANTI-Blue solution was added to each well. The plate was incubated at 37° C. for 1 hour and SEAP levels were then measured using a plate reader at 655 nm.

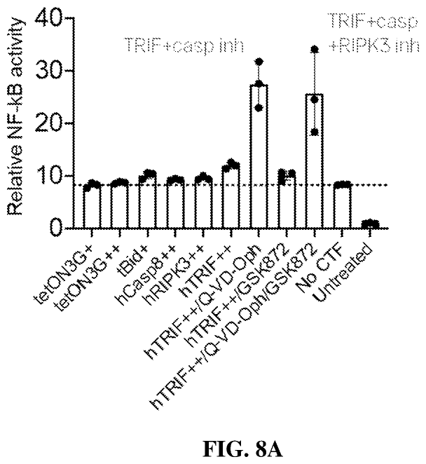

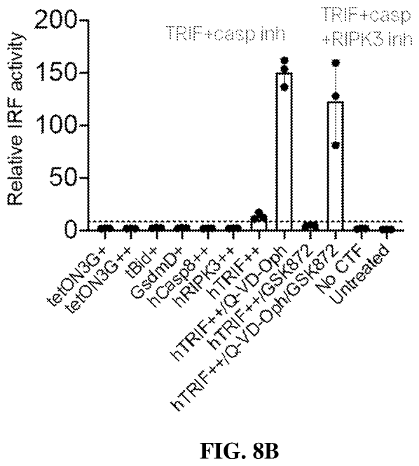

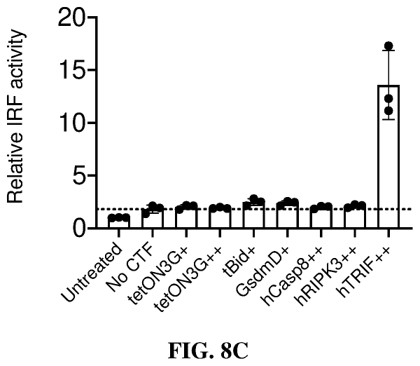

As shown in FIGS. 8A and 8B, treatment of THP-1 Dual cells with cell culture from U937-TRIF cells treated with caspase inhibitor (Q-VD-Oph) alone or in combination with RIPK3 inhibitor (Q-VD-Oph+GSK872) greatly increased NF-kB activation and IRF activity. (In FIGS. 8A-8C, + indicates U937 cells treated with doxycycline, and ++ indicates U937 cells treated with doxycycline and B/B homodimerizer). Cell culture media from U937-TRIF cells treated with RIPK3 inhibitor alone had little effect on NF-kB activation of the THP-1 Dual cells, indicating that the increased NF-kB activation was due to caspase inhibition. As shown in FIGS. 8B and 8C, treatment of THP-1 Dual cells with cell culture media from U937-TRIF cells that were not treated with caspase inhibitor also increased IRF activity, although to a lesser extent than U937-TRIF cells treated with caspase inhibitor.

Taken together, these results demonstrate that CTFs produced from human cancer cells expressing TRIF activate immune-stimulatory pathways (i.e. the NF-kB and IRF pathways) in immune cells, and that caspase inhibition enhances this effect.

### Example 14. Modulation of Thanotransmission in CT-26 Mouse Colon Carcinoma Cells by Expressing Combinatorial Thanotransmission Polypeptides Including Caspase Inhibitor Proteins

The experiment described in this example tested the effect of expression of caspase inhibitor proteins on thanotransmission in cancer cells expressing TRIF and RIPK3.

CT26 mouse colon carcinoma cells expressing the thanotransmission polypeptides TRIF and RIPK3, as described in Example 9, were transduced with genes encoding: (i) a dominant negative version of human Fas-associated protein with death domain (FADD; Accession No. NM_003824); (ii) the short version of human cellular FLICE-like inhibitory protein (cFLIPs; Accession No. NM_001127184.4); or (iii) viral inhibitor of Caspase (vICA, HCMV gene UL36; Accession No. NC_006273.2) in order to modulate thanotransmission by inhibiting caspase activity. FADD-DN, cFLIPs and vICA were each cloned into the pLV-EF1a-MCS-IRES-Puro vector (Biosettia), and used to transduce CT26-TRIF−RIPK3 expressing cells.

These cells were seeded and subsequently treated for 24 h with doxycycline (1 mg/mL; Sigma Aldrich, 0219895525) to promote expression. B/B homodimerizer was not used in this experiment. Relative cell viability was determined at 24 h post-treatment using the RealTime-Glo MT Cell Viability Assay kit (Promega, Catalogue No. G9712) as per the manufacturer's instructions and graphed showing the relative viability measured by relative luminescence units (RLU).

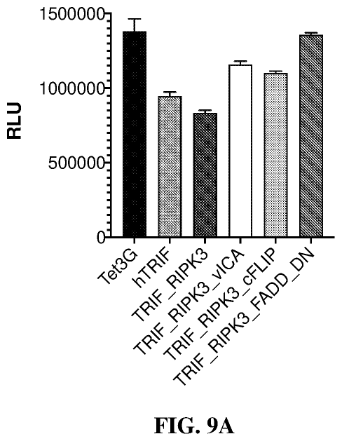

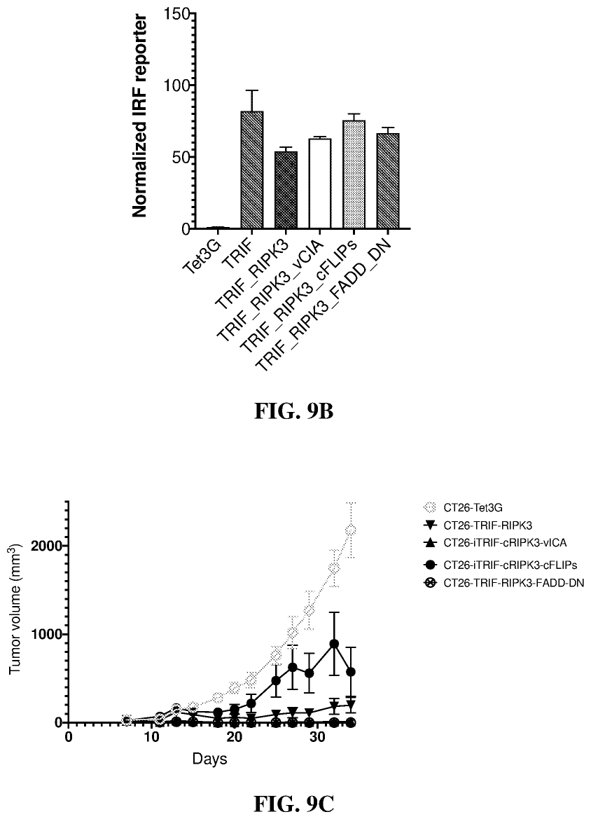

As shown in FIG. 9A, expression of any one of FADD-DN, cFL1Ps or vICA in the CT26-TRIF+RIPK3 cells attenuated the decrease in cancer cell viability induced by TRIF+RIPK3 expression. However, expression of cFLIPs+TRIF+RIPK3 or vICA+TRIF+RIPK3 in CT26 cells still reduced cancer cell viability relative to the parental line CT26-Tet3G cell line, just to a lesser extent than TRIF−RIPK3 alone. See FIG. 9A.

Next, culture media containing CTFs were generated from CT-26 mouse colon carcinoma cells as described above. J774-Dual™ cells were then stimulated for 24 h with the indicated CTFs. Cell culture media were collected, and luciferase activity measured using the QUANTI-Luc (Invivogen; rep-qlc1) assay. Interferon-stimulated response element (ISRE) promotor activation was graphed relative to the control cell line, Tet3G. As shown in FIG. 9B, media collected from CT26 cell lines expressing TRIF or TRIF+RIPK3 induced IRF reporter expression in J774-Dual cells. In addition, media from CT26 cells expressing FADD-DN, cFLIPs or vICA in addition to TRIF+RIPK3 also induced IRF reporter activation in J774-Dual cells.

CT-26-TRIF+RIPK3 mouse colon carcinoma cells harboring the FADD-DN, cFLIPs or vICA thanotransmission modules described above were trypsinized and resuspended in serum free media at 1×10⁶ cells/mL. No B/B homodimerizer was used in this experiment. Cells were injected (100 μL) into the right subcutaneous flank of immune-competent BALB/c mice. From day 15 through day 21 post CT-26 cell injection, the mice were fed a Teklad base diet supplemented with 625 mg/kg of doxycycline hyclate (Envigo TD.01306). Mice were euthanized when the tumors reached 2000 mm³ in accordance with IACUC guidelines or at the experiment endpoint.

As shown in FIG. 9C, growth of all tumors expressing a thanotransmission module (i.e. TRIF+RIPK3, TRIF+RIPK3+FADD-DN, TRIF+RIPK3+cFLIPS, or TRIF+RIPK3+vICA) was reduced relative to control CT26-Tet3G cells. In particular, expression of FADD-DN or vICA in combination with TRIF+RIPK3 further reduced tumor growth, as compared to the parental CT26-TRIF+RIPK3 cells. Interestingly, although the thanotransmission modules comprising FADD-DN or vICA in addition to TRIF+RIPK3 were most effective in reducing tumor growth in vivo, the FADD-DN+TRIF+RIPK3 had little effect on CT26 cancer cell viability in vitro relative to the TRIF+RIPK3 cells, while vICA+TRIF+RIPK3 coexpression enhanced cell killing in vitro relative to TRIF+RIPK3. These results suggest that in addition to the magnitude of cancer cell killing by thanotransmission modules, the precise cell turnover factor (CTF) profile produced by the cancer cells due to expression of these modules may also contribute to the immune response to the tumor cells in vivo.

1. A virus engineered to comprise one or more polynucleotides that promote thanotransmission by a target cell.

2. The virus of claim 1, wherein at least one of the polynucleotides is heterologous to the virus.

3. The virus of claim 1 or 2, wherein at least one of the polynucleotides is heterologous to the target cell.

4. The virus of any one of claims 1 to 3, wherein at least one of the polynucleotides promotes thanotransmission by the target cell by increasing expression or activity in the target cell of a thanotransmission polypeptide.

5. The virus of any one of claims 1 to 4, wherein at least one of the polynucleotides encodes a thanotransmission polypeptide.

6. The virus of any one of claims 1 to 5, wherein at least one of the polynucleotides promotes thanotransmission by the target cell by reducing expression or activity in the target cell of a polypeptide that suppresses thanotransmission.

7. The virus of any one of claims 1 to 6, wherein at least one of the polynucleotides encodes an RNA molecule that reduces expression or activity in the target cell of a polypeptide that suppresses thanotransmission.

8. The virus of any one of claims 1 to 7, wherein expression of at least one of the polynucleotides in the target cell alters a cell turnover pathway in the target cell.

9. The virus of any one of claims 1 to 8, wherein at least one of the polynucleotides encodes a wild type protein or functional fragment thereof.

10. The virus of any one of claims 1 to 9, wherein at least one of the polynucleotides encodes a death fold domain.

11. The virus of claim 10, wherein the death fold domain is selected from the group consisting of a death domain, a pyrin domain, a Death Effector Domain (DED), a C-terminal caspase recruitment domain (CARD), and variants thereof.

12. The virus of claim 11, wherein the death domain is from a protein selected from the group consisting of Fas-associated protein with death domain (FADD), Fas, Tumor necrosis factor receptor type 1 associated death domain (TRADD), Tumor necrosis factor receptor type 1 (TNFR1), and variants thereof.

13. The virus of claim 11, wherein the pyrin domain is from a protein selected from the group consisting of NLR Family Pyrin Domain Containing 3 (NLRP3) and apoptosis-associated speck-like protein (ASC).

14. The virus of claim 11, wherein the Death Effector Domain (DED) is from a protein selected from the group consisting of Fas-associated protein with death domain (FADD), caspase-8 and caspase-10.

15. The virus of claim 11, wherein the CARD is from a protein selected from the group consisting of RIP-associated ICH1/CED3-homologous protein (RAIDD), apoptosis-associated speck-like protein (ASC), mitochondrial antiviral-signaling protein (MAVS), caspase-1, and variants thereof.

16. The virus of any one of claims 1 to 15, wherein at least one of the polynucleotides encodes a Toll/interleukin-1 receptor (TIR) domain.

17. The virus of claim 16, wherein the TIR domain is from a protein selected from the group consisting of Myeloid Differentiation Primary Response Protein 88 (MyD88), Toll/interleukin-1 receptor (TIR)-domain-containing adapter-inducing interferon-β (TRIF), Toll Like Receptor 3 (TLR3), Toll Like Receptor 4 (TLR4), TIR Domain Containing Adaptor Protein (TIRAP), and Translocating chain-associated membrane protein (TRAM)

18. The virus of any one of claims 1 to 17, wherein the one or more polynucleotides encode any one or more of receptor-interacting serine/threonine-protein kinase 3 (RIPK3), Z-DNA-binding protein 1 (ZBP1), mixed lineage kinase domain like pseudokinase (MLKL), Toll/interleukin-1 receptor (TIR)-domain-containing adapter-inducing interferon-β (TRIF), an N-terminal truncation of TRIF that comprises only a TIR domain and a RHIM domain, Interferon Regulatory Factor 3 (IRF3), Fas-associated protein with death domain (FADD), a truncated FADD, Tumor necrosis factor receptor type 1 associated death domain (TRADD), and Cellular FLICE (FADD-like IL-1β-converting enzyme)-inhibitory protein (c-FLIP).

19. The virus of claim 18, wherein the polynucleotide encoding ZBP1 comprises a deletion of receptor-interacting protein homotypic interaction motif (RHIM) C, a deletion of RHIM D, and a deletion at the N-terminus of a Zα1 domain.

20. The virus of any one of claims 1 to 19, wherein at least one of the polynucleotides inhibits expression or activity of receptor-interacting serine/threonine-protein kinase 1 (RIPK1).

21. The virus of any one of claims 1 to 20, wherein at least one of the polynucleotides encodes a fusogenic protein.

22. The virus of claim 21, wherein the fusogenic protein is glycoprotein from gibbon ape leukemia virus (GALV) and has the R transmembrane peptide mutated or removed (GALV-R-).

23. The virus of any one of claims 1 to 22, wherein at least one of the polynucleotides encodes an immune stimulatory protein.

24. The virus of claim 23, wherein the immune stimulatory protein is an antagonist of transforming growth factor beta (TGF-β), a colony-stimulating factor, a cytokine, or an immune checkpoint modulator.

25. The virus of claim 24, wherein the colony-stimulating factor is granulocyte-macrophage colony-stimulating factor (GM-CSF).

26. The virus of claim 25, wherein the polynucleotide encoding GM-CSF is inserted into the ICP34.5 gene locus of the virus.

27. The virus of claim 24, wherein the cytokine is an interleukin.

28. The virus of claim 27, wherein the interleukin is selected from the group consisting of IL-1α, IL-1β, IL-2, IL-4, IL-12, IL-15, IL-18, IL-21, IL-24, IL-33, IL-36a, IL-36β and IL-36γ.

29. The virus of claim 24, wherein the cytokine is selected from the group consisting of a type I interferon, interferon gamma, a type III interferon and TNF alpha.

30. The virus of claim 24, wherein the immune checkpoint modulator is an antagonist of an inhibitory immune checkpoint protein.

31. The virus of claim 30, wherein the inhibitory immune checkpoint protein is selected from the group consisting of ADORA2A, B7-H3, B7-H4, IDO, KIR, VISTA, PD-1, PD-L1, PD-L2, LAGS, Tim3, BTLA and CTLA4.

32. The virus of claim 24, wherein the immune checkpoint modulator is an agonist of a stimulatory immune checkpoint protein.

33. The virus of claim 32, wherein the stimulatory immune checkpoint protein is selected from the group consisting of CD27, CD28, CD40, CD122, OX40, GITR, ICOS and 4-1BB.

34. The virus of claim 32, wherein the agonist of the stimulatory immune checkpoint protein is selected from CD40 ligand (CD40L), ICOS ligand, GITR ligand, 4-1-BB ligand, 0X40 Ligand and a modified version of any thereof.

35. The virus of claim 32, wherein the agonist of the stimulatory immune checkpoint protein is an antibody agonist of a protein selected from CD40, ICOS, GITR, 4-1-BB and 0X40.

36. The virus of claim 23, wherein the immune stimulatory protein is an flt3 ligand or an antibody agonist of flt3.

37. The virus of any one of claims 1 to 36, wherein at least one of the polynucleotides is a suicide gene.

38. The virus of claim 37, wherein the suicide gene encodes a polypeptide selected from the group consisting of FK506 binding protein (FKBP)-FAS, FKBP-caspase-8, FKBP-caspase-9, a polypeptide having cytosine deaminase (CDase) activity, a polypeptide having thymidine kinase activity, a polypeptide having uracil phosphoribosyl transferase (UPRTase) activity, and a polypeptide having purine nucleoside phosphorylase activity.

39. The virus of claim 38, wherein the polypeptide having CDase activity is FCY1, FCA1 or CodA.

40. The virus of claim 38, wherein the polypeptide having UPRTase activity is FUR1 or a variant thereof.

41. The virus of claim 40, wherein the variant of FUR1 is FUR1Δ105.

42. The virus of claim 37, wherein the suicide gene encodes a chimeric protein having CDase and UPRTase activity.

43. The virus of claim 42, wherein the chimeric protein is selected from the group consisting of codA::upp, FCY1::FUR1, FCY1::FUR1Δ105 (FCU1) and FCU1-8 polypeptides.

44. The virus of any one of claims 1 to 43, wherein at least one of the polynucleotides encodes a polypeptide selected from the group consisting of gasdermin-A (GSDM-A), gasdermin-B (GSDM-B), gasdermin-C (GSDM-C), gasdermin-D (GSDM-D), gasdermin-E (GSDM-E), apoptosis-associated speck-like protein containing C-terminal caspase recruitment domain (ASC-CARD) with a dimerization domain, and mutants thereof.

45. The virus of any one of claims 1 to 44, wherein the one or more polynucleotides that promote thanotransmision encode two or more different thanotransmission polypeptides, wherein the two or more thanotransmission polypeptides are selected from the group consisting of TRADD, TRAF2, TRAF6, cIAP1, cIAP2, XIAP, NOD2, MyD88, TRAM, HOIL, HOIP, Sharpin, IKKg, IKKa, IKKb, RelA, MAVS, RIGI, MDA5, Tak1, TBK1, IKKe, IRF3, IRF7, IRF1, TRAF3, a Caspase, FADD, TNFR1, TRAILR1, TRAILR2, FAS, Bax, Bak, Bim, Bid, Noxa, Puma, TRIF, ZBP1, RIPK1, RIPK3, MLKL, Gasdermin A, Gasdermin B, Gasdermin C, Gasdermin D, Gasdermin E, a tumor necrosis factor receptor superfamily (TNFSF) protein, variants thereof, and functional fragments thereof.

46. The virus of claim 45, wherein at least one of the polynucleotides encodes a chimeric protein comprising at least two of the thanotransmission polypeptides.

47. The virus of claim 45, wherein at least one of the polynucleotides is transcribed as a single transcript that encodes the two or more different thanotransmission polypeptides.

48. The virus of any one of claims 45 to 47, wherein at least two of the thanotransmission polypeptides encoded by the one or more polynucleotides activate NF-kB.

49. The virus of any one of claims 45 to 47, wherein at least two of the thanotransmission polypeptides encoded by the one or more polynucleotides activate IRF3 and/or IRF7.

50. The virus of any one of claims 45 to 47, wherein at least two of the thanotransmission polypeptides encoded by the one or more polynucleotides promote extrinsic apoptosis.

51. The virus of any one of claims 45 to 47, wherein at least two of the thanotransmission polypeptides encoded by the one or more polynucleotides promote programmed necrosis.

52. The virus of any one of claims 45 to 47, wherein at least one of the thanotransmission polypeptides encoded by the one or more thanotransmission polynucleotides activates NF-kB, and at least one of the thanotransmission polypeptides encoded by the one or more polynucleotides activates IRF3 and/or IRF7.

53. The virus of any one of claims 45 to 47, wherein at least one of the thanotransmission polypeptides encoded by the one or more polynucleotides activates NF-kB, and at least one of the thanotransmission polypeptides encoded by the one or more polynucleotides promotes extrinsic apoptosis.

54. The virus of any one of claims 45 to 47, wherein at least one of the thanotransmission polypeptides encoded by the one or more polynucleotides activates NF-kB, and at least one of the thanotransmission polypeptides encoded by the one or more polynucleotides promotes programmed necrosis.

55. The virus of any one of claims 45 to 47, wherein at least one of the thanotransmission polypeptides encoded by the one or more polynucleotides activates IRF3 and/or IRF7, and at least one of the thanotransmission polypeptides encoded by the one or more polynucleotides promotes extrinsic apoptosis.

56. The virus of any one of claims 45 to 47, wherein at least one of the thanotransmission polypeptides encoded by the one or more polynucleotides activates IRF3 and/or IRF7, and at least one of the thanotransmission polypeptides encoded by the one or more polynucleotides promotes programmed necrosis.

57. The virus of any one of claims 45 to 47, wherein at least one of the thanotransmission polypeptides encoded by the one or more polynucleotides promotes extrinsic apoptosis, and at least one of the thanotransmission polypeptides encoded by the one or more thanotransmission polynucleotides promotes programmed necrosis.

58. The virus of any one of claims 51, 54, 56 and 57, wherein the programmed necrosis comprises necroptosis.

59. The virus of any one of claims 51, 54, 56 and 57, wherein the programmed necrosis comprises pyroptosis.

60. The virus of any one of claims 48 and 52 to 54, wherein the thanotransmission polypeptide that activates NF-kB is selected from the group consisting of TRIF, TRADD, TRAF2, TRAF6, cIAP1, cIAP2, XIAP, NOD2, MyD88, TRAM, HOIL, HOIP, Sharpin, IKKg, IKKa, IKKb, RelA, MAVS, RIGI, MDA5, Tak1, a TNFSF protein, and functional fragments thereof.

61. The virus of any one of claims 49, 52, 55 and 56, wherein the thanotransmission polypeptide that activates IRF3 and/or IRF7 is selected from the group consisting of TRIF, MyD88, MAVS, TBK1, IKKe, IRF3, IRF7, IRF1, TRAF3 and functional fragments thereof.

62. The virus of any one of claims 50, 53, 55 and 57, wherein the thanotransmission polypeptide that promotes extrinsic apoptosis is selected from the group consisting of TRIF, RIPK1, Caspase, FADD, TRADD, TNFR1, TRAILR1, TRAILR2, FAS, Bax, Bak, Bim, Bid, Noxa, Puma, and functional fragments thereof.

63. The virus of any one of claims 51, 54, 56 and 57, wherein the thanotransmission polypeptide that promotes programmed necrosis is selected from the group consisting of TRIF, ZBP1, RIPK1, RIPK3, MLKL, a Gasdermin, and functional fragments thereof.

64. The virus of any one of claims 1 to 63, wherein, at least one of the thanotransmission polypeptides encoded by the one or more polynucleotides comprises TRIF or a functional fragment thereof.

65. The virus of any one of claims 1 to 63, wherein at least one of the thanotransmission polypeptides encoded by the one or more polynucleotides comprises RIPK3 or a functional fragment thereof.

66. The virus of any one of claims 1 to 63, wherein at least one of the thanotransmission polypeptides encoded by the one or more polynucleotides comprises TRIF or a functional fragment thereof, and at least one of the thanotransmission polypeptides encoded by the one or more polynucleotides comprises RIPK3 or a functional fragment thereof.

67. The virus of any one of claims 1 to 63, wherein at least one of the thanotransmission polypeptides encoded by the one or more polynucleotides comprises MAVS or a functional fragment thereof, and at least one of the thanotransmission polypeptides encoded by the one or more polynucleotides comprises RIPK3 or a functional fragment thereof.

68. The virus of any one of claims 1 to 67, wherein the one or more polynucleotides further encode a polypeptide that inhibits caspase activity.

69. The virus of claim 68, wherein the polypeptide that inhibits caspase activity is selected from the group consisting of a FADD dominant negative mutant (FADD-DN), cFLIP, vICA, a caspase 8 dominant negative mutant (Casp8-DN), cIAP1, cIAP2, Tak1, an IKK, and functional fragments thereof.

70. The virus of claim 68, wherein the polypeptide that inhibits caspase activity is FADD-DN.

71. The virus of claim 68, wherein the polypeptide that inhibits caspase activity is cFLIP.

72. The virus of claim 68, wherein the polypeptide that inhibits caspase activity is vICA.

73. The virus of any one of claims 1 to 72, wherein the one or more polynucleotides encode at least one Gasdermin or a functional fragment thereof.

74. The virus of claim 73, wherein at least one of the thanotransmission polypeptides comprises TRIF or a functional fragment thereof, at least one of the thanotransmission polypeptides comprises RIPK3 or a functional fragment thereof, and at least one of the thanotransmission polypeptides comprises a Gasdermin or a functional fragment thereof.

75. The virus of claim 73, wherein at least one of the thanotransmission polypeptides comprises MAVS or a functional fragment thereof, at least one of the thanotransmission polypeptides comprises RIPK3 or a functional fragment thereof, and at least one of the thanotransmission polypeptides comprises a Gasdermin or a functional fragment thereof.

76. The virus of claim 74 or 75, wherein the Gasdermin is Gasdermin E or a functional fragment thereof.

77. The virus of any one of claims 1 to 76, wherein the one or more polynucleotides further comprises a polynucleotide encoding a dimerization domain.

78. The virus of any one of claims 1 to 77, wherein at least one of the thanotransmission polypeptides is comprised within a fusion protein that further comprises a dimerization domain.

79. The virus of claim 77 or 78, wherein the dimerization domain is heterologous to the thanotransmission polypeptide.

80. A pharmaceutical composition comprising the virus of any one of claims 1 to 79, and a pharmaceutically acceptable carrier.

81. A method of delivering one or more thanotransmission polynucleotides to a subject, the method comprising administering the pharmaceutical composition of claim 80 to the subject.

82. A method of promoting thanotransmission in a subject, the method comprising administering the pharmaceutical composition of claim 80 to the subject in an amount and for a time sufficient to promote thanotransmission.

83. A method of increasing immune response in a subject in need thereof, the method comprising administering the pharmaceutical composition of claim 80 to the subject in an amount and for a time sufficient to increase immune response in the subject.

84. A method of treating a cancer in a subject in need thereof, the method comprising administering the pharmaceutical composition of claim 80 to the subject in an amount and for a time sufficient to treat the cancer.

85. The method of claim 84, wherein administering the pharmaceutical composition to the subject reduces proliferation of cancer cells in the subject.

86. The method of claim 85, wherein the proliferation of the cancer cells is a hyperproliferation of the cancer cells resulting from a cancer therapy administered to the subject.

87. The method of any one of claims 84 to 86, wherein administering the pharmaceutical composition to the subject reduces metastasis of cancer cells in the subject.

88. The method of any one of claims 84 to 87, wherein administering the pharmaceutical composition to the subject reduces neovascularization of a tumor in the subject.

89. The method of any one of claims 84 to 88, wherein treating a cancer comprises any one or more of reduction in tumor burden, reduction in tumor size, inhibition of tumor growth, achievement of stable cancer in a subject with a progressive cancer prior to treatment, increased time to progression of the cancer, and increased time of survival.

90. The method of any one of claims 81 to 89, wherein the pharmaceutical composition is administered intravenously to the subject.

91. The method of any one of claims 81 to 89, wherein the pharmaceutical composition is administered intratumorally to the subject.

92. The method of any one of claims 81 to 91, wherein the subject was previously treated with an immunotherapy.

93. The method of any one of claims 84 to 92, wherein the cancer is not responsive to an immunotherapy.

94. The method of any one of claims 84 to 92, wherein the cancer is a cancer responsive to an immunotherapy.

95. The method of any one of claims 84 to 94, wherein administration of the pharmaceutical composition to the subject improves response of the cancer to an immunotherapy relative to a subject that is administered the immunotherapy but is not administered the virus.

96. The method of claim 95, wherein the immunotherapy is an immune checkpoint therapy.

97. The method of claim 96, wherein the immune checkpoint therapy is an immune checkpoint inhibitor therapy.

98. The method of any one of claims 84 to 97, wherein the cancer is selected from a carcinoma, sarcoma, lymphoma, melanoma, and leukemia.

99. The method of any one of claims 84 to 97, wherein the cancer is a solid tumor.

100. The method of any one of claims 84 to 97, wherein the cancer is selected from the group consisting of melanoma, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplasia syndrome, multiple myeloma, transitional cell carcinoma, neuroblastoma, plasma cell neoplasms, Wilm's tumor, and hepatocellular carcinoma.

101. The method of any one of claims 84 to 97, wherein the cancer is colon cancer.

102. The method of any one of claims 84 to 101, wherein the method further comprises administering an anti-neoplastic agent to the subject.

103. The method of claim 102, wherein the anti-neoplastic agent is a chemotherapeutic agent.

104. The method of claim 102, wherein the anti-neoplastic agent is a biologic agent.

105. The method of claim 104, wherein the biologic agent is an antigen binding protein.

106. The method of claim 102, wherein the anti-neoplastic agent is an immunotherapeutic.

107. The method of claim 106, wherein the immunotherapeutic is selected from the group consisting of a Toll-like receptor (TLR) agonist, a cell-based therapy, a cytokine, a cancer vaccine, and an immune checkpoint modulator of an immune checkpoint molecule.

108. The method of claim 107, wherein the TLR agonist is selected from Coley's toxin and Bacille Calmette-Guérin (BCG).

109. The method of claim 107, wherein the cell-based therapy is a chimeric antigen receptor T cell (CAR-T cell) therapy.

110. The method of claim 107, wherein the immune checkpoint molecule is selected from CD27, CD28, CD40, CD122, OX40, GITR, ICOS, 4-1BB, ADORA2A, B7-H3, B7-H4, BTLA, CTLA-4, IDO, KIR, LAG-3, PD-1, PD-L1, PD-L2, TIM-3, and VISTA.

111. The method of claim 107, wherein the immune checkpoint molecule is a stimulatory immune checkpoint molecule and the immune checkpoint modulator is an agonist of the stimulatory immune checkpoint molecule.

112. The method of claim 107, wherein the immune checkpoint molecule is an inhibitory immune checkpoint molecule and the immune checkpoint modulator is an antagonist of the inhibitory immune checkpoint molecule.

113. The method of claim 107, wherein the immune checkpoint modulator is selected from a small molecule, an inhibitory RNA, an antisense molecule, and an immune checkpoint molecule binding protein.

114. The method of claim 107, wherein the immune checkpoint molecule is PD-1 and the immune checkpoint modulator is a PD-1 inhibitor.

115. The method of claim 114, wherein the PD-1 inhibitor is selected from pembrolizumab, nivolumab, pidilizumab, SHR-1210, MEDI0680R01, BBg-A317, TSR-042, REGN2810 and PF-06801591.

116. The method of claim 107, wherein the immune checkpoint molecule is PD-L1 and the immune checkpoint modulator is a PD-L1 inhibitor.

117. The method of claim 116, wherein the PD-L1 inhibitor is selected from durvalumab, atezolizumab, avelumab, MDX-1105, AMP-224 and LY3300054.

118. The method of claim 107, wherein the immune checkpoint molecule is CTLA-4 and the immune checkpoint modulator is a CTLA-4 inhibitor.

119. The method of claim 118, wherein the CTLA-4 inhibitor is selected from ipilimumab, tremelimumab, JMW-3B3 and AGEN1884.

120. The method of claim 102, wherein the anti-neoplastic agent is a histone deacetylase inhibitor.

121. The method of claim 120, wherein the histone deacetylase inhibitor is a hydroxamic acid, a benzamide, a cyclic tetrapeptide, a depsipeptide, an electrophilic ketone, or an aliphatic compound.

122. The method of claim 121, wherein the hydroxamic acid is vorinostat (SAHA), belinostat (PXD101), LAQ824, trichostatin A, or panobin ostat (LBH589).

123. The method of claim 121, wherein the benzamide is entinostat (MS-275), 01994, or mocetinostat (MGCD0103).

124. The method of claim 121, wherein the cyclic tetrapeptide is trapoxin B.

125. The method of claim 121, wherein the aliphatic acid is phenyl butyrate or valproic acid.

126. The virus of any one of claims 1 to 79, the pharmaceutical composition of claim 80, or the method of any one of claims 81 to 125, wherein the virus is not an adenovirus or an adeno-associated virus (AAV).

127. The virus of any one of claims 1 to 79, the pharmaceutical composition of claim 80, or the method of any one of claims 81 to 125, wherein the virus is cytolytic.

128. The virus of any one of claims 1 to 79, the pharmaceutical composition of claim 80, or the method of any one of claims 81 to 125, wherein the virus preferentially infects dividing cells.

129. The virus of any one of claims 1 to 79, the pharmaceutical composition of claim 80, or the method of any one of claims 81 to 125, wherein the virus is capable of reinfecting a host that was previously infected.

130. The virus of any one of claims 1 to 79, the pharmaceutical composition of claim 80, or the method of any one of claims 81 to 125, wherein the virus does not comprise a polynucleotide encoding a synthetic multimerization domain.

131. The virus of any one of claims 1 to 79, the pharmaceutical composition of claim 80, or the method of any one of claims 81 to 125, wherein the virus is not a Vaccinia virus.

132. The virus of any one of claims 1 to 79, the pharmaceutical composition of claim 80, or the method of any one of claims 81 to 125, wherein the virus does not comprise a polynucleotide encoding TRIF.

133. The method of any one of claims 81 to 125 wherein an immuno-stimulatory cell turnover pathway is induced in the target cell.

134. The method of claim 133, wherein the immuno-stimulatory cell turnover pathway is selected from the group consisting of necroptosis, extrinsic apoptosis, ferroptosis, pyroptosis and combinations thereof.

135. The method of claim 133 or 134, wherein the target cell is deficient in the immuno-stimulatory cell turnover pathway.

136. The method of claim 135, wherein the target cell has an inactivating mutation in one or more of a gene encoding receptor-interacting serine/threonine-protein kinase 3 (RIPK1), a gene encoding receptor-interacting serine/threonine-protein kinase 3 (RIPK3), a gene encoding Z-DNA-binding protein 1 (ZBP1), a gene encoding mixed lineage kinase domain like pseudokinase (MLKL), and a gene encoding Toll/interleukin-1 receptor (TIR)-domain-containing adapter-inducing interferon-β (TRIF).

137. The method of claim 135, wherein the target cell has reduced expression or activity of one or more of RIPK1, RIPK3, ZBP1, TRIF, and MLKL.

138. The method of claim 135, wherein the target cell has copy number loss of one or more of a gene encoding RIPK1, a gene encoding RIPK3, a gene encoding ZBP1, a gene encoding TRIF, and a gene encoding MLKL.

139. The method of any one of claims 133 to 138, wherein the target cell is selected from the group consisting of a cancer cell, an immune cell, an endothelial cell and a fibroblast.

140. The method of claim 139, wherein the target cell is a cancer cell.

141. The method of claim 140, wherein the cancer is a metastatic cancer.

142. The virus of any one of claims 1 to 79, the pharmaceutical composition of claim 80, or the method of any one of claims 81 to 125 and 133 to 141, wherein the virus is an oncolytic virus.

143. The virus of any one of claims 1 to 79, the pharmaceutical composition of claim 80, or the method of any one of claims 81 to 125 and 133 to 141, wherein the virus is a DNA replicative virus.

144. The virus of any one of claims 1 to 79, the pharmaceutical composition of claim 80, or the method of any one of claims 81 to 125 and 133 to 141, wherein the virus is a DNA replicative oncolytic virus.

145. The virus of any one of claims 1 to 79, the pharmaceutical composition of claim 80, or the method of any one of claims 81 to 125 and 133 to 141, wherein the virus preferentially infects a target cell.

146. The virus of any one of claims 1 to 79, the pharmaceutical composition of claim 80, or the method of any one of claims 81 to 125 and 133 to 141, wherein the virus comprises inactivating mutations in one or more endogenous viral genes that inhibit thanotransmission by a cancer cell.

147. The virus of any one of claims 1 to 79, the pharmaceutical composition of claim 80, or the method of any one of claims 81 to 125 and 133 to 141, wherein the virus is capable of transporting a heterologous polynucleotide of at least 4 kb into a target cell.

148. The virus of any one of claims 1 to 79, the pharmaceutical composition of claim 80, or the method of any one of claims 81 to 125 and 133 to 141, wherein the virus is herpes simplex virus (HSV).

149. The virus, pharmaceutical composition or method of claim 148, wherein the HSV is HSV1.

150. The virus, pharmaceutical composition or method of claim 149, wherein the HSV1 is selected from the group consisting of Kos, F1, MacIntyre, McKrae and related strains.

151. The virus, pharmaceutical composition or method of any one of claims 148 to 150, wherein the HSV is defective in one or more genes selected from the group consisting of ICP34.5, ICP47, UL24, UL55, UL56.

152. The virus, pharmaceutical composition or method of claim 151, wherein each ICP34.5 encoding gene is replaced by a polynucleotide cassette comprising a US 11 encoding gene operably linked to an immediate early (IE) promoter.

153. The virus, pharmaceutical composition or method of any one of claims 148 to 152, wherein the HSV comprises a ΔZα mutant form of a Vaccinia virus E3L gene.

154. The virus, pharmaceutical composition or method of any one of claims 148 to 153, wherein the HSV is defective in one or more functions of ICP6.

155. The virus, pharmaceutical composition or method of claim 154, wherein the ICP6 has a mutation of the receptor-interacting protein homotypic interaction motif (RHIM) domain.

156. The virus, pharmaceutical composition or method of claim 154 or 155, wherein the ICP6 has one or more mutations at the C-terminus that inhibit caspase-8 binding.

157. The virus, pharmaceutical composition or method of any one of claims 154 to 156, wherein the HSV expresses the US11 gene as an immediate early gene.

158. The virus, pharmaceutical composition or method of any one of claims 154 to 156, wherein the ICP47 gene is deleted such that the US11 gene is under the control of an ICP47 immediate early promoter.

159. The virus of any one of claims 1 to 79, the pharmaceutical composition of claim 80, or the method of any one of claims 81 to 125 and 133 to 141, wherein the virus belongs to the Poxviridae family.

160. The virus, pharmaceutical composition or method of claim 159, wherein the virus that belongs to the Poxviridae family is selected from the group consisting of myxoma virus, Yaba-like disease virus, raccoonpox virus, orf virus and cowpox virus.

161. The virus, pharmaceutical composition or method of claim 159, wherein the virus belongs to the Chordopoxvirinae subfamily of the Poxviridae family.

162. The virus, pharmaceutical composition or method of claim 161, wherein the virus belongs to the Orthopoxvirus genus of the Chordopoxvirinae subfamily.

163. The virus, pharmaceutical composition or method of claim 162, wherein the virus belongs to the Vaccinia virus species of the Orthopoxvirus genus.

164. The virus, pharmaceutical composition or method of claim 163, wherein the Vaccinia virus is a strain selected from the group consisting of Dairenl, IHD-J, L-IPV, LC16M8, LC16MO, Lister, LIVP, Tashkent, WR 65-16, Wyeth, Ankara, Copenhagen, Tian Tan and WR.

165. The virus, pharmaceutical composition or method of claim 163 or 164, wherein the Vaccinia virus is engineered to lack thymidine kinase (TK) activity.

166. The virus, pharmaceutical composition or method of any one of claims 163 to 165, wherein the Vaccinia virus has an inactivating mutation or deletion in the J2R gene that reduces or eliminates TK activity.

167. The virus, pharmaceutical composition or method of any one of claims 163 to 166, wherein the Vaccinia virus is engineered to lack ribonucleotide reductase (RR) activity.

168. The virus, pharmaceutical composition or method of claim 167, wherein the Vaccinia virus has an inactivating mutation or deletion in a gene selected from I4L and F4L gene that reduces or eliminates RR activity.

169. The virus, pharmaceutical composition or method of any one of claims 163 to 168, wherein the Vaccinia virus is defective in the E3L gene.

170. The virus, pharmaceutical composition or method of claim 169, wherein the E3L gene has a mutation that results in induction of necroptosis in the cancer cell.

171. The virus of any one of claims 1 to 79, the pharmaceutical composition of claim 80, or the method of any one of claims 81 to 125 and 133 to 141, wherein the virus is an adenovirus.

172. The virus, pharmaceutical composition or method of claim 171, wherein the adenovirus is Ad5/F35.

173. The virus, pharmaceutical composition or method of claim 171 or 172, wherein the adnovirus comprises a deletion in the Adenovirus Early Region 1A (E1A).

174. The virus, pharmaceutical composition or method of any one of claims 171 to 173, wherein the adenovirus comprises a deletion in the Adenovirus Early Region 1B (E1B).

175. The virus, pharmaceutical composition or method of any one of claims 171 to 174 wherein the adenovirus has an Arg-Gly-Asp (RGD)-motif engineered into a fiber-H loop.
